(19) United States
(12) Patent Application Publication
(10) Pub. No.: US 2025/0228851 A1
(43) Pub. Date: Jul. 17, 2025

(54) ALLOSTERIC INHIBITOR COMPOUNDS FOR OVERCOMING CANCER RESISTANCE

(71) Applicant: Yousef NAJAJREH, Shmeisani, Amman (JO)

(72) Inventor: Yousef NAJAJREH, Shmeisani, Amman (JO)

(21) Appl. No.: 18/703,228

(22) PCT Filed: Oct. 20, 2022
(86) PCT No.: PCT/IB2022/060106

§ 371 Date: Apr. 19, 2024

Related U.S. Application Data
(60) Provisional application No. 63/257,848, filed on Oct. 20, 2021.

(51) Int. Cl. A61K 31/506; A61P 35/00; C07D 239/48; C07D 401/12; C07D 403/12

(52) U.S. Cl. CPC A61K 31/506; A61P 35/00; C07D 239/48; C07D 401/12; C07D 403/12

(57) ABSTRACT

The present invention provides allosteric inhibitor compounds for overcoming cancer resistance, or treating or preventing cancer, wherein the compounds of the present invention are administered alone or in combination with known chemotherapeutic agents. Representative compounds of the invention are compounds of structural Formula (I).

### FIELD OF THE INVENTION

The present invention pertains to the field of cancer therapeutics, and in particular to compounds that overcome cancer resistance.

### BACKGROUND

Leukemia is a complex disease that encompasses different subtypes: Acute myeloid (or myelogenous) leukemia (AML), Chronic myeloid (or myelogenous) leukemia (CML), and Acute lymphocytic (or lymphoblastic) leukemia (ALL). In leukemia as well as Myeloproliferative Neoplasms (MPN), Chronic lymphocytic leukemia (CLL) with AML and CLL as most frequent among adults. Activated JAK2 and BCR and ABL kinases are two distinct kinases such as BCR-ABL, CAN/ABL or V617PJAK2 play an important role for the pathogenesis. However, both phosphorylate a shared downstream substrate e.g., the signal transducer and activator of transcription 5 (STAT5).

Leukemia is considered challenging sickness to treat. So far, the mainstream treatment for this disease is traditional systemic combinatorial chemotherapy able to induce a high rate of complete remission, but unfortunately followed by a high rate of relapses and resulting in resistance. Resistance and life-threatening side effects of the chemotherapy prove the urgent need of novel targeted molecular therapy approaches for the treatment of blood cancer. Progress in leukemia treatment has been slow over the past 40 years, but clinical trials are exploring therapies that target specific molecular genetic changes in the disease. As leukemia continues to be better understood and disease mechanisms can be used to target the disease more precisely, new treatments have the potential to improve outcomes and, therefore, may help to relieve burdens on the healthcare system. Many of the clinical trials examine the safety of therapy in leukemia patients in three main approaches: development of new drugs, combinational approach through a combination of standard treatments, or trying new doses of standard drugs.

The major clinical challenge of current anticancer chemotherapies in general, and not only of antileukemic agents in particular, is the emergence of resistant strains that cause the relapse and as a consequence patient death. Resistance is a multifactorial and complicated process, but one well established mechanism that hindered the efficacy of selective targeted therapeutics is the selection for irresponsive mutants that render the available drugs inefficacious. The most prominent mechanisms are the up-coming of resistance mutants such as the widely reported resistant mutants in for BCR-ABL driven leukemia cells [1-3]. The mutations mostly involved are located at the kinase domain encompassing modifying the binding site of most approved tyrosine kinase inhibitors (TKIs).

Importantly, it was noticed that analogous mutations at the kinase domain can induce resistance to numerous drugs that originally act by interaction to similar binding regions. For example, the irresponsiveness of certain BCR-ABL mutants like T315I-BCR-ABL to first- and second-generation TKIs led to the development of ponatinib as a third-generation inhibitor [4-7]. However, ponatinib can cause, apart from cardiovascular problems, hepatotoxicity including fulminant hepatic failure and even deaths after a period of repeated treatment. Hence it could be concluded that, despite the approval of a range of molecular targeted TKIs, there is still the urgent need for novel molecular therapy approaches for treatment of leukemia and other blood cancers such as MPNs is still marked as undefeated sickness and thus poses a great clinical challenge to be combated [8-10].

Most of the clinically approved TKIs are defined as ATP competitors that bind at the kinase domain [1, 43-46]. Design and development of ATP competitive inhibitors suffers the shortcoming of targeting homologous sequences comprising the common binding site among more than 500 known kinases. This homology is believed to lay the molecular basis for the emergence of resistance and the side effects exerted by many TKIs. Such side effects can be as serious as cardiovascular-, hepato-, brain- or nephrotoxicity.

Studies revealed that the kinase domain is recurrently subjected to mutations that are clinically relevant rendering the drugs ineffective [47-52]. Additionally, due the complexity of cancer disease, monotherapy (administration of a single agent) is rarely effective. Thus, chemotherapy regimens follow the combinational approach for seeking drug additive and synergistic effect.

CML (chronic myeloid leukemia), a myeloproliferative neoplasm with an incidence of 1-2 cases per 100 000 adults, and accounts for approximately 15% of newly diagnosed cases of leukemia in adults. In recent years, some studies indicate improvement in survival rates of CML, the disease having been transformed from a fatal disease to a chronic disease [26]. This requires life-long therapy with ABL-directed TKIs, with all their long-term side effects.

As a result, and in the US alone the annual cost of managing the disease is projected to rise to $5.1 billion by 2025, up from $740,000 million in 2011, based on current pricing. And the cost of treating each patient for a lifetime is expected to increase 310 percent, to $604,000 from $147,000 during that same period. And for Medicare patients, the out-of-pocket cost is forecast to jump 520 percent, to $57,000 from $9,200 that is further compounded by a therapy-induced increased prevalence of CML. In addition to these economic considerations, patients are burdened by long-term side effects of indefinite TKI therapy. Discontinuation clinical trials evidenced only a small group of excellent responders, approximately 60% of them eventually have to resume treatment because of molecular relapse. This clearly proves the inability of these TKIs to eradicate the cancer stem cells [27-30]. Treatment that can achieve this goal would therefore have a dramatic beneficial impact on patient's quality of life and a huge economic impact by eliminating the need for life-long therapy.

Ph+ ALL on the other hand is a highly aggressive subset (25-30%) of ALL and can be controlled by ABL-directed TKI for an only limited period of time [31-33]. The development of BCR-ABL kinase domain mutations block approved ATP-competitor TKIs, resulting in emergence of drug-resistant subclones and relapse in the vast majority of patients. Thus, median survival of patient is a few months [34,35]. Allogenic HSCT is currently considered the only definitively curative therapy, but is associated with substantial morbidity, as well as transplant-related mortality in the range of 30% [36]. Moreover, the higher median age of patients with Ph+ ALL compared with adult ALL as a whole makes many patients ineligible for HSCT, or results in substantially poorer outcome [13]. Thus, development of drug therapy that counteracts the adverse impact of TKD mutations and more effectively targets the LIC responsible for relapse would obviate the need for HSCT with its inherent risks and enable curative therapy. Ideally, treatment could be of limited duration.

In malignant diseases activated ABL-kinases (ABL or ARG, BCR-ABL, ETV6-ABL, NUP214-ABL) regulate signalling pathways that control proliferation, survival, invasion, adhesion and migration. Apart from leukemia [14,15] activated ABL has been detected in breast-, colon-, lung- and kidney carcinoma cells [16-19]. It plays a critical role in invasion and metastasis in breast and lung cancer. MBP binders, such as GNF5, are able to revert metastasis formation in models of both breast and lung cancer. The fact that ABL-inhibition by imatinib interrupts the process of synaptic loss induced by amyloid-3 oligomers and releases the block of LTP induction suggests a role of ABL in the pathogenesis of neurodegenerative disease in particular Alzheimer's and Parkinson's diseases [20-25].

Although targeting BCR-ABL with TKIs is a proven concept for the treatment of Ph+ leukemias, resistance attributable to either mutations in BCR-ABL or non-mutational mechanisms remains the major clinical challenge. Even ponatinib, the only approved TKI able to inhibit the “gatekeeper” mutation T315I, presents frequent and sometimes life-threatening cardiovascular side effects attributed to its broad spectrum and related off target effects [37].

Allosteric inhibition of ABL: 1) increases selectivity by its binding to a less common than the ATP-binding site of a kinase; 2) allows a combination with TKIs; and 3) can overcome resistance of BCR-ABL mutants by the induction of conformational changes. In the case of Myristoyl Capping Mimetic (MCM) it has the additional effect that in Ph+ leukemia not only the driver mutation BCR-ABL is targeted, but also a direct and indispensable substrate, the JAK2-STAT5 cascade. This also extends its target profile to another disease entity the myeloproliferative neoplasms (MPN), such as polycythaemia very (PV), essential thrombocytosis or primary myelofibrosis (PMF) are in the great majority driven by JAK2 mutations: 95% of PV and approx. 50% of ET and PMF harbour V617F-JAK. 1-5% of V617F-JAK2 negative patients present JAK2 with exon 12 mutations. Targeting the aberrantly active JAK2 by ruxolitinib is the first approach of molecular therapy for MPNs. Furthermore, activated JAK2 is critical in the pathogenesis of inflammatory diseases, autoimmune disorders such as rheumatoid arthritis [38-42].

These data, together with the development of resistance in Ph+ leukemia, underline the urgent need for novel approaches beyond compounds targeting the catalytic action of the oncoproteins V617-JAK2 or BCR-ABL or its resistance mutants.

Asciminib (also referred to as ABL001) is an orally bioavailable, allosteric BCR-ABL tyrosine kinase inhibitor (TKI) with potential antineoplastic activity. ABL001 binds to the AbN portion of the BCR-ABL fusion protein at a location that is distinct from the ATP-binding domain.

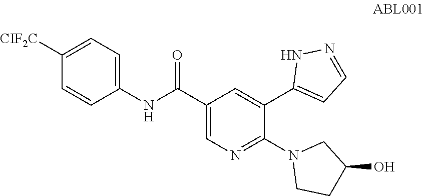

Since most clinically relevant mutations were reported to occur within the kinase domain, there is therefore a need to overcome such complication by providing innovative treatments that target allosteric regions residing outside the orthosteric ATP binding site. This would allow inhibition of the master oncogenic BCR-ABL mutants in Ph+ leukemia and other leukemias, as well as the JAK2 signalling which holds the potential of extending the spectrum of targeted diseases orchestrated by such enzyme and has its major role in the determination of resistance in Ph+ leukemia. The development of such novel therapeutics has the potential to improve outcomes and, therefore, may help to relieve burdens on the healthcare system.

This background information is provided to reveal information believed by the applicant to be of possible relevance to the present invention. No admission is necessarily intended, nor should be construed, that any of the preceding information constitutes prior art against the present invention.

### SUMMARY OF THE INVENTION

An object of the present invention is to provide allosteric inhibitor compounds for overcoming cancer resistance. In accordance with an aspect of the present invention, there is provided a compound having the structural Formula (I):

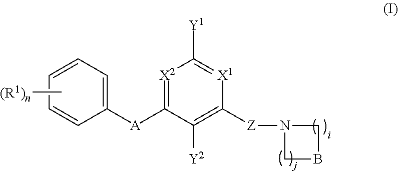

or a pharmaceutically acceptable salt thereof,
wherein:
X¹ and X² are each independently N or CH;
A is —NH—, —N(CH₃)—, —N(OH)—, —N(OCH₃)—, —NHC(O)—, —OC(O)NH—, —NHC(O)NH—, —NHS(O)₂—, —NHS(O)₂NH—, —S—, —O—, or —CH₂—, C(O)—, —S(O)—, or —S(O)₂—;
Z is absent, —NH—, —N(CH₃)—, —N(OH)—, —N(OCH₃)—, —C(O)—, —SO₂—, —NHC(O)—, —NH(CO)NH—, —NHS(O)₂—, —NHS(O)₂NH—, —CH₂—, —SO—, a benzoyl moiety, or a phenyl moiety,
Y₁ and Y₂ are each independently hydrogen, halogen, C₁₋₁₆alkyl, OH, —O—C₁₋₁₆alkyl, —S—C₁₋₁₆alkyl, —S(O)—C₁₋₁₆alkyl, —S(O₂)—C₁₋₁₆alkyl, —C(O)—C₁₋₁₆alkyl, a cycloalkyl group, a heterocyclic group, a heteroaromatic group, or an aryl group, wherein the —C₁₋₁₆alkyl, —O—C₁₋₁₆alkyl, cycloalkyl group, S—C₁₋₁₆alkyl, —S(O)—C₁₋₁₆alkyl, —S(O₂)—C₁₋₁₆alkyl, —C(O)—C₁₋₁₆alkyl, heterocyclic group, heteroaromatic group, and aryl group are optionally substituted with one or more substituents independently selected from halogen, hydroxyl, oxo, lower alkyl, lower alkoxyl, and phenyl;
B is —NR² or —CHR²;
R² is —Y—R³;
Y is absent, —C(O)—, —SO—, —SO₂—, or —(CO)NH—;
R³ is hydrogen, —C₁₋₁₆alkyl, —O—C₁₋₁₆alkyl, —S—C₁₋₁₆alkyl, —S(O)—C₁₋₁₆alkyl, —S(O)₂—C₁₋₁₆alkyl, —C(O)—C₁₋₁₆alkyl, a cycloalkyl group, a heterocyclic group, a heteroaromatic group, or an aryl group, wherein the —C₁₋₁₆alkyl, —O—C₁₋₁₆alkyl, cycloalkyl group, —S—C₁₋₁₆alkyl, —S(O)—C₁₋₁₆alkyl, —S(O)₂—C₁₋₁₆alkyl, —C(O)—C₁₋₁₆alkyl, heterocyclic group, heteroaromatic group, and aryl group are optionally substituted with one or more substituents independently selected from halogen, hydroxyl, oxo, lower alkyl, lower alkoxyl, halogenated lower alkyl, halogenated lower alkoxy, and phenyl;
i is 1, 2, 3 or 4;
j is 1, 2, 3 or 4;
R¹ is halogen, hydroxyl, —C₁₋₁₆alkyl, —O—C₁₋₁₆alkyl, S—C₁₋₁₆alkyl, —S(O)—C₁₋₁₆alkyl, —S(O)₂—C₁₋₁₆alkyl, —C(O)—C₁₋₁₆alkyl or —N(R^{b})₂, wherein R^{b} is —H or —C₁₋₆alkyl, wherein each alkyl is optionally substituted with one or more halogens selected from F, Cl and Br; and
n is 0, 1, 2 or 3.

In accordance with another aspect of the present invention, there is provided a compound having the structural Formula (II):

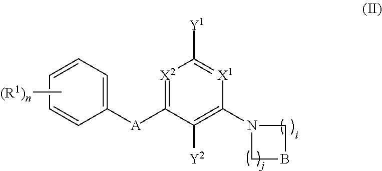

or a pharmaceutically acceptable salt thereof,
wherein:
X¹ and X² are each independently N or CH;
A is —NH—, —N(CH₃)—, —N(OH)—, —N(OCH₃)—, —NHC(O)—, —OC(O)NH—, —NHC(O)NH—, —NHS(O)₂—, —NHS(O)₂NH—, —S—, —O—, or —CH₂—, C(O)—, —S(O)—, or —S(O)₂—; Y¹ and Y² are independently hydrogen, halogen, C₁₋₁₆alkyl, —O—C₁₋₁₆alkyl, —S—C₁₋₁₆alkyl, —S(O)—C₁₋₁₆alkyl, —S(O)₂—C₁₋₁₆alkyl, —C(O)—C₁₋₁₆alkyl, a cycloalkyl group, a heterocyclic group, a heteroaromatic group, or an aryl group, wherein the —C₁₋₁₆alkyl, —O—C₁₋₁₆alkyl, cycloalkyl group, S—C₁₋₁₆alkyl, —S(O)—C₁₋₁₆alkyl, —S(O)₂—C₁₋₁₆alkyl, —C(O)—C₁₋₁₆alkyl, heterocyclic group, heteroaromatic group, and aryl group are optionally substituted with one or more substituents independently selected from halogen, hydroxyl, oxo, lower alkyl, lower alkoxyl, and phenyl;
B is —NR² or —CHR²;
R² is —Y—R³;
Y is absent, —C(O)—, —SO—, —SO₂—, or —(CO)NH—;
R³ is hydrogen, —C₁₋₁₆alkyl group, —O—C₁₋₁₆alkyl group, —S—C₁₋₁₆alkyl group, —S(O)—C₁₋₁₆alkyl group, —S(O)₂—C₁₋₁₆alkyl group, —C(O)—C₁₋₁₆alkyl group, a cycloalkyl group, a heterocyclic group, a heteroaromatic group, or an aryl group, wherein the —C₁₋₁₆alkyl group, —O—C₁₋₁₆alkyl group, cycloalkyl group, S—C₁₋₁₆alkyl group, —S(O)—C₁₋₁₆alkyl group, —S(O)₂—C₁₋₁₆alkyl group, —C(O)—C₁₋₁₆alkyl group, heterocyclic group, heteroaromatic group, and aryl group are optionally substituted with one or more substituents independently selected from halogen, hydroxyl, oxo, lower alkyl, lower alkoxyl, halogenated lower alkyl, halogenated lower alkoxy, and phenyl;
i is 1, 2, 3 or 4;
j is 1, 2, 3 or 4;
R¹ is halogen, hydroxyl, —C₁₋₁₆alkyl, —O—C₁₋₁₆alkyl, S—C₁₋₁₆alkyl group, —S(O)—C₁₋₁₆alkyl group, —S(O₂)—C₁₋₁₆alkyl group, —C(O)—C₁₋₁₆alkyl group or —N(R^{b})₂, wherein R^{b} is —H or —C₁₋₆alkyl, wherein each alkyl is optionally substituted with one or more halogens selected from F, Cl and Br; and
n is 0, 1, 2 or 3.

In accordance with another aspect of the present invention, there is provided a compound having the structural Formula (III):

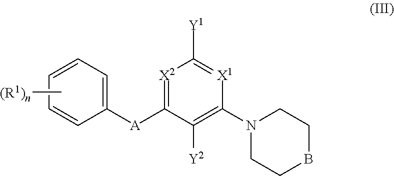

or a pharmaceutically acceptable salt thereof,
wherein:
X¹ and X² are each independently N or CH;
A is —NH—, —N(CH₃)—, —N(OH)—, —N(OCH₃)—, —NHC(O)—, —OC(O)NH—, —NHC(O)NH—, —NHS(O)₂—, —NHS(O)₂NH—, —S—, —O—, or —CH₂—, C(O)—, —S(O)—, or —S(O)₂—; Y¹ and Y² are independently hydrogen, halogen, C₁₋₁₆alkyl, —O—C₁₋₁₆alkyl, —S—C₁₋₁₆alkyl, —S(O)—C₁₋₁₆alkyl, —S(O)₂—C₁₋₁₆alkyl, —C(O)— C₁₋₁₆alkyl, a cycloalkyl group, a heterocyclic group, a heteroaromatic group, or an aryl group, wherein the —C₁₋₁₆alkyl, —O—C₁₋₁₆alkyl, cycloalkyl group, —S—C₁₋₁₆alkyl, —S(O)—C₁₋₁₆alkyl, —S(O)₂—C₁₋₁₆alkyl, —C(O)—C₁₋₁₆alkyl, heterocyclic group, heteroaromatic group, and aryl group are optionally are optionally substituted with one or more substituents independently selected from halogen, hydroxyl, oxo, lower alkyl, lower alkoxyl, and phenyl;
B is —NR² or —CHR²;
R² is —Y—R³;
Y is absent, —C(O)—, —SO—, —SO₂—, or —(CO)NH—;
R³ is hydrogen, —C₁₋₁₆alkyl, —O—C₁₋₁₆alkyl, —S—C₁₋₁₆alkyl, —S(O)—C₁₋₁₆alkyl, —S(O)₂—C₁₋₁₆alkyl, —C(O)—C₁₋₁₆alkyl, a cycloalkyl group, a heterocyclic group, a heteroaromatic group, or an aryl group, wherein the —C₁₋₁₆alkyl, —O—C₁₋₁₆alkyl, cycloalkyl group, S—C₁₋₁₆alkyl, —S(O)—C₁₋₁₆alkyl, —S(O)₂—C₁₋₁₆alkyl, —C(O)—C₁₋₁₆alkyl, heterocyclic group, heteroaromatic group, and aryl group are optionally substituted with one or more substituents independently selected from halogen, hydroxyl, oxo, lower alkyl, lower alkoxyl, halogenated lower alkyl, halogenated lower alkoxy, and phenyl;
R¹ is halogen, hydroxyl, —C₁₋₁₆alkyl, —O—C₁₋₁₆alkyl, S—C₁₋₁₆alkyl, —S(O)—C₁₋₁₆alkyl, —S(O)₂—C₁₋₁₆alkyl, —C(O)—C₁₋₁₆alkyl or —N(R^{b})₂, wherein R^{b} is —H or —C₁₋₆alkyl, wherein each alkyl is optionally substituted with one or more halogens selected from F, Cl and Br; and
n is 0, 1, 2 or 3.

In accordance with another aspect of the present invention, there is provided compound having the structural Formula (IV):

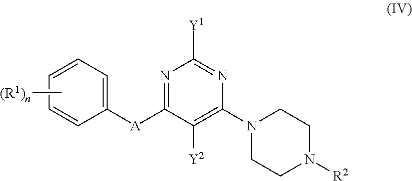

or a pharmaceutically acceptable salt thereof,
wherein:
A is —NH—, —NHC(O)—, —S—, —O—, —CH₂—, C(O)—, —S(O)—, or —S(O₂)—; Y¹ and Y² are independently hydrogen, halogen, C₁₋₁₆alkyl, —O—C₁₋₁₆alkyl, —S—C₁₋₁₆alkyl, —S(O)—C₁₋₁₆alkyl, —S(O)₂—C₁₋₆alkyl, —C(O)—C₁₋₁₆alkyl, a cycloalkyl group, a heterocyclic group, a heteroaromatic group, or an aryl group, wherein the —C₁₋₁₆alkyl, —O—C₁₋₁₆alkyl, cycloalkyl group, S—C₁₋₁₆alkyl, —S(O)—C₁₋₁₆alkyl, —S(O)₂—C₁₋₆alkyl, —C(O)—C₁₋₁₆alkyl, heterocyclic group, heteroaromatic group, and aryl group are optionally substituted with one or more substituents independently selected from halogen, hydroxyl, oxo, lower alkyl, lower alkoxyl, and phenyl;
B is —NR² or —CHR²;
R² is —Y—R³;
Y is absent, —C(O)—, —SO—, —SO₂—, or —(CO)NH—;
R³ is hydrogen, —C₁₋₁₆alkyl, —O—C₁₋₁₆alkyl, —S—C₁₋₁₆alkyl, —S(O)—C₁₋₁₆alkyl, —S(O)₂—C₁₋₁₆alkyl, —C(O)—C₁₋₁₆alkyl, a cycloalkyl group, a heterocyclic group, a heteroaromatic group, or an aryl group, wherein the —C₁₋₁₆alkyl, —O—C₁₋₁₆alkyl, cycloalkyl group, S—C₁₋₁₆alkyl, —S(O)—C₁₋₁₆ alkyl, —S(O)₂—C₁₋₆alkyl, —C(O)—C₁₋₁₆alkyl, heterocyclic group, heteroaromatic group, and aryl group are optionally substituted with one or more substituents independently selected from halogen, hydroxyl, oxo, lower alkyl, lower alkoxyl, halogenated lower alkyl, halogenated lower alkoxy, and phenyl;
R¹ is halogen, hydroxyl, —C₁₋₁₆alkyl, —O—C₁₋₁₆alkyl, S—C₁₋₁₆alkyl, —S(O)—C₁₋₁₆alkyl, —S(O)₂-C₁₋₁₆alkyl, —C(O)—C₁₋₁₆alkyl or —N(R^{b})₂, wherein R^{b} is —H or —C₁₋₆alkyl, wherein each alkyl is optionally substituted with one or more halogens selected from F, Cl and Br; and
n is 0, 1, 2 or 3.

In accordance with another aspect of the present invention, there is provided compound having the structural Formula (V):

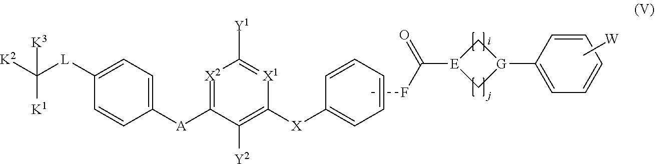

or a pharmaceutically acceptable salt thereof,
wherein:
X¹ and X² are each independently N or CH;
A is —NH—, —N(CH₃)—, —N(OH)—, —N(OCH₃)—, —NHC(O)—, —OC(O)NH—, —NHC(O)NH—, —NHS(O)₂—, —NHS(O)₂NH—, —S—, —O—, or —CH₂—, C(O)—, —S(O)—, or —S(O)₂—;
X is absent, —NH—, —N(CH₃)—, —N(OH)—, —N(OCH₃)—, —C(O)—, —SO₂—, —NHC(O)—, —NH(CO)NH—, —NHS(O)₂—, —NHS(O)₂NH—, —CH₂—, —SO—, a benzoyl moiety, or a phenyl moiety,
E is CH or N;
F is absent, —O—, —NH—, or —N(OH)—;
G is N or CH;
W is —CH₃, —CF₃, —OCF₃, OCH₂CF₃, or —CH₂CH₃;
Y¹ and Y² are each independently hydrogen, halogen, C₁₋₁₆alkyl, OH, —O—C₁₋₁₆alkyl, —S—C₁₋₁₆alkyl, —S(O)—C₁₋₁₆alkyl, —S(O₂)—C₁₋₁₆alkyl, —C(O)—C₁₋₁₆alkyl, a cycloalkyl group, a heterocyclic group, a heteroaromatic group, or an aryl group, wherein the —C₁₋₁₆alkyl, —O—C₁₋₁₆alkyl, cycloalkyl group, S—C₁₋₁₆alkyl, —S(O)—C₁₋₁₆alkyl, —S(O₂)—C₁₋₁₆alkyl, —C(O)—C₁₋₁₆alkyl, heterocyclic group, heteroaromatic group, and aryl group are optionally substituted with one or more substituents independently selected from halogen, hydroxyl, oxo, lower alkyl, lower alkoxyl, and phenyl;
R³ is hydrogen, —C₁₋₁₆alkyl, —O—C₁₋₁₆alkyl, —S—C₁₋₁₆alkyl, —S(O)—C₁₋₁₆alkyl, —S(O)₂—C₁₋₁₆ alkyl, —C(O)—C₁₋₁₆alkyl, a cycloalkyl group, a heterocyclic group, a heteroaromatic group, or an aryl group, wherein the —C₁₋₁₆alkyl, —O—C₁₋₁₆alkyl, cycloalkyl group, S—C₁₋₁₆alkyl, —S(O)—C₁₋₁₆ alkyl, —S(O)₂—C₁₋₁₆alkyl, —C(O)—C₁₋₁₆alkyl, heterocyclic group, heteroaromatic group, and aryl group are optionally substituted with one or more substituents independently selected from halogen, hydroxyl, oxo, lower alkyl, lower alkoxyl, halogenated lower alkyl, halogenated lower alkoxy, and phenyl;
i is 1, 2, 3 or 4;
j is 1, 2, 3 or 4;
L is —NH—, —NHC(O)—, —S—, —O—, —CH₂—, C(O)—, —S(O)—, or —S(O₂)—;
K¹, K² and K³ are each independently —F, —Cl, —Br, —I, —CH₃, —OH, —CF₃, —NHS(O)₂CF₃, or —NHS(O)₂CH₃.

In accordance with another aspect of the present invention, there is provided a compound having the compounds of structural Formula (VI):

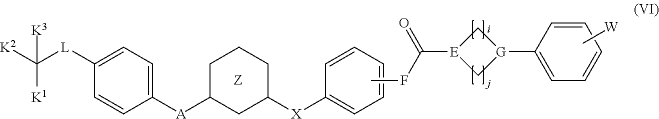

or a pharmaceutically acceptable salt thereof,
wherein:
Z is selected from

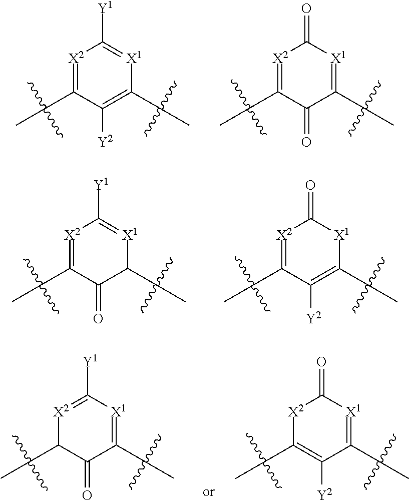

X¹ and X² are each independently N or CH;
A is —NH—, —N(CH₃)—, —N(OH)—, —N(OCH₃)—, —NHC(O)—, —OC(O)NH—, —NHC(O)NH—, —NHS(O)₂—, —NHS(O)₂NH—, —S—, —O—, or —CH₂—, C(O)—, —S(O)—, or —S(O)₂—;
X is absent, —NH—, —N(CH₃)—, —N(OH)—, —N(OCH₃)—, —C(O)—, —SO₂—, —NHC(O)—, —NH(CO)NH—, —NHS(O)₂—, —NHS(O)₂NH—, —CH₂—, —SO—, a benzoyl moiety, or a phenyl moiety,
E is CH or N;
F is absent, —O—, —NH—, or —N(OH)—;
G is N or CH;
W is —CH₃, —CF₃, —OCF₃, OCH₂CF₃, or —CH₂CH₃;
Y₁ and Y₂ are each independently hydrogen, halogen, C₁₋₁₆alkyl, OH, —O—C₁₋₁₆alkyl, —S—C₁₋₁₆alkyl, —S(O)—C₁₋₁₆alkyl, —S(O₂)—C₁₋₁₆alkyl, —C(O)—C₁₋₁₆alkyl, a cycloalkyl group, a heterocyclic group, a heteroaromatic group, or an aryl group, wherein the —C₁₋₁₆alkyl, —O—C₁₋₆alkyl, cycloalkyl group, S—C₁₋₁₆alkyl, —S(O)—C₁₋₁₆alkyl, —S(O₂)—C₁₋₁₆alkyl, —C(O)—C₁₋₁₆alkyl, heterocyclic group, heteroaromatic group, and aryl group are optionally substituted with one or more substituents independently selected from halogen, hydroxyl, oxo, lower alkyl, lower alkoxyl, and phenyl;
R³ is hydrogen, —C₁₋₁₆alkyl, —O—C₁₋₁₆alkyl, —S—C₁₋₁₆alkyl, —S(O)—C₁₋₁₆alkyl, —S(O)₂—C₁₋₁₆alkyl, —C(O)—C₁₋₁₆alkyl, a cycloalkyl group, a heterocyclic group, a heteroaromatic group, or an aryl group, wherein the —C₁₋₁₆alkyl, —O—C₁₋₁₆alkyl, cycloalkyl group, S—C₁₋₁₆alkyl, —S(O)—C₁₋₁₆alkyl, —S(O)₂—C₁₋₁₆alkyl, —C(O)—C₁₋₁₆alkyl, heterocyclic group, heteroaromatic group, and aryl group are optionally substituted with one or more substituents independently selected from halogen, hydroxyl, oxo, lower alkyl, lower alkoxyl, halogenated lower alkyl, halogenated lower alkoxy, and phenyl;
i is 1, 2, 3 or 4;
j is 1, 2, 3 or 4;
L is —NH—, —NHC(O)—, —S—, —O—, —CH₂—, C(O)—, —S(O)—, or —S(O₂)—, or —NHS(O)₂—;
K₁, K₂ and K₃ are each independently —F, —C, —Br, —I, —CH₃, —OH, CF₃, —NHS(O)₂CF₃, or —NHS(O)₂CH₃.

In accordance with another aspect of the present invention, there is provided a pharmaceutical composition comprising a compound in accordance with the present invention, and a pharmaceutically acceptable carrier or diluent.

In accordance with another aspect of the present invention, there is provided a method of treating or preventing cancer in a subject in need thereof, comprising the step of administering a therapeutically effective amount of a compound in accordance with the present invention to the subject.

In accordance with another aspect of the present invention, there is provided use of a therapeutically effective amount of a compound in accordance with the present invention for the treatment or prevention of cancer in a subject in need thereof.

In accordance with another aspect of the present invention, there is provided use of a compound in accordance with the present invention in the manufacture of a medicament for the treatment or prevention of cancer.

In accordance with another aspect of the present invention, there is provided a method of overcoming cancer resistance in a subject in need thereof, comprising the step of administering a therapeutically effective amount of a compound in accordance with the present invention to the subject

In accordance with another aspect of the present invention, there is provided use of a therapeutically effective amount of a compound in accordance with the present invention for overcoming cancer resistance in a subject in need thereof.

In accordance with another aspect of the present invention, there is provided use of a compound in accordance with the present invention in the manufacture of a medicament for overcoming cancer resistance.

In accordance with another aspect of the present invention, there is provided a method of treating or preventing cancer in a subject in need thereof, comprising the step of administering a therapeutically effective amount of a compound in accordance with the present invention to the subject, wherein the compound is administered in combination with a known chemotherapeutic agent.

In accordance with another aspect of the present invention, there is provided a method of overcoming cancer resistance in a subject in need thereof, comprising the step of administering a therapeutically effective amount of a compound in accordance with the present invention to the subject, wherein the compound is administered in combination with a known chemotherapeutic agent.

In accordance with another aspect of the present invention, there is provided use of a therapeutically effective amount of a compound in accordance with the present invention, in combination with a known chemotherapeutic agent, for the treatment or prevention of cancer in a subject in need thereof.

In accordance with another aspect of the present invention, there is provided use of a therapeutically effective amount of a compound in accordance with the present invention, in combination with a known chemotherapeutic agent, for overcoming cancer resistance in a subject in need thereof.

### BRIEF DESCRIPTION OF THE FIGURES

FIGS. 1A and 1B are graphs depicting the effects of Compound 16 on Ba/F3 cells expressing either p185-BCR-ABL or T315I-p185-BCR-ABL.

FIG. 2 is a graph depicting the effect of Compound 16 on leukemia cell lines (Jurkat, Sup-B15 and BV173).

FIG. 3 is a graph depicting the effect of Compound 16 on patient-derived long-term cultures (HP, PH, BV173 and KÖ).

FIGS. 4A and 4B are graphs depicting the effect of Compound 16 on WT-Ba/F3 compared to p185-BCR-ABL-Ba/F3.

FIGS. 5A-C are graphs depicting the time-dependent inhibition of Ph+ Jurkat cells (taken as control, FIG. 5A) and Ph+PD-LTCs Sup-B15 (FIG. 5B) and BV173 (FIG. 5C) by Compound 16.

FIG. 6 is a graph depicting the sensitivity of Sup-B15 (p185-BCR-ABL) compared to BV173 cells (p210-BCR-ABL) towards increasing concentration of Compound 16.

FIGS. 7A-C are graphs depicting the effects of Compound 16 on Ba/F3 cells expressing WT-p185-BCR-ABL.

FIG. 8 is a graph depicting the inhibition of p185-BCR-ABL Ba/F3 by Compound 16.

FIG. 9 is a graph depicting the inhibition of T315I-p185-BCR-ABL-Ba/F3 cells by Compound 16.

FIG. 10 is a graph depicting the comparison of cell viability (CV) calculated as the ratio between the number of living cells following exposure to applied concentration and the number of control cells (exposed to empty vehicles) at selected time points.

FIG. 11 is a graph depicting the antiproliferative effect of Compound 16 on PINCO transfected Ba/F3 cells.

FIGS. 12A and 12B are graphs depicting tumor reduction in animal models.

FIG. 13 is a graph depicting the effect of Compound 30 on PINCO, p185 (WT-p185-BCR-ABL, Ph+ CML), T315I-p185 (T315I-p185-BCR-ABL, Ph+ CML), Jurkat, WT-Sup-B15, RT-Sup-B15, BV, HEL, HP, PH, and KÖ.

FIG. 14 is a graph depicting the comparison of inhibitory action of Compound 30 against Ph+T315I-p185-BCR-ABL CML cells compared to Ba/F3(PINCO).

FIG. 15 is a graph depicting the concentration-response of Compound 30 against PD-LTCs HP, PH, BV and KÖ.

FIG. 16 is a graph depicting the concentration-response of Compound 30 against Jurkat, WT-Sup-B15 and RT-Sup-B15.

FIG. 17 is a graph depicting the concentration-response as measured by the cell viability (CV) following exposure to increasing dose of Compound 30.

FIG. 18 is a graph depicting the antiproliferative effect of Compound 30 on the following models: a) patient-derived long-term cell culture system (PD-LTCs): HP, BV and PH and b) on transduced Ba/F3 comprising empty vector (PINCO), p185-BCR-ABL-Ba/F3, T315I-p185-BCR-ABL-Ba/F3 and c) cell lines comprising RT-Sup-B15, BV173, Ph− V617FJAK2 HEL and Jurkat.

FIG. 19 is a graph depicting the antiproliferative effect of Compound 31 on the following models: a) patient-derived long-term cell culture system (PD-LTCs): HP, BV, PH and KÖ, and b) transduced Ba/F3 comprising empty vector (PINCO), p185-BCR-ABL-Ba/F3, T315I-p185-BCR-ABL-Ba/F3 and c) cell lines comprising RT-Sup-B15, BV173, Ph− V617FJAK2 HEL and Jurkat.

FIG. 20 is a graph depicting the antiproliferative effect of Compound 32 on the following models: a) patient-derived long-term cell culture system (PD-LTCs): HP, BV, PH and KÖ, and b) transduced Ba/F3 comprising empty vector (PINCO), p185-BCR-ABL-Ba/F3, T315I-p185-BCR-ABL-Ba/F3 and c) cell lines comprising RT-Sup-B15, BV173, Ph− V617FJAK2 HEL and Jurkat.

FIG. 21 is a graph depicting the antiproliferative effect of Compound 33 on the following models: a) patient-derived long-term cell culture system (PD-LTCs): HP, BV, PH and KÖ, and b) transduced Ba/F3 comprising empty vector (PINCO), p185-BCR-ABL-Ba/F3, T315I-p185-BCR-ABL-Ba/F3 and c) cell lines comprising RT-Sup-B15, BV173, Ph− V617FJAK2 HEL and Jurkat.

FIG. 22 is a graph depicting the effect of Compound 35 on the following models: a) patient-derived long-term cell culture system (PD-LTCs): HP, BV and PH and b) on transduced Ba/F3 comprising empty vector (PINCO), p185-BCR-ABL-Ba/F3, T315I-p185-BCR-ABL-Ba/F3 and c) cell lines comprising RT-Sup-B15, BV173, Ph− V617FJAK2 HEL and Jurkat.

FIG. 23 is a graph depicting the effect of Compound 36 on patient-derived long-term cell culture system (PD-LTCs): HP, BV and PH and on cell lines Ba/F3(PINCO), p185-BCR-ABL-Ba/F3, T315I-p185-BCR-ABL-Ba/F3 and Ph− HEL cells.

FIG. 24 is a graph depicting the effect of Compound 37 on patient-derived long-term cell culture system (PD-LTCs): HP, BV and PH and on cell lines Ba/F3(PINCO), p185-BCR-ABL-Ba/F3(PINCO), T315I-p185-BCR-ABL-Ba/F3, WT-Sup-B15, RT-Sup-B15, KÖ, and Jurkat.

FIG. 25 is a graph depicting the effect of Compound 38 on patient-derived long-term cell culture system (PD-LTCs): HP, BV and PH and on cell lines Ba/F3(PINCO), p185-BCR-ABL-Ba/F3(PINCO), T315I-p185-BCR-ABL-Ba/F3(PINCO) WT-Sup-B15, and HEL.

FIG. 26 is a graph depicting the effect of Compound 39 on patient-derived long-term cell culture system (PD-LTCs): BV HP, and PH and Ph− cell line HEL.

FIG. 27 is a graph depicting the effect of Compound 40 on patient-derived long-term cell culture system (PD-LTCs): BV, HP and PH and Ph− cell line HEL.

FIG. 28 is a graph depicting the effect of Compound 41 on patient-derived long-term cell culture system (PD-LTCs): HEL, BV, HP and PH.

FIGS. 29A-C are graphs depicting the structure activity relationship of Compounds 31, 32 and 33 against (PD-LTCs): HP and BV cultures.

FIG. 30 is a graph depicting the structure activity relationship of Compounds 31, 32 and 33 against PH cells.

FIGS. 31A-C are graphs depicting the concentration-response of Compounds 31, 32 and 33 on BV cells.

FIG. 32 is a graph depicting the concentration-response of Compounds 31, 32 and 33 on BV cells.

FIG. 33 is a graph depicting the antiproliferative effect of Compounds 31, 32 and 33 on PH and BV cells.

FIG. 34 is a graph depicting the concentration-response of Compound 34 against Ba/F3(PINCO) p185-BCR-ABL-Ba/F3, T315I-p185-BCR-ABL-Ba/F, WT-Sup-B15, RT-Sup-B15, HEL and patient-derived long-term cell culture system (PD-LTCs): BV, HP, KÖ and PH.

FIG. 35 is a graph depicting the antiproliferative effect of Compound 34.

FIG. 36 is a graph depicting the aantiproliferative effect of Compound 34 on leukemia cell lines: Ba/F3(PINCO), p185-Ba/F3, T315I-p185-Ba/F3.

FIG. 37 is a graph depicting the antiproliferative effect of Compound 34.

FIG. 38 is a graph depicting the concentration-response of Compound 34 on PD-LTCs (HP, BV, KÖ and PH).

FIG. 39 is a graph depicting the concentration-response of Compound 34 against Ph− cells: Jurkat, HEL and HP.

FIG. 40 is a Western blot analysis of HEL cells exposed to increasing concentration of Compound 34.

FIGS. 41A-B are graphs depicting the concentration-response of Compound 34 against (A) Ph+p185-BCR-ABL-Ba/F3 and (B) JAK2-HEL cells in comparison to Abl001 and ruxolitinib.

FIG. 41C is a Western blot analysis of the effect of Compound 34.

FIGS. 42A-E are graphs depicting the concentration-response of Compound 34 against resistant mutants of Ph+ BCR-ABL—Ba/F3 cells.

FIGS. 43A-D are graphs depicting the concentration-response of Compound 34 (below) compared to the Abl001 (above) against KÖ and BV cells.

FIGS. 44A-D are graphs depicting the dose-response curves for single agent treatment of imatinib, nilotinib, ABL001 (asciminib), and Compound 34.

FIG. 45 depicts dose-response matrices for combination of imatinib and ABL001 (Asciminib) against three PD-LTCs (Ph− HP, Ph+PH, or Ph+T315I-BCR-ABL KÖ).

FIG. 46 depicts the HSA synergy scores of the combination of imatinib and ABL001 against three PD-LTCs (Ph− HP, Ph+PH, or Ph+T315I-BCR-ABL KÖ).

FIG. 47 depicts dose-response matrices for combination of imatinib and Compound 34 against three PD-LTCs (Ph− HP, Ph+PH, or Ph+T315I-BCR-ABL KÖ).

FIG. 48 depicts the HSA synergy scores of the combination of imatinib and Compound 34 against three PD-LTCs (Ph− HP, Ph+PH, or Ph+T315I-BCR-ABL KÖ).

FIG. 49 depicts dose-response matrices for combination of nilotinib and ABL001 (Asciminib) against three PD-LTCs (Ph− HP, Ph+PH, or Ph+T315I-BCR-ABL KÖ).

FIG. 50 depicts the HSA synergy scores of the combination of nilotinib and ABL001 against three PD-LTCs (Ph− HP, Ph+PH, or Ph+T315I-BCR-ABL KÖ).

FIG. 51 depicts dose-response matrices for combination of nilotinib and Compound 34 against three PD-LTCs (Ph− HP, Ph+PH, or Ph+T315I-BCR-ABL KÖ).

FIG. 52 depicts the HSA synergy scores of the combination of nilotinib and Compound 34 against three PD-LTCs (Ph− HP, Ph+PH, or Ph+T315I-BCR-ABL KÖ).

### DETAILED DESCRIPTION OF THE INVENTION

The present invention relates in part to the discovery of novel compounds that are useful for the treatment or prevention of cancer. As demonstrated herein, the compounds of the present invention have been shown to be effective chemotherapeutic agents for the treatment of cancer. The compounds of the present invention have also demonstrated a synergistic effect when administered in combination with known chemotherapeutic agents.

The developmental approach taken for the present invention involves the combination of two therapeutic druggable oncogenic targets that can be modulated using a single agent drug. Firstly, allosteric inhibition of BCR-ABL circumvents the resistance mutations in the TKD of BCR-ABL by binding to a distinct region that is remote from the kinase domain and mimics the autoinhibitory conformation of ABL. Secondly, concurrent inhibition of JAK2 signaling is anticipated to augment the anti-leukemic activity of BCR-ABL inhibition. This is based on data demonstrating involvement of JAK/STAT signaling in leukemogenesis. A notable link between BCR-ABL and aberrant JAK signaling pathways was identified in BCR-ABL positive CML and Ph+ ALL demonstrating that JAK2 provides survival signals to LIC independent of BCR-ABL. Thus, combined inhibition of both pathways may lead to a curative approach to these types of leukemia, but conceptually also lends itself to combinations with classical TKI, with expected synergy. The expected specificity of allosteric inhibitors compared to ATP-competitive agents is a further advantage with respect to future combination therapy. Overall, the therapeutic concept demonstrated in the present invention has potential for substantial impact on treatment of a group of high profile leukemias by shortening treatment times, reducing chronic toxicities, increasing cure rates and reducing the need for allogeneic HSCT and as a consequence of these, reducing treatment costs.

Accordingly, and without intending to be limited by theory, it is believed that the beneficial effects of the compounds of the present invention may be due to dual allosteric inhibition of JAK2 and aberrantly activated ABL kinases such as BCR-ABL such that clinically relevant mutated BCR-ABL will induce intensified inhibitory effect on resistant blood cancers while exerting their action with milder toxicities.

As leukemia has become better understood, new, more precisely targeted treatments have the potential to improve outcomes. The present invention therefore provides novel compounds effective for the treatment of resistant leukemia and which have reduced toxicities.

In one embodiment, the present invention provides novel compounds of structural Formula (I):

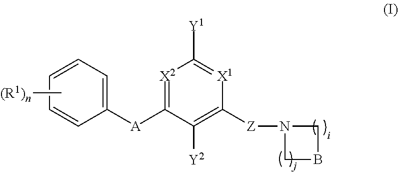

or a pharmaceutically acceptable salt thereof,
wherein:
X¹ and X² are each independently N or CH;
A is —NH—, —N(CH₃)—, —N(OH)—, —N(OCH₃)—, —NHC(O)—, —OC(O)NH—, —NHC(O)NH—, —NHS(O)₂—, —NHS(O)₂NH—, —S—, —O—, or —CH₂—, C(O)—, —S(O)—, or —S(O)₂—;
Z is absent, —NH—, —N(CH₃)—, —N(OH)—, —N(OCH₃)—, —C(O)—, —SO₂—, —NHC(O)—, —NH(CO)NH—, —NHS(O)₂—, —NHS(O)₂NH—, —CH₂—, —SO—, a benzoyl moiety, or a phenyl moiety,
Y₁ and Y₂ are each independently hydrogen, halogen, C₁₋₁₆alkyl, OH, —O—C₁₋₁₆alkyl, —S—C₁₋₁₆alkyl, —S(O)—C₁₋₁₆alkyl, —S(O₂)—C₁₋₁₆alkyl, —C(O)—C₁₋₁₆alkyl, a cycloalkyl group, a heterocyclic group, a heteroaromatic group, or an aryl group, wherein the —C₁₋₁₆alkyl, —O—C₁₋₁₆alkyl, cycloalkyl group, S—C₁₋₁₆alkyl, —S(O)—C₁₋₁₆alkyl, —S(O₂)—C₁₋₁₆alkyl, —C(O)—C₁₋₁₆alkyl, heterocyclic group, heteroaromatic group, and aryl group are optionally substituted with one or more substituents independently selected from halogen, hydroxyl, oxo, lower alkyl, lower alkoxyl, and phenyl;
B is —NR² or —CHR²;
R² is —Y—R³;
Y is absent, —C(O)—, —SO—, —SO₂—, or —(CO)NH—;
R³ is hydrogen, —C₁₋₁₆alkyl, —O—C₁₋₁₆alkyl, —S—C₁₋₁₆alkyl, —S(O)—C₁₋₁₆alkyl, —S(O)₂—C₁₋₁₆alkyl, —C(O)—C₁₋₁₆alkyl, a cycloalkyl group, a heterocyclic group, a heteroaromatic group, or an aryl group, wherein the —C₁₋₁₆alkyl, —O—C₁₋₁₆alkyl, cycloalkyl group, S—C₁₋₁₆alkyl, —S(O)—C₁₋₁₆alkyl, —S(O)₂—C₁₋₁₆alkyl, —C(O)—C₁₋₁₆alkyl, heterocyclic group, heteroaromatic group, and aryl group are optionally substituted with one or more substituents independently selected from halogen, hydroxyl, oxo, lower alkyl, lower alkoxyl, halogenated lower alkyl, halogenated lower alkoxy, and phenyl;
i is 1, 2, 3 or 4;
j is 1, 2, 3 or 4;
R¹ is halogen, hydroxyl, —C₁₋₁₆alkyl, —O—C₁₋₁₆alkyl, S—C₁₋₁₆alkyl, —S(O)—C₁₋₁₆alkyl, —S(O)₂—C₁₋₁₆alkyl, —C(O)—C₁₋₁₆alkyl or —N(R^{b})₂, wherein R^{b} is —H or —C₁₋₆alkyl, wherein each alkyl is optionally substituted with one or more halogens selected from F, Cl and Br; and
n is 0, 1, 2 or 3.

In one embodiment, the present invention provides novel compounds of structural Formula (II):

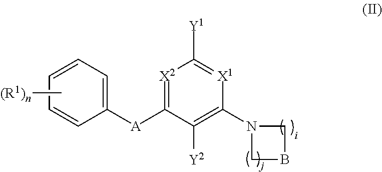

or a pharmaceutically acceptable salt thereof,
wherein:
X¹ and X² are each independently N or CH;
A is —NH—, —N(CH₃)—, —N(OH)—, —N(OCH₃)—, —NHC(O)—, —OC(O)NH—, —NHC(O)NH—, —NHS(O)₂—, —NHS(O)₂NH—, —S—, —O—, or —CH₂—, C(O)—, —S(O)—, or —S(O)₂—;
Y¹ and Y² are independently hydrogen, halogen, C₁₋₁₆alkyl, —O—C₁₋₁₆alkyl, —S—C₁₋₁₆alkyl, —S(O)—C₁₋₁₆alkyl, —S(O)₂—C₁₋₆alkyl, —C(O)—C₁₋₁₆alkyl, a cycloalkyl group, a heterocyclic group, a heteroaromatic group, or an aryl group, wherein the —C₁₋₁₆alkyl, —O—C₁₋₁₆alkyl, cycloalkyl group, S—C₁₋₁₆alkyl, —S(O)—C₁₋₁₆alkyl, —S(O)₂—C₁₋₁₆alkyl, —C(O)—C₁₋₁₆alkyl, heterocyclic group, heteroaromatic group, and aryl group are optionally substituted with one or more substituents independently selected from halogen, hydroxyl, oxo, lower alkyl, lower alkoxyl, and phenyl;
B is —NR² or —CHR²;
R² is —Y—R³;
Y is absent, —C(O)—, —SO—, —SO₂—, or —(CO)NH—;
R³ is hydrogen, —C₁₋₁₆alkyl group, —O—C₁₋₁₆alkyl group, —S—C₁₋₁₆alkyl group, —S(O)—C₁₋₁₆alkyl group, —S(O)₂—C₁₋₁₆alkyl group, —C(O)—C₁₋₁₆alkyl group, a cycloalkyl group, a heterocyclic group, a heteroaromatic group, or an aryl group, wherein the —C₁₋₁₆alkyl group, —O—C₁₋₁₆alkyl group, cycloalkyl group, S—C₁₋₁₆alkyl group, —S(O)—C₁₋₁₆alkyl group, —S(O)₂—C₁₋₁₆alkyl group, —C(O)—C₁₋₁₆alkyl group, heterocyclic group, heteroaromatic group, and aryl group are optionally substituted with one or more substituents independently selected from halogen, hydroxyl, oxo, lower alkyl, lower alkoxyl, halogenated lower alkyl, halogenated lower alkoxy, and phenyl;
i is 1, 2, 3 or 4;
j is 1, 2, 3 or 4;
R¹ is halogen, hydroxyl, —C₁₋₁₆alkyl, —O—C₁₋₁₆alkyl, S—C₁₋₁₆alkyl group, —S(O)—C₁₋₁₆alkyl group, —S(O₂)—C₁₋₁₆alkyl group, —C(O)—C₁₋₁₆alkyl group or —N(R^{b})₂, wherein R^{b} is —H or —C₁₋₆alkyl, wherein each alkyl is optionally substituted with one or more halogens selected from F, Cl and Br; and
n is 0, 1, 2 or 3.

In one embodiment, the present invention provides novel compounds of structural Formula (III):

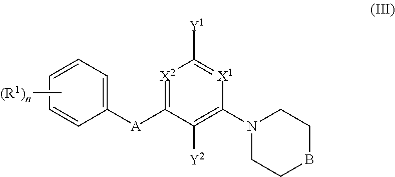

or a pharmaceutically acceptable salt thereof,
wherein:
X¹ and X² are each independently N or CH;
A is —NH—, —N(CH₃)—, —N(OH)—, —N(OCH₃)—, —NHC(O)—, —OC(O)NH—, —NHC(O)NH—, —NHS(O)₂—, —NHS(O)₂NH—, —S—, —O—, or —CH₂—, C(O)—, —S(O)—, or —S(O)₂—;
Y¹ and Y² are independently hydrogen, halogen, C₁₋₁₆alkyl, —O—C₁₋₁₆alkyl, —S—C₁₋₁₆alkyl, —S(O)—C₁₋₁₆alkyl, —S(O)₂—C₁₋₆alkyl, —C(O)—C₁₋₁₆alkyl, a cycloalkyl group, a heterocyclic group, a heteroaromatic group, or an aryl group, wherein the —C₁₋₁₆alkyl, —O—C₁₋₁₆alkyl, cycloalkyl group, S—C₁₋₁₆alkyl, —S(O)—C₁₋₁₆alkyl, —S(O)₂—C₁₋₆alkyl, —C(O)—C₁₋₁₆alkyl, heterocyclic group, heteroaromatic group, and aryl group are optionally are optionally substituted with one or more substituents independently selected from halogen, hydroxyl, oxo, lower alkyl, lower alkoxyl, and phenyl;
B is —NR² or —CHR²;
R² is —Y—R³;
Y is absent, —C(O)—, —SO—, —SO₂—, or —(CO)NH—;
R³ is hydrogen, —C₁₋₁₆alkyl, —O—C₁₋₁₆alkyl, —S—C₁₋₁₆alkyl, —S(O)—C₁₋₁₆alkyl, —S(O)₂—C₁₋₁₆alkyl, —C(O)—C₁₋₁₆alkyl, a cycloalkyl group, a heterocyclic group, a heteroaromatic group, or an aryl group, wherein the —C₁₋₁₆alkyl, —O—C₁₋₁₆alkyl, cycloalkyl group, S—C₁₋₁₆alkyl, —S(O)—C₁₋₁₆alkyl, —S(O)₂—C₁₋₆alkyl, —C(O)—C₁₋₁₆alkyl, heterocyclic group, heteroaromatic group, and aryl group are optionally substituted with one or more substituents independently selected from halogen, hydroxyl, oxo, lower alkyl, lower alkoxyl, halogenated lower alkyl, halogenated lower alkoxy, and phenyl;
R¹ is halogen, hydroxyl, —C₁₋₁₆alkyl, —O—C₁₋₁₆alkyl, S—C₁₋₁₆alkyl, —S(O)—C₁₋₁₆alkyl, —S(O)₂—C₁₋₁₆alkyl, —C(O)—C₁₋₁₆alkyl or —N(R^{b})₂, wherein R^{b} is —H or —C₁₋₆alkyl, wherein each alkyl is optionally substituted with one or more halogens selected from F, Cl and Br; and
n is 0, 1, 2 or 3.

In one embodiment, the present invention provides novel compounds of structural Formula (IV):

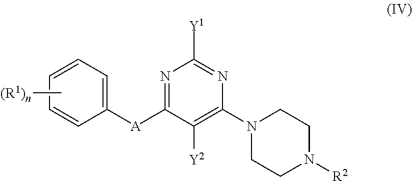

or a pharmaceutically acceptable salt thereof,
wherein:
A is —NH—, —NHC(O)—, —S—, —O—, —CH₂—, C(O)—, —S(O)—, or —S(O₂)—;
Y¹ and Y² are independently hydrogen, halogen, C₁₋₁₆alkyl, —O—C₁₋₁₆alkyl, —S—C₁₋₆alkyl, —S(O)—C₁₋₁₆alkyl, —S(O₂)—C₁₋₆alkyl, —C(O)—C₁₋₁₆alkyl, a cycloalkyl group, a heterocyclic group, a heteroaromatic group, or an aryl group, wherein the —C₁₋₆alkyl, —O—C₁₋₁₆alkyl, cycloalkyl group, S—C₁₋₁₆alkyl, —S(O)—C₁₋₁₆alkyl, —S(O)₂—C₁₋₆alkyl, —C(O)—C₁₋₁₆alkyl, heterocyclic group, heteroaromatic group, and aryl group are optionally substituted with one or more substituents independently selected from halogen, hydroxyl, oxo, lower alkyl, lower alkoxyl, and phenyl;
B is —NR² or —CHR²;
R² is —Y—R³;
Y is absent, —C(O)—, —SO—, —SO₂—, or —(CO)NH—;
R³ is hydrogen, —C₁₋₁₆alkyl, —O—C₁₋₁₆alkyl, —S—C₁₋₁₆alkyl, —S(O)—C₁₋₁₆alkyl, —S(O₂)—C₁₋₁₆alkyl, —C(O)—C₁₋₁₆alkyl, a cycloalkyl group, a heterocyclic group, a heteroaromatic group, or an aryl group, wherein the —C₁₋₆alkyl, —O—C₁₋₁₆alkyl, cycloalkyl group, S—C₁₋₁₆alkyl, —S(O)—C₁₋₁₆alkyl, —S(O₂)—C₁₋₆alkyl, —C(O)—C₁₋₁₆alkyl, heterocyclic group, heteroaromatic group, and aryl group are optionally substituted with one or more substituents independently selected from halogen, hydroxyl, oxo, lower alkyl, lower alkoxyl, halogenated lower alkyl, halogenated lower alkoxy, and phenyl;
R¹ is halogen, hydroxyl, —C₁₋₁₆alkyl, —O—C₁₋₁₆alkyl, S—C₁₋₁₆alkyl, —S(O)—C₁₋₁₆alkyl, —S(O₂)—C₁₋₁₆alkyl, —C(O)—C₁₋₁₆alkyl or —N(R^{b})₂, wherein R^{b} is —H or —C₁₋₆alkyl, wherein each alkyl is optionally substituted with one or more halogens selected from F, Cl and Br; and
n is 0, 1, 2 or 3.

In one embodiment, the present invention provides novel compounds of structural Formula (V):

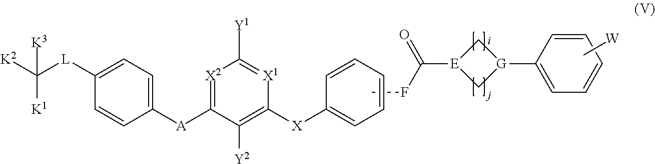

or a pharmaceutically acceptable salt thereof,
wherein:
X¹ and X² are each independently N or CH;
A is —NH—, —N(CH₃)—, —N(OH)—, —N(OCH₃)—, —NHC(O)—, —OC(O)NH—, —NHC(O)NH—, —NHS(O)₂—, —NHS(O)₂NH—, —S—, —O—, or —CH₂—, C(O)—, —S(O)—, or —S(O)₂—;
X is absent, —NH—, —N(CH₃)—, —N(OH)—, —N(OCH₃)—, —C(O)—, —SO₂—, —NHC(O)—, —NH(CO)NH—, —NHS(O)₂—, —NHS(O)₂NH—, —CH₂—, —SO—, a benzoyl moiety, or a phenyl moiety,
E is CH or N;
F is absent, —O—, —NH—, or —N(OH)—;
G is N or CH;
W is —CH₃, —CF₃, —OCF₃, OCH₂CF₃, or —CH₂CH₃;
Y¹ and Y² are each independently hydrogen, halogen, C₁₋₁₆alkyl, OH, —O—C₁₋₁₆alkyl, —S—C₁₋₁₆alkyl, —S(O)—C₁₋₁₆alkyl, —S(O₂)—C₁₋₁₆alkyl, —C(O)—C₁₋₁₆alkyl, a cycloalkyl group, a heterocyclic group, a heteroaromatic group, or an aryl group, wherein the —C₁₋₁₆alkyl, —O—C₁₋₁₆alkyl, cycloalkyl group, S—C₁₋₁₆alkyl, —S(O)—C₁₋₁₆alkyl, —S(O₂)—C₁₋₁₆alkyl, —C(O)—C₁₋₁₆alkyl, heterocyclic group, heteroaromatic group, and aryl group are optionally substituted with one or more substituents independently selected from halogen, hydroxyl, oxo, lower alkyl, lower alkoxyl, and phenyl;
R³ is hydrogen, —C₁₋₁₆alkyl, —O—C₁₋₁₆alkyl, —S—C₁₋₁₆alkyl, —S(O)—C₁₋₁₆alkyl, —S(O)₂—C₁₋₁₆alkyl, —C(O)—C₁₋₁₆alkyl, a cycloalkyl group, a heterocyclic group, a heteroaromatic group, or an aryl group, wherein the —C₁₋₁₆alkyl, —O—C₁₋₁₆alkyl, cycloalkyl group, S—C₁₋₁₆alkyl, —S(O)—C₁₋₁₆alkyl, —S(O)₂—C₁₋₁₆alkyl, —C(O)—C₁₋₁₆alkyl, heterocyclic group, heteroaromatic group, and aryl group are optionally substituted with one or more substituents independently selected from halogen, hydroxyl, oxo, lower alkyl, lower alkoxyl, halogenated lower alkyl, halogenated lower alkoxy, and phenyl;
i is 1, 2, 3 or 4;
j is 1, 2, 3 or 4;
L is —NH—, —NHC(O)—, —S—, —O—, —CH₂—, C(O)—, —S(O)—, or —S(O₂)—, or —NHS(O)₂;
K¹, K² and K³ are each independently —F, —Cl, —Br, —I, —CH₃, —OH, —CF₃, —NHS(O)₂CF₃, or —NHS(O)₂CH₃.

In one embodiment, the present invention provides novel compounds of structural Formula (VI):

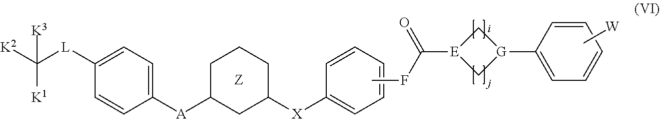

or a pharmaceutically acceptable salt thereof,
wherein:
Z is selected from

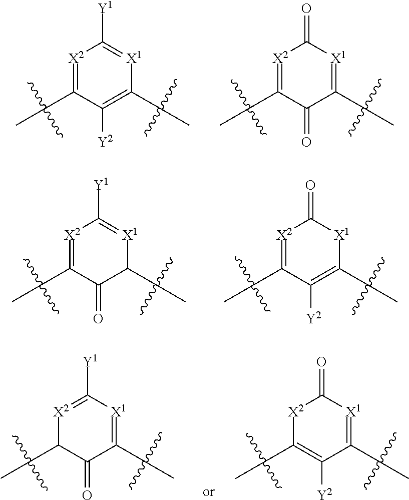

X¹ and X² are each independently N or CH;
A is —NH—, —N(CH₃)—, —N(OH)—, —N(OCH₃)—, —NHC(O)—, —OC(O)NH—, —NHC(O)NH—, —NHS(O)₂—, —NHS(O)₂NH—, —S—, —O—, or —CH₂—, C(O)—, —S(O)—, or —S(O)₂—;
X is absent, —NH—, —N(CH₃)—, —N(OH)—, —N(OCH₃)—, —C(O)—, —SO₂—, —NHC(O)—, —NH(CO)NH—, —NHS(O)₂—, —NHS(O)₂NH—, —CH₂—, —SO—, a benzoyl moiety, or a phenyl moiety,
E is CH or N;
F is absent, —O—, —NH—, or —N(OH)—;
G is N or CH;
W is —CH₃, —CF₃, —OCF₃, OCH₂CF₃, or —CH₂CH₃;
Y¹ and Y² are each independently hydrogen, halogen, C₁₋₁₆alkyl, OH, —O—C₁₋₁₆alkyl, —S—C₁₋₁₆alkyl, —S(O)—C₁₋₁₆alkyl, —S(O₂)—C₁₋₁₆alkyl, —C(O)—C₁₋₁₆alkyl, a cycloalkyl group, a heterocyclic group, a heteroaromatic group, or an aryl group, wherein the —C₁₋₁₆alkyl, —O—C₁₋₁₆alkyl, cycloalkyl group, S—C₁₋₁₆alkyl, —S(O)—C₁₋₁₆alkyl, —S(O₂)—C₁₋₁₆alkyl, —C(O)—C₁₋₁₆alkyl, heterocyclic group, heteroaromatic group, and aryl group are optionally substituted with one or more substituents independently selected from halogen, hydroxyl, oxo, lower alkyl, lower alkoxyl, and phenyl;
R³ is hydrogen, —C₁₋₁₆alkyl, —O—C₁₋₁₆alkyl, —S—C₁₋₁₆alkyl, —S(O)—C₁₋₁₆alkyl, —S(O)₂—C₁₋₁₆ alkyl, —C(O)—C₁₋₁₆alkyl, a cycloalkyl group, a heterocyclic group, a heteroaromatic group, or an aryl group, wherein the —C₁₋₁₆alkyl, —O—C₁₋₁₆alkyl, cycloalkyl group, S—C₁₋₁₆alkyl, —S(O)—C₁₋₁₆ alkyl, —S(O)₂—C₁₋₁₆alkyl, —C(O)—C₁₋₁₆alkyl, heterocyclic group, heteroaromatic group, and aryl group are optionally substituted with one or more substituents independently selected from halogen, hydroxyl, oxo, lower alkyl, lower alkoxyl, halogenated lower alkyl, halogenated lower alkoxy, and phenyl;
i is 1, 2, 3 or 4;
j is 1, 2, 3 or 4;
L is —NH—, —NHC(O)—, —S—, —O—, —CH₂—, C(O)—, —S(O)—, or —S(O₂)—, or —NHS(O)₂—;
K¹, K² and K³ are each independently —F, —C, —Br, —I, —CH₃, OH, CF₃, —NHS(O)₂CF₃, NHS(O)₂CH₃.

In one embodiment, in compounds of structural Formulas (I), (II), (III) or (IV), B is NR².

In one embodiment, in compounds of structural Formulas (I), (II), (III) or (IV), R² is YR³, wherein Y is absent, —C(O)—, or —SO₂—, and R³ is an aryl group optionally substituted with one or more substituents independently selected from halogen, hydroxyl, lower alkyl, lower alkoxyl, halogenated lower alkyl, and phenyl.

In one embodiment, in compounds of structural Formulas (I), (II), (III), (IV), (V) or (VI), R¹ is CF₃ and n is 1.

In one embodiment, in compounds of structural Formulas (I), (II), (III) or (IV), (V) or (VI), A is NH.

In one embodiment, in compounds of structural Formulas (I), (II), (III) or (IV), (V) or (VI), X¹ and X² are each N.

In one embodiment, in compounds of structural Formulas (I), (II), (III) or (IV), (V) or (VI), Y¹ and Y² are each H.

Exemplary compounds falling within the scope of the present invention include, but are not limited to:

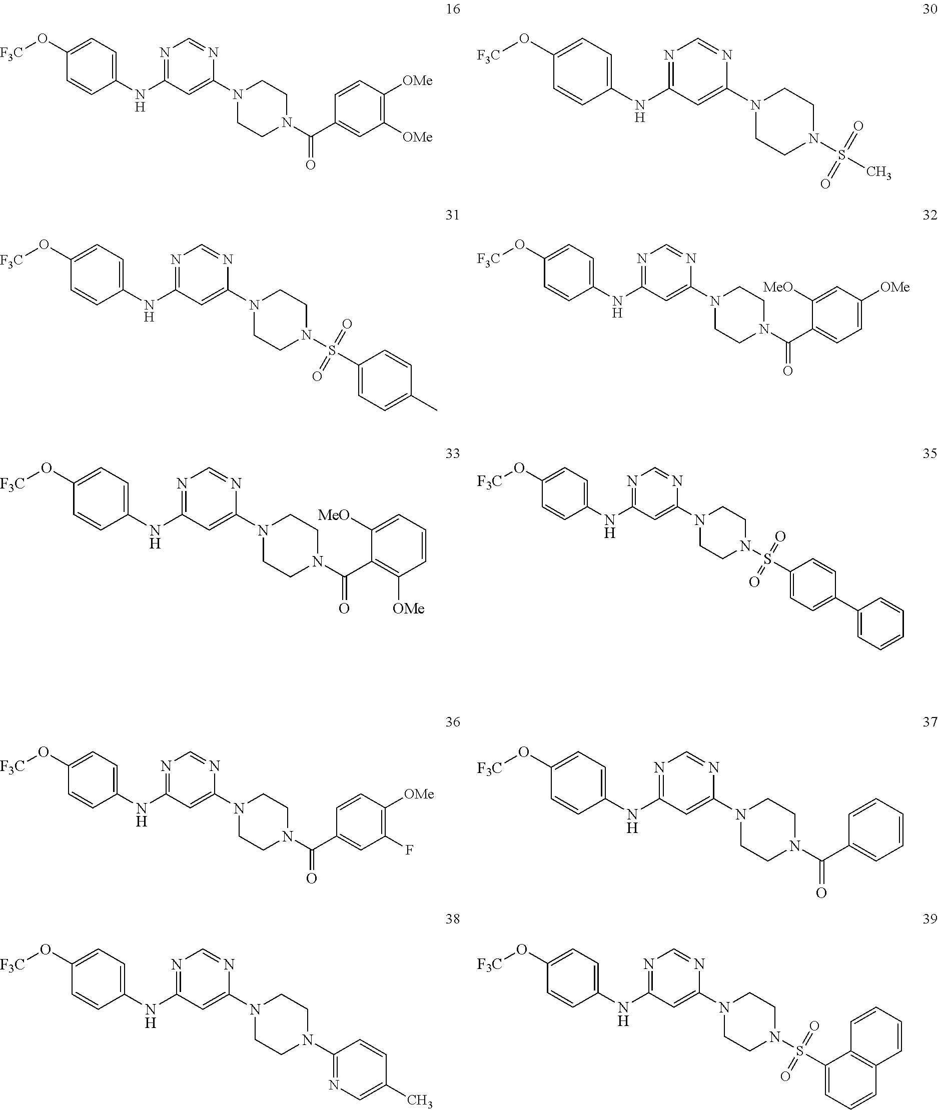

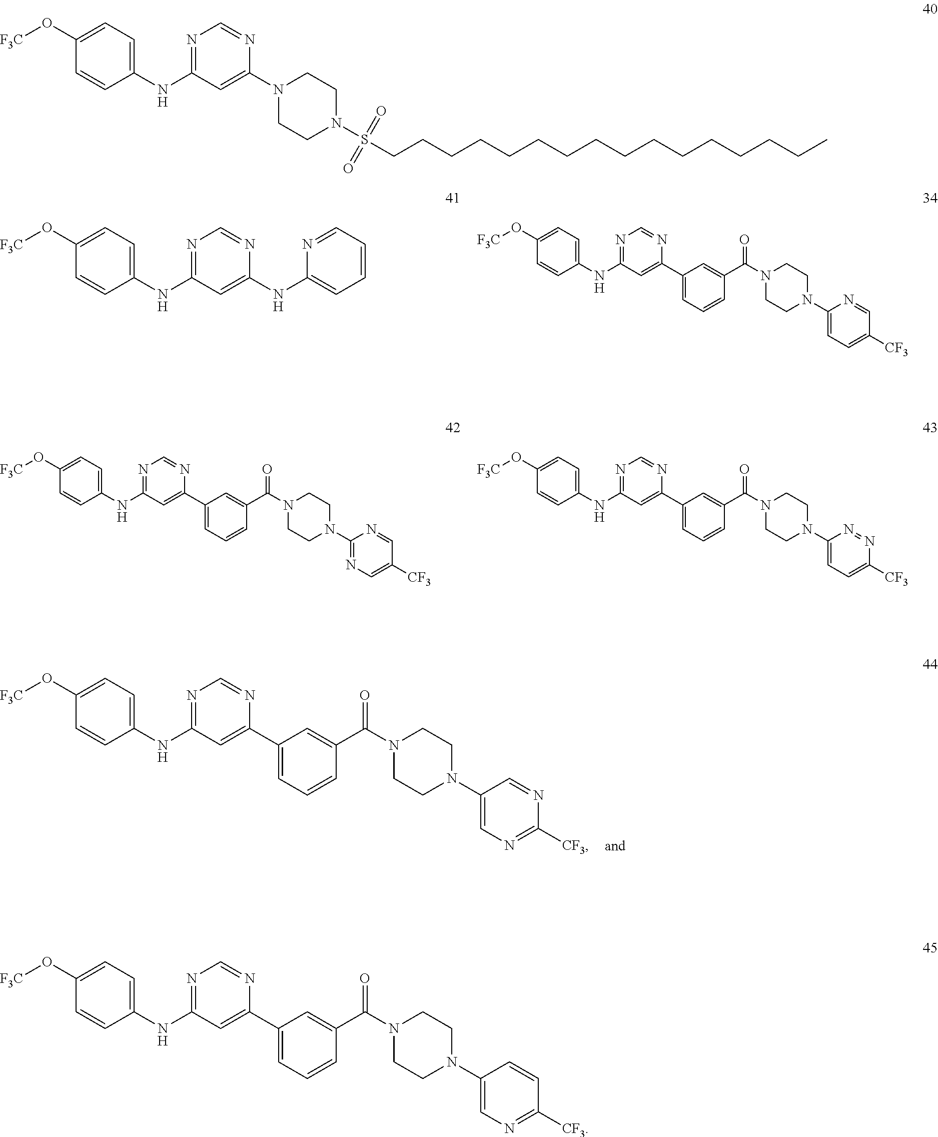

In preferred embodiments of the invention, the compounds incorporate solubilizing moieties to increase solubility in aqueous solutions. Increased aqueous solubility can be expected to provide therapeutic agents having increased bioavailability. Solubilizing moieties that may be incorporated into the compounds of the present invention include moieties having, for example, multiple hydrogen bonding sites, positively charged moieties, and/or negatively charged moieties.

Increased aqueous solubility can also facilitate the preparation of pharmaceutical formulations.

The present invention also includes novel methods of treating or preventing cancer, or overcoming cancer resistance, using the compounds of the invention. In one embodiment, the cancer is selected from the group consisting of breast cancer, Breast Invasive Carcinoma, Uterine Corpus Endometrioid Carcinoma, Ovarian Serous Cystadenocarcinoma chronic myelogenous leukemia, acute lymphoblastic leukemia, childhood B-cell acute lymphocytic leukemia (B-ALL), neutrophilic-CML, osteosarcoma, glioblastoma, cervical cancer, lung cancer, colon cancer, melanoma, ovarian cancer, prostate cancer, liver cancer, pancreatic cancer, CNS tumors (including brain tumors), neuroblastoma, leukemia, bone cancer, intestinal cancer, lymphoma, chronic myeloproliferative disorders (MPD, also known as myeloproliferative neoplasms/disorders (MPN) like Polycythemia vera (PV), Essential Thrombocythemia (ET), Myelofibrosis (MF), Thrombocythemia, Chronic neutrophilic leukemia, Eosinophilia, and combinations thereof.

The present invention also includes combination therapies for treatment or prevention of cancer, or overcoming cancer resistance, comprising administration of a compound of the present invention in combination with a known chemotherapeutic agent. In one embodiment, the known chemotherapeutic agent is selected from the group consisting of imatinib, nilotinib, dasatinib, bosutinib, ponatinib, bafetinib, rebastinib, tozasertib and danusertib.

As used herein, the term “about” refers to a +/−10% variation from the nominal value. It is to be understood that such a variation is always included in a given value provided herein, whether or not it is specifically referred to.

Unless defined otherwise, all technical and scientific terms used herein have the same meaning as commonly understood by one of ordinary skill in the art to which this invention belongs.

The articles “a” and “an” are used herein to refer to one or to more than one (i.e., to at least one) of the grammatical object of the article. By way of example, “an element” means one element or more than one element.

The term “ALL” is an abbreviation of acute lymphoblastic leukemia and the term “CML” is an abbreviation of chronic myeloid leukemia.

The term “Ph chromosome” refers to the Philadelphia chromosome, which is a specific genetic abnormality involving chromosome 22 of leukemia cancer cells (particularly chronic myeloid leukemia (CML)). This chromosome is defective and unusually short because of reciprocal translocation, t(9; 22)(q34; q11), of genetic material between chromosome 9 and chromosome 22, and contains a fusion gene called BCR-ABL1. This gene resulted from a reciprocal translocation of Abelson (ABL) gene of chromosome 9 juxtaposed onto the breakpoint cluster region BCR gene of chromosome 22, coding for a hybrid oncoprotein consists of the tyrosine kinase signaling protein (ABL) that is constitutively active (“always on”), causing the cell to divide uncontrollably by interrupting the stability of the genome and impairing various signaling pathways governing the cell cycle. Members of ABL family are nonreceptor tyrosine kinases, ABL1 and ABL2, which transduce a wide range of extracellular signals to protein networks that control proliferation, survival, migration, and invasion.

The term “abnormal,” when used in the context of organisms, tissues, cells or components thereof, refers to those organisms, tissues, cells or components thereof that differ in at least one observable or detectable characteristic (e.g., age, treatment, time of day, etc.) from those organisms, tissues, cells or components thereof that display the “normal” (expected) respective characteristic. Characteristics that are normal or expected for one cell or tissue type might be abnormal for a different cell or tissue type.

A “disease” is a state of health of a subject wherein the subject cannot maintain homeostasis, and wherein if the disease is not ameliorated then the human health continues to deteriorate. In contrast, a “disorder” in a subject is a state of health in which the subject is able to maintain homeostasis, but in which the subject's state of health is less favorable than it would be in the absence of the disorder. Left untreated, a disorder does not necessarily cause a further decrease in the subject's state of health.

A disease or disorder is “alleviated” if the severity of a sign or symptom of the disease or disorder, the frequency with which such a sign or symptom is experienced by a patient, or both, is reduced.

The terms “patient,” “subject,” or “individual” are used interchangeably herein, and refer to any animal, or cells thereof whether in vitro or in situ, amenable to the methods described herein. In a non-limiting embodiment, the patient, subject or individual is a human.

As used herein, the term “pharmaceutical composition” refers to a mixture of at least one compound useful within the invention with a pharmaceutically acceptable carrier. The pharmaceutical composition facilitates administration of the compound to a patient or subject. Multiple techniques of administering a compound exist in the art including, but not limited to, intravenous, oral, aerosol, parenteral, ophthalmic, pulmonary and topical administration.

A “therapeutic” treatment is a treatment administered to a subject who exhibits signs of pathology, for the purpose of diminishing or eliminating those signs.

As used herein, the term “treatment” or “treating” is defined as the application or administration of a therapeutic agent, i.e., a compound of the invention (alone or in combination with another pharmaceutical agent), to a patient, or application or administration of a therapeutic agent to an isolated tissue or cell line from a patient (e.g., for diagnosis or ex vivo applications), who has a condition contemplated herein, a sign or symptom of a condition contemplated herein or the potential to develop a condition contemplated herein, with the purpose to cure, heal, alleviate, relieve, alter, remedy, ameliorate, improve or affect a condition contemplated herein, the symptoms of a condition contemplated herein or the potential to develop a condition contemplated herein. Such treatments may be specifically tailored or modified, based on knowledge obtained from the field of pharmacogenomics.

As used herein, the terms “effective amount,” “pharmaceutically effective amount” and “therapeutically effective amount” refer to a nontoxic but sufficient amount of an agent to provide the desired biological result. That result may be reduction and/or alleviation of a sign, a symptom, or a cause of a disease or disorder, or any other desired alteration of a biological system. An appropriate therapeutic amount in any individual case may be determined by one of ordinary skill in the art using routine experimentation.

As used herein, the term “pharmaceutically acceptable” refers to a material, such as a carrier or diluent, which does not abrogate the biological activity or properties of the compound, and is relatively non-toxic, i.e., the material may be administered to an individual without causing an undesirable biological effect or interacting in a deleterious manner with any of the components of the composition in which it is contained.

As used herein, the language “pharmaceutically acceptable salt” refers to a salt of the administered compound prepared from pharmaceutically acceptable non-toxic acids, including inorganic acids, organic acids, solvates, hydrates, or clathrates thereof. Examples of such inorganic acids are hydrochloric, hydrobromic, hydroiodic, nitric, sulfuric, phosphoric, hexafluorophosphoric, and the like. Appropriate organic acids may be selected, for example, from aliphatic, aromatic, carboxylic and sulfonic classes of organic acids, examples of which are formic, acetic, propionic, succinic, camphorsulfonic, citric, fumaric, gluconic, isethionic, lactic, malic, mucic, tartaric, para-toluenesulfonic, glycolic, glucuronic, maleic, furoic, glutamic, benzoic, anthranilic, salicylic, phenylacetic, mandelic, embonic (pamoic), ethanesulfonic, pantothenic, benzenesulfonic (besylate), stearic, sulfanilic, alginic, galacturonic, kojic acid, methanesulfonic (mesylate), butyric, sulfosalicylic, pyruvic, isocitric, shikimic, oxalic, suberic, malonic, lauric, amsonic, azelaic, and the like. Furthermore, pharmaceutically acceptable salts include, by way of non-limiting example, alkaline earth metal salts (e.g., calcium or magnesium), alkali metal salts (e.g., sodium-dependent or potassium), and ammonium salts.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the invention within or to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the invention, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound useful within the invention, and are physiologically acceptable to the patient. Supplementary active compounds may also be incorporated into the compositions. The “pharmaceutically acceptable carrier” may further include a pharmaceutically acceptable salt of the compound useful within the invention. Other additional ingredients that may be included in the pharmaceutical compositions used in the practice of the invention are known in the art and described, for example in Remington's Pharmaceutical Sciences (Genaro, Ed., Mack Publishing Co., 1985, Easton, Pa.), which is incorporated herein by reference.

An “effective amount” of a delivery vehicle is that amount sufficient to effectively bind or deliver a compound.

As used herein, the term “alkyl,” by itself or as part of another substituent means, unless otherwise stated, refers to a straight or branched chain hydrocarbon having the number of carbon atoms designated (e.g. C1-10 means one to ten carbon atoms) and including straight, branched chain, or cyclic substituent groups. Examples include methyl, ethyl, propyl, isopropyl, butyl, isobutyl, tert-butyl, pentyl, neopentyl, hexyl, and cyclopropylmethyl.

As used herein, the term “lower alkyl”, employed alone or in combination with other groups, refers to a branched or straight-chain alkyl radical of one to nine carbon atoms, in another embodiment one to six carbon atoms, in a further embodiment one to four carbon atoms. This term is further exemplified by radicals such as methyl, ethyl, n-propyl, isopropyl, n-butyl, s-butyl, isobutyl, t-butyl, n-pentyl, 3-methylbutyl, n-hexyl, 2-ethylbutyl and the like.

As used herein, the term “substituted alkyl” means alkyl as defined above, substituted by one, two or three substituents selected from the group consisting of halogen, —OH, alkoxy, —NH₂, amino, azido, —N(CH₃)₂, —C(O)OH, trifluoromethyl, —C(O)O(C₁-C₄) alkyl, —C(O)NH₂, —SO₂NH₂, —C(═NH)NH₂, and —NO₂. Examples of substituted alkyls include, but are not limited to, 2,2-difluoropropyl, 2-carboxycyclopentyl and 3-chloropropyl.

As used herein, the term “alkoxy” employed alone or in combination with other terms means, unless otherwise stated, an alkyl group having the designated number of carbon atoms, as defined above, connected to the rest of the molecule via an oxygen atom, such as, for example, methoxy, ethoxy, 1-propoxy, 2-propoxy (isopropoxy) and the higher homologs and isomers.

As used herein, the term “halo” or “halogen”, employed alone or as part of another substituent, means, unless otherwise stated, a fluorine, chlorine, bromine, or iodine atom.

As used herein, the term “cycloalkyl” refers to a monocyclic or polycyclic non-aromatic radical, wherein each of the atoms forming the ring (i.e., skeletal atoms) is a carbon atom. In one embodiment, the cycloalkyl group is saturated or partially unsaturated. In another embodiment, the cycloalkyl group is fused with an aromatic ring. Cycloalkyl groups include groups having from 3 to 10 ring atoms. Illustrative examples of cycloalkyl groups include, but are not limited to: monocyclic cycloalkyls such as cyclopropyl, cyclobutyl, cyclopentyl, cyclohexyl, cycloheptyl, and cyclooctyl; dicyclic cycloalkyls such as tetrahydronaphthyl, indanyl, and tetrahydropentalene; and polycyclic cycloalkyls such as adamantine and norbornane.

As used herein, the term “heterocycloalkyl” or “heterocyclyl” refers to a heteroalicyclic group containing one to four ring heteroatoms each selected from O, S and N. In one embodiment, each heterocycloalkyl group has from 4 to 10 atoms in its ring system, with the proviso that the ring of said group does not contain two adjacent O or S atoms. In another embodiment, the heterocycloalkyl group is fused with an aromatic ring. In one embodiment, the nitrogen and sulfur heteroatoms may be optionally oxidized, and the nitrogen atom may be optionally quaternized. The heterocyclic system may be attached, unless otherwise stated, at any heteroatom or carbon atom that affords a stable structure. A heterocycle may be aromatic or non-aromatic in nature. In one embodiment, the heterocycle is a heteroaryl.

An example of a 3-membered heterocycloalkyl group includes, and is not limited to, aziridine. Examples of 4-membered heterocycloalkyl groups include, and are not limited to, azetidine and a beta lactam. Examples of 5-membered heterocycloalkyl groups include, and are not limited to, pyrrolidine, oxazolidine and thiazolidinedione. Examples of 6-membered heterocycloalkyl groups include, and are not limited to, piperidine, morpholine and piperazine. Other non-limiting examples of heterocycloalkyl groups include: non-aromatic heterocycles such as monocyclic groups such as aziridine, oxirane, thiirane, azetidine, oxetane, thietane, pyrrolidine, pyrroline, pyrazolidine, imidazoline, dioxolane, sulfolane, 2,3-dihydrofuran, 2,5-dihydrofuran, tetrahydrofuran, thiophane, piperidine, 1,2,3,6-tetrahydropyridine, 1,4-dihydropyridine, piperazine, morpholine, thiomorpholine, pyran, 2,3-dihydropyran, tetrahydropyran, 1,4-dioxane, 1,3-dioxane, homopiperazine, piperazinone, piperazin-dione, homopiperidine, 1,3-dioxepane, 4,7-dihydro-1,3-dioxepin, and hexamethyleneoxide.

As used herein, the term “aromatic” refers to a carbocycle or heterocycle with one or more polyunsaturated rings and having aromatic character, i.e., having (4n+2) delocalized π (pi) electrons, where n is an integer.

As used herein, the term “aryl,” employed alone or in combination with other terms, means, unless otherwise stated, a carbocyclic aromatic system containing one or more rings (typically one, two or three rings), wherein such rings may be attached together in a pendent manner, such as a biphenyl, or may be fused, such as naphthalene. Examples of aryl groups include phenyl, anthracyl, and naphthyl.

As used herein, the term “heteroaryl” or “heteroaromatic” refers to a heterocycle having aromatic character. A polycyclic heteroaryl may include one or more rings that are partially saturated. Examples of heteroaryl groups include, but are not limited to, the following: pyridyl, pyrazinyl, pyrimidinyl (particularly 2-, 4- and 6-pyrimidinyl), pyridazinyl, thienyl, furyl, pyrrolyl (particularly 2-pyrrolyl), imidazolyl, thiazolyl, oxazolyl, pyrazolyl (particularly 3- and 5-pyrazolyl), isothiazolyl, 1,2,3-triazolyl, 1,2,4-triazolyl, 1,3,4-triazolyl, tetrazolyl, 1,2,3-thiadiazolyl, 1,2,3-oxadiazolyl, 1,3,4-thiadiazolyl and 1,3,4-oxadiazolyl.

Examples of polycyclic heterocycles and heteroaryls include indolyl (particularly 3-, 4-, 5-, 6- and 7-indolyl), indolinyl, quinolyl, tetrahydroquinolyl, isoquinolyl (particularly 1- and 5-isoquinolyl), 1,2,3,4-tetrahydroisoquinolyl, cinnolinyl, quinoxalinyl (particularly 2- and 5-quinoxalinyl), quinazolinyl, phthalazinyl, 1,8-naphthyridinyl, 1,4-benzodioxanyl, coumarin, dihydrocoumarin, 1,5-naphthyridinyl, benzofuryl (particularly 3-, 4-, 5-, 6- and 7-benzofuryl), 2,3-dihydrobenzofuryl, 1,2-benzisoxazolyl, benzothienyl (particularly 3-, 4-, 5-, 6-, and 7-benzothienyl), benzoxazolyl, benzothiazolyl (particularly 2-benzothiazolyl and 5-benzothiazolyl), purinyl, benzimidazolyl (particularly 2-benzimidazolyl), benzotriazolyl, thioxanthinyl, carbazolyl, carbolinyl, acridinyl, pyrrolizidinyl, and quinolizidinyl, benoxazole, benzimidazole, quinoline, isoquinaline.

As used herein, the term “substituted” means that an atom or group of atoms has replaced hydrogen as the substituent attached to another group. The term “substituted” further refers to any level of substitution, namely mono-, di-, tri-, tetra-, or penta-substitution, where such substitution is permitted. The substituents are independently selected, and substitution may be at any chemically accessible position. In one embodiment, the substituents vary in number between one and four. In another embodiment, the substituents vary in number between one and three. In yet another embodiment, the substituents vary in number between one and two.

As used herein, the term “optionally substituted” means that the referenced group may be substituted or unsubstituted. In one embodiment, the referenced group is optionally substituted with zero substituents, i.e., the referenced group is unsubstituted. In another embodiment, the referenced group is optionally substituted with one or more additional group(s) individually and independently selected from groups described herein.

In one embodiment, the substituents are independently selected from the group consisting of oxo, halogen, —CN, —NH₂, —OH, —NH(CH₃), —N(CH₃)₂, alkyl (including straight chain, branched and/or unsaturated alkyl), substituted or unsubstituted cycloalkyl, substituted or unsubstituted heterocycloalkyl, fluoro alkyl, substituted or unsubstituted heteroalkyl, substituted or unsubstituted alkoxy, fluoroalkoxy, —S— alkyl, —S(O)₂alkyl, —C(O)NH[substituted or unsubstituted alkyl, or substituted or unsubstituted phenyl], —C(O)N[H or alkyl]₂, —OC(O)N[substituted or unsubstituted alkyl]₂, —NHC(O)NH[substituted or unsubstituted alkyl, or substituted or unsubstituted phenyl], —NHC(O)alkyl, —N[substituted or unsubstituted alkyl]C(O)[substituted or unsubstituted alkyl], —NHC(O)[substituted or unsubstituted alkyl], —C(OH)[substituted or unsubstituted alkyl]₂, and —C(NH₂)[substituted or unsubstituted alkyl]₂. In another embodiment, by way of example, an optional substituent is selected from oxo, fluorine, chlorine, bromine, iodine, —CN, —NH₂, —OH, —NH(CH₃), —N(CH₃)₂, —CH₃, —CH₂CH₃, —CH(CH₃)₂, —CF₃, —CH₂CF₃, —OCH₃, —OCHYCH₃, —OCH(CH₃)₂, —OCF₃, —OCH₂CF₃, —S(O)₂—CH₃, —C(O)NH₂, —C(═O)—NHCH₃, —NHC(O)NHCH₃, —C(O)CH₃, —ON(O)₂, and —C(O)OH. In yet one embodiment, the substituents are independently selected from the group consisting of C₁₋₆ alkyl, —OH, C₁₋₆ alkoxy, halo, amino, acetamido, oxo and nitro. In yet another embodiment, the substituents are independently selected from the group consisting of C₁₋₆ alkyl, C₁₋₆ alkoxy, halo, acetamido, and nitro. As used herein, where a substituent is an alkyl or alkoxy group, the carbon chain may be branched, straight or cyclic, with straight being preferred.

The present invention also provides pharmaceutical compositions comprising a compound in accordance with the present invention, and a pharmaceutically acceptable carrier or diluent.

The pharmaceutical compositions of the present invention may be administered orally, topically, parenterally, by inhalation or spray or rectally in dosage unit formulations containing conventional non-toxic pharmaceutically acceptable carriers, adjuvants and vehicles. The term parenteral as used herein includes subcutaneous injections, intravenous, intramuscular, intrasternal injection or infusion techniques.

The pharmaceutical compositions may be in a form suitable for oral use, for example, as tablets, troches, lozenges, aqueous or oily suspensions, dispersible powders or granules, emulsion hard or soft capsules, or syrups or elixirs. Compositions intended for oral use may be prepared according to methods known to the art for the manufacture of pharmaceutical compositions and may contain one or more agents selected from the group of sweetening agents, flavouring agents, colouring agents and preserving agents in order to provide pharmaceutically elegant and palatable preparations. Tablets contain the active ingredient in admixture with suitable non-toxic pharmaceutically acceptable excipients including, for example, inert diluents, such as calcium carbonate, sodium carbonate, lactose, calcium phosphate or sodium phosphate; granulating and disintegrating agents, such as corn starch, or alginic acid; binding agents, such as starch, gelatine or acacia, and lubricating agents, such as magnesium stearate, stearic acid or talc. The tablets can be uncoated, or they may be coated by known techniques in order to delay disintegration and absorption in the gastrointestinal tract and thereby provide a sustained action over a longer period. For example, a time delay material such as glyceryl monostearate or glyceryl distearate may be employed.

Pharmaceutical compositions for oral use may also be presented as hard gelatine capsules wherein the active ingredient is mixed with an inert solid diluent, for example, calcium carbonate, calcium phosphate or kaolin, or as soft gelatine capsules wherein the active ingredient is mixed with water or an oil medium such as peanut oil, liquid paraffin or olive oil.

Aqueous suspensions contain the active compound in admixture with suitable excipients including, for example, suspending agents, such as sodium carboxymethylcellulose, methyl cellulose, hydropropylmethylcellulose, sodium alginate, polyvinylpyrrolidone, gum tragacanth and gum acacia; dispersing or wetting agents such as a naturally-occurring phosphatide, for example, lecithin, or condensation products of an alkylene oxide with fatty acids, for example, polyoxyethyene stearate, or condensation products of ethylene oxide with long chain aliphatic alcohols, for example, hepta-decaethyleneoxycetanol, or condensation products of ethylene oxide with partial esters derived from fatty acids and a hexitol for example, polyoxyethylene sorbitol monooleate, or condensation products of ethylene oxide with partial esters derived from fatty acids and hexitol anhydrides, for example, polyethylene sorbitan monooleate. The aqueous suspensions may also contain one or more preservatives, for example ethyl, or n-propyl p-hydroxy-benzoate, one or more colouring agents, one or more flavouring agents or one or more sweetening agents, such as sucrose or saccharin.

Oily suspensions may be formulated by suspending the active ingredients in a vegetable oil, for example, arachis oil, olive oil, sesame oil or coconut oil, or in a mineral oil such as liquid paraffin. The oily suspensions may contain a thickening agent, for example, beeswax, hard paraffin or cetyl alcohol. Sweetening agents such as those set forth above, and/or flavouring agents may be added to provide palatable oral preparations. These compositions can be preserved by the addition of an anti-oxidant such as ascorbic acid.

Dispersible powders and granules suitable for preparation of an aqueous suspension by the addition of water provide the active compound in admixture with a dispersing or wetting agent, suspending agent and one or more preservatives. Suitable dispersing or wetting agents and suspending agents are exemplified by those already mentioned above. Additional excipients, for example sweetening, flavouring and colouring agents, may also be present.

Pharmaceutical compositions of the invention may also be in the form of oil-in-water emulsions. The oil phase may be a vegetable oil, for example, olive oil or arachis oil, or a mineral oil, for example, liquid paraffin, or it may be a mixture of these oils. Suitable emulsifying agents may be naturally-occurring gums, for example, gum acacia or gum tragacanth; naturally-occurring phosphatides, for example, soy bean, lecithin; or esters or partial esters derived from fatty acids and hexitol, anhydrides, for example, sorbitan monoleate, and condensation products of the said partial esters with ethylene oxide, for example, polyoxyethylene sorbitan monoleate. The emulsions may also contain sweetening and flavouring agents.

Syrups and elixirs may be formulated with sweetening agents, for example, glycerol, propylene glycol, sorbitol or sucrose. Such formulations may also contain a demulcent, a preservative, and/or flavouring and colouring agents.

The pharmaceutical compositions may be in the form of a sterile injectable aqueous or oleaginous suspension. This suspension may be formulated according to known art using suitable dispersing or wetting agents and suspending agents such as those mentioned above. The sterile injectable preparation may also be sterile injectable solution or suspension in a non-toxic parentally acceptable diluent or solvent, for example, as a solution in 1,3-butanediol. Acceptable vehicles and solvents that may be employed include, but are not limited to, water, Ringer's solution, lactated Ringer's solution and isotonic sodium chloride solution. Other examples are, sterile, fixed oils which are conventionally employed as a solvent or suspending medium, and a variety of bland fixed oils including, for example, synthetic mono- or diglycerides. In addition, fatty acids such as oleic acid find use in the preparation of injectables.

Other pharmaceutical compositions and methods of preparing pharmaceutical compositions are known in the art and are described, for example, in “Remington: The Science and Practice of Pharmacy,” Gennaro, A., Lippincott, Williams & Wilkins, Philadelphia, PA (2000) (formerly “Remingtons Pharmaceutical Sciences”).

The invention will now be described with reference to specific examples. It will be understood that the following examples are intended to describe embodiments of the invention and are not intended to limit the invention in any way.

### Examples

### Experimental:

Each tested compound was dissolved in a suitable vehicle and cells were exposed to successive solutions containing an increasing concentration of each test compound, as well as a control sample containing no test compound. A range of 5 (or 10) concentrations were tested in triplicates starting from 1 μM. The potency of each test compound was assessed by cell viability using an XTT assay [53, 54]. The ratio of cell viability (CV %) was determined by calculating the average of each triplicate, normalizing to the average in case to the average of the control sample.

Dose-concentration (D/C) relationship was plotted using Microsoft excel. Standard deviation (STD) was calculated for each triplicate and the curve logarithmic equation was calculated for each curve. IC₅₀ was calculated by solving the reverse natural logarithm for y=50.

### Cell Lines, Transduced Cells and Patient-Derived Long-Term Cultures (PD-LTCs)

1. Ba/F3 cells were obtained from the German Collection of Microorganisms and Cell Cultures (DSMZ, Braunschweig, Germany) and maintained in RPMI 1640 medium supplemented with 10% fetal calf serum (Invitrogen) containing 10 ng/ml of interleukin-3 (Cell Concepts, Umkirch, Germany). Ecotropic Phoenix cells and Rat-1 cells were cultured in Dulbecco's modified Eagle's medium supplemented with 10% fetal calf serum. GNF-2 (Sigma-Adrich, Steinheim, Germany) was dissolved in dimethyl sulfoxide and added at a final concentration of 2 mM. Cell growth was assessed by dye exclusion using Trypan blue. Proliferation was assessed using the XTT proliferation kit (Roche, Mannheim, Germany), according to the manufacturer's instructions [54].
2. Ba/F3: IL-3 dependent lymphatic murine pro B cell line. Ba/F3 cells expressing either p185-BCR-ABL1 (p185-BCR-ABL-Ba/F3) or p210-BCR-ABL (p210-BCR-ABL-Ba/F3). p185-BCR-ABL1 (activated kinases substitute for the IL-3 signaling and therefore render the cells IL3 independent. Ba/F3 cells expressing either p185-BCR-ABL (p185-Ba/F3) or p210-BCR-ABL (p210-Ba/F3). Ba/F3 cells obtained from the German Collection of Microorganisms and Cell Cultures (DSMZ, Braunschweig, Germany).
3. Mutant transduced Ba/F3 cells (T315I-p185-BCR-ABL1 or T315I-p210-BCR-ABL1): Ba/F3 cells expressing either the mutated forms T315I-p185-BCR-ABL1 or T315I-p210-BCR-ABL1.
4. WT-Sup-B15: A Ph+ ALL cell line harboring the p185-BCR-ABL. Sup-B15: established from the bone marrow of a 9-year-old boy with acute lymphoblastic leukemia (B cell precursor ALL) in second relapse in 1984; described in the literature to carry the ALL-variant (m-BCR) of the BCR-ABL1 fusion gene (e1-a2) correct, because it results in the p185-BCR-ABL version.
5. RT-Sup-B15: Imatinib resistant Ph+ ALL. Cultured in increasing amounts of Imatinib.
6. V617FJAK2 HEL: (Human erythroleukemia) is a growth factor independent erythroleukemic cell line established from the bone marrow of a patient with relapsed Hodgkin disease after autologous bone marrow transplantation (Martin P & Papayannopoulou T: Science 1982; 216:1233-1235). HEL cells display a block in differentiation at the level of common erythroid-megakaryocytic progenitor and have been commonly used as a model to study erythroid and megakaryocytic differentiation [55].
7. Jurkat: an immortalized human T lymphocyte first derived from the peripheral blood of a child suffering from T cell leukemia. Jurkat cells are used to study acute T cell leukemia, T cell signaling, and the expression of various chemokine receptors susceptible to viral entry, particularly HIV.

The retroviral vector PINCO was used for the transduction of different BCR-ABL constructs in Ba/F3 cells. When transduced with the empty vector as a control, it can be used to determine that factor independency is not due to the retroviral vector.

### Retrovirus-Based Mutagenesis Screen [56]

For a modified retrovirus-based mutagenesis screen, Ba/F3 cells were retrovirally transduced with either p185^{BCR/ABL} or its resistance mutants and selected by interleukin (IL)-3 withdrawal. A perfectly balanced pool of 107 cells was cultured with increasing concentrations of corresponding compound (0, 10, 50, 100, 500 and 1000 nM). After 28 days clones were obtained by limiting dilution in 96-well plates. Genomic DNA for sequencing the BCR ABL kinase domain was extracted using QIAamp DNA Mini Kit (Qiagen, Düsseldorf, Germany). For amplification the following primers were used: ALL-TB 5′-GCAAGACCGGGCAGATCT-3′ and R-ABL-A 5′-GTTGCACTCCCTCAGGTAGTC-3′. PCR products were sequenced by Seqlab (Göttingen, Germany) using the AN4 5′-TGGTTCATCATCATTCAACGGTGG-3′. The sequence data were analyzed for mutations with Clone Manager Professional (Sci ED Software, Morrison, NC, USA).
a. The Ba/F3 were obtained from the German Collection of Microorganisms and Cell Cultures (DSMZ, Braunschweig, Germany) and were maintained as previously described [57].
b. Ph+ ALL patient derived long term cultures (PD-LTCs) expressing T315I-BCR-ABL (KÖ) were obtained from a patient enrolled in the German Multi-Center Study Group for acute lymphatic leukemia of the adult (GMALL 07/2003) upon informed and written consent [58] and were maintained in a serum-free medium consisting of IMDM supplemented with 1 mg/mL of bovine insulin, 5×10⁵ M β-mercaptoethanol (Sigma, Steinheim, Germany), 200 mg/mL Fe—saturated human apo-transferrin (Invitrogen, Karlsruhe, Germany), 0.6% human serum albumin (Sanquin, Amsterdam, The Netherlands), 2.0 mM L-glutamine and 20 mg/mL cholesterol (Sigma) [59]. Proliferation was assessed with the XTT proliferation kit (Roche, Mannheim, Germany) according to the manufacturer's instructions [53].

Each compound was assayed against a panel of Patient-Derived Long-Term cultures (PD-LTCs) including:
8. BV173: p210-BCR-ABL, Lymphatic Blast Crisis CML leukemia (CML can develop in ˜70% a myeloid blast crisis [K562] and in ˜30% a lymphatic blast crisis [BV173]). CML in myeloid blast crisis, resistant to known AKIs but has normal BCR-ABL1.
9. PH: primary Patient-Derived Long-Term Cultures (PD-LTCs) from Ph⁺ ALL patients Ph+ ALL (p185-BCR-ABL) that are fully responsive to TKIs ˜30% of adult ALL patients harbor the Ph chromosome, that represent a high-risk group of ALL.
10. BV: PD-LTCs from Ph⁺ ALL patients. Ph⁺ ALL patients. We selected two different PD-LTCs: one, the PH, fully responsive to TKIs and one, BV, exhibiting a nearly complete resistance to TKIs not attributable to mutations in the TKD.
11. HP: PD-LTCs derived from Ph− ALL patient (not harboring the Ph chromosome). HP was used as negative control.
12. VM: derived from a Ph+ ALL patient harboring p210-BCR-ABL.
13. KÖ (Ph+T315I-p185-BCR-ABL ALL): PD-LTCs derived from a Ph+ ALL patient harboring the T315I-p185-BCR-ABL. T315I is a gatekeeper mutation that confers resistance to all first and second generation ABL Kinase Inhibitors (AKIs). AKI-resistant cells are responsive only to ponatinib or PF114. Ponatinib approved as active against patients harboring this mutation.

### Example 1: Assays of Compound 16

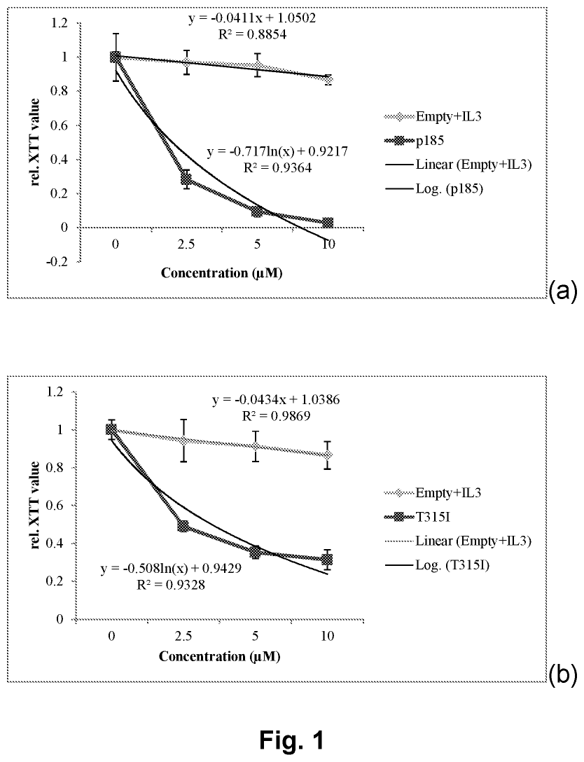

FIGS. 1A and 1B: Effects of Compound 16 on Ba/F3 cells expressing either p185-BCR-ABL or T315I-p185-BCR-ABL. Cells were exposed to increasing concentration of Compound 16 [empty vehicle (0.0 μM+IL3), 2.5, 5.0 and 10.0 μM]. FIG. 1A shows the inhibitory effect of concentration of Compound 16 [empty vehicle (0.0 μM+IL3), 2.5, 5.0 and 10.0 μM] on Ba/F3 cells expressing WT-p185-BCR-ABL, while FIG. 1B shows the inhibitory effect of concentration of Compound 16 [empty vehicle (0.0 μM+IL3), 2.5, 5.0 and 10.0 μM] on Ba/F3 cells expressing mutant T315I-p185-BCR-ABL. Cell viability (CV) was assessed using the XTT method as described in the experimental part. The means±SD of triplicates from one representative experiment out of three performed are given.

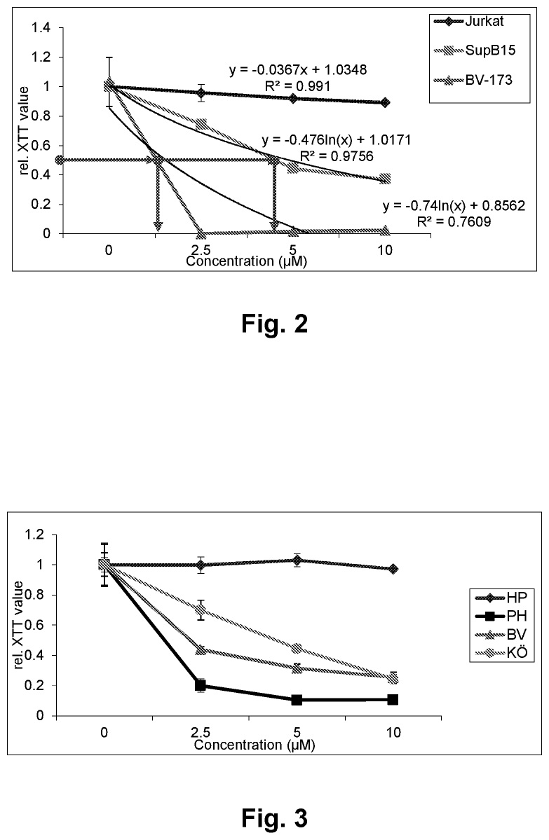

FIG. 2: Effect of Compound 16 on leukemia cell lines (Jurkat, Sup-B15 and BV173). Cell viablility (CV) was determined following incubation of the 1 m cells/ml of each cell line with increasing concentration of the Compound 16 [empty vehicle (0.0 μM+IL3), 2.5, 5.0 and 10.0 μM]. CV was measured by the XTT method. The means±SD of triplicates from one representative experiment out of three performed are given.

Jurkat (an immortalized T lymphocyte cell line that was originally obtained from the peripheral blood of a boy with T cell leukemia) taken as control were observed to be highly insensitive to Compound 16, while BV173 was most sensitive. When comparing the action against both Ph+ cell lines BV173 to SUP—B15.

The IC₅₀ of Compound 16 against BV173 was estimated to be approximately 2 μM, and against Sup-B15 was estimated to be approximately 4.5 μM. At 10 μM, around 69% of Sup-B15 cells were inhibited, while at 2.5 μM of Compound 16 around 100% of BV173 cells were wiped, indicating a differing sensitivity of the cells expressing p185-BCR-ABL compared others that express p210-BCR-ABL.

FIG. 3. Effects of Compound 16 on patient-derived long-term cultures (PD-LTCs): HP (Ph− ALL cultures used as negative controls, diamond), PH (Ph+ that is considered ABL Kinase Inhibitors (AKIs) sensitive culture, circle), BV173 (triangle) and KÖ (Ph+T315I-BCR-ABL that is considered ABL Kinase Inhibitors (AKIs) resistant culture, square). Cells were exposed to increasing concentration of 2.5, 5 and 10 μM of Compound 16. The negative control cell lines Ph− ALL HP was irresponsive to increasing concentration of Compound 16 (up to 10 μM). The three Ph+ cells (PH, BV173 and KÖ) were inhibited with variable degrees. All four cell lines were plated in duplicates at the indicated concentrations, and proliferation was assessed after three days by dye exclusion of viable cells [53]. Results represent the mean of 3 independent experiments+/−S.D. PH was most sensitive with IC₅₀ of [Compound 16]˜2.1 μM, while HP was irresponsive to Compound 16 at concentration as high as 10 μM. Though at lower concentrations BV173 exhibited a higher sensitivity towards Compound 16 (IC₅₀ 2.5 μM), at higher concentration of 10 μM both KÖ and BV have similar response. The sensitivity of Ph+ cells to Compound 16 underlines the involvement of BCR-ABL in the antiproliferative mechanism. Death induction of BV173 cells is well taken as an indication of the ability of such group of compounds to overcome resistance that is originated in non-mutational grounds. However, the ability overcome the resistant T315I-BCR-ABL mutant, as shown in the dose-dependent manner of KÖ (circles), might be indicative for a non-ATP competitive mechanism of action.

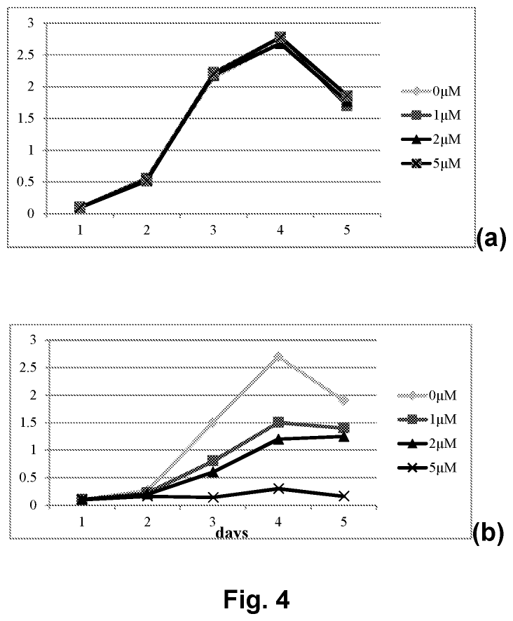

FIGS. 4A and 4B. Effect of Compound 16 on WT-Ba/F3 compared to p185-BCR-ABL-Ba/F3. Antiproliferative effect of Compound 16 on WT-Ba/F3 (FIG. 4A) compared to p185 expressing Ba/F3 cells (FIG. 4B). Cells were exposed to 1.0 and 2.0 and 5.0 μM of Compound 16. FIG. 4A shows the inhibition of wild type WT-Ba/F3 cell lines by Compound 16 (1.0 and 2.0 and 5.0 μM of Compound 16) compared to the control with not compound. FIG. 4B shows the inhibition of p185 expressing Ba/F3 cells (Compound 16) within the range of concentration (1.0 and 2.0 and 5.0 μM of Compound 16). Compound 16 inhibits the proliferation of p185-Ba/F3 (WT-Ba/F3-p185-BCR-ABL) cells in a dose dependent manner without affecting the proliferation of WT-Ba/F3. All cell lines were treated with the indicated concentrations of Compound 16 for total period of five days and proliferation was determined by trypan blue exclusion of viable cells. The means±SD of triplicates from one representative experiment out of three performed are given. The means±SD of triplicates from one representative experiment out of three performed are given. For factor-independent growth, the number of viable cells was determined at day 3 by trypan blue dye exclusion.

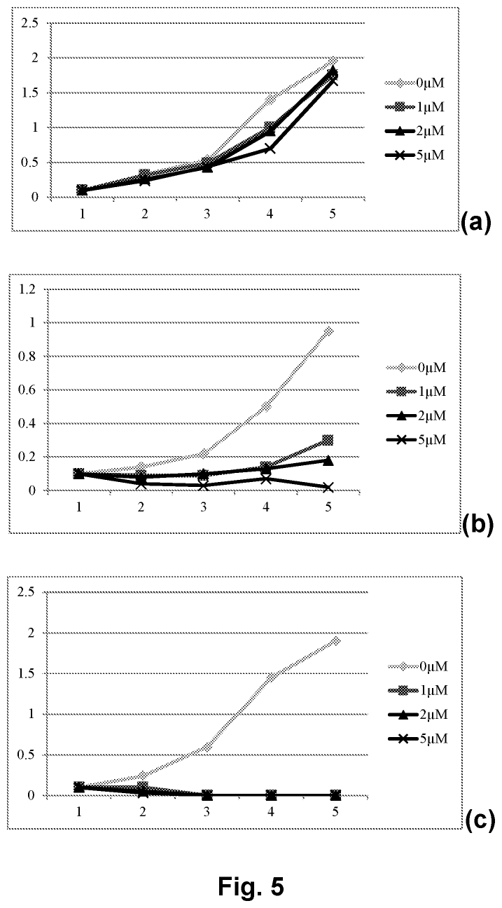

FIGS. 5A-C: Time-dependent inhibition of Ph+ Jurkat cells (taken as control, FIG. 5A) and Ph+PD-LTCs Sup-B15 (FIG. 5B) and BV173 (FIG. 5C) by Compound 16. Compound 16 was dissolved in DMSO and cells were incubated with the relevant concentration and cell viability (CV) was assessed using at the time indicated (in days), and IC₅₀ values were determined as described previously.

PH is selected as fully responsive to TKIs while PD-LTCs BV173 exhibits almost complete resistance to TKIs that is not attributable to mutations in the tyrosine kinase domain (TKD). Thus, the high responsiveness of BV173 to Compound 16 might indicate a way to overcome resistance that emerges as a result of factors other than KD mutations. A closer look at FIGS. 5A-C showed that Ph+BV173 cells are most sensitive towards Compound 16 relative to Jurkat cells. A result underlines the role of BCR-ABL isoform in induction of cellular response to treatment.

Effect of Compound 16 on Ph+ Jurkat (immortalized T lymphocytes derived from the peripheral blood of a child suffering from leukemia. Jurkat cells are often used to study acute T cell leukemia, T cell signaling, and the expression of various chemokine receptors in this experiment Jurkat cells are taken as control), Sup-B15 and BV173. Three Ph+ cells were used to assess the effect of Compound 16. Relevant concentration of the compound was added to seeded cells at day 1 (24 hrs) and cytotoxicity and proliferation were assessed at after 24, 48, 72, 96 and 120 h by XTT. The control was cells exposed to empty vehicle (0 μM, green line in each case), 1, 2 and 5 μM concentration of Compound 16 were administered to the three cell lines. It was noticed that BV173 is the most sensitive to Compound 16, i.e., 1 μM of Compound 16 was sufficient to block the proliferation of BV173 within 24 hours. Also, Compound 16 potently inhibited the proliferation of Sup-B15 cells while Jurkat cells were irresponsive to increased concentration of the compound. The means±SD of triplicates from one representative experiment out of three performed are given. It could be concluded that though Compound 16 inhibits Ph+ Sup-B15 and BV173 are responsive towards Compound 16 with the notion that p210-BCR-ABL leukemia cells at lymphoblastic blast crisis stage are quite more sensitive than p185-BCR-ABL ALL cells at corresponding inhibitor concentration.

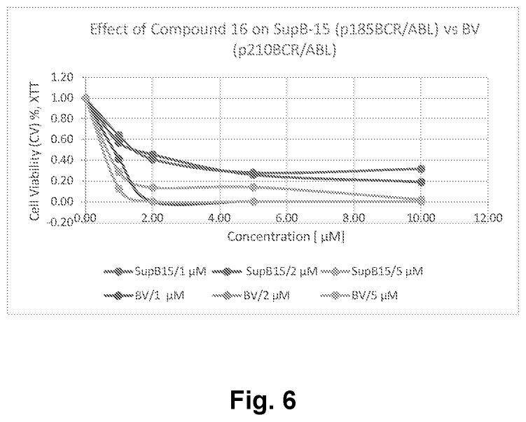

FIG. 6: Sensitivity of Sup-B15 (p185-BCR-ABL) compared BV173 cells (p210-BCR-ABL) towards increasing concentration of Compound 16. Ph+ cells were used to assess the effect of Compound 16. To assess the antiproliferative effect, cells were incubated with increasing concentration [0, 2, 4, 6, 8, and 10 μM] of Compound 16 and the viability was assessed using XTT. The means±SD of triplicates from one representative experiment out of three performed are given. Though both cells were responsive to increasing the dose., it was noticed, however, that BV173 cells were in general more sensitive to Compound 16 than Sup-B15 cell. BV173 propagation was fully blocked by 1 μM of Compound 16 following 24 hours exposure. Such results might indicate the sensitivity of p210-BCR-ABL expressing cells to the compounds in hand. BV173 cells in all cases a plateau in antiproliferative effect of the compound was noticed.

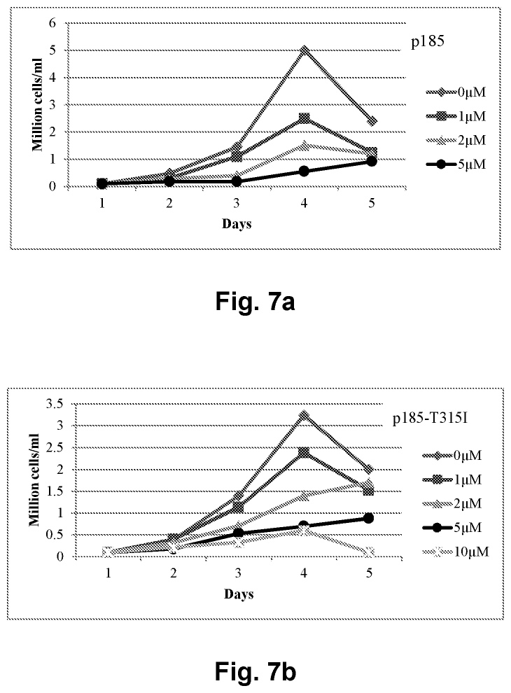

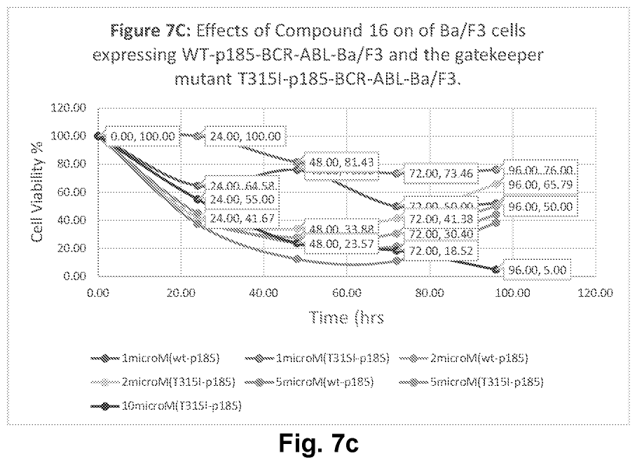

FIGS. 7A-C: Effects of Compound 16 on of Ba/F3 cells expressing WT-p185-BCR-ABL (FIG. 7A) and the mutant gatekeeper T315I-p185-BCR-ABL (FIG. 7B) by Compound 16. Cells were exposed to increasing concentrations of Compound 16. Control cells were exposed to empty vehicle (0 μM, diamond line in both cases), and 1, 2, 5 and 10 μM of Compound 16. Both cells were responsive to the compound to similar extent within the range (1-5 μM). However, at 10 μM Ba/F3 cells expressing T315I-p185-BCR-ABL were eliminated after 5 days exposure. The results indicated the ability of Compound 16 to overcome the resistance induced by the gatekeeper mutation in Ba/F3 cells. FIG. 7c is a plot of time-dependent inhibitory effects of Compound 16 on either WT-p185-BCR-ABL-Ba/F3 or the gatekeeper mutant T315I-p185-BCR-ABL-Ba/F3 expressing cells. Cells were exposed to increasing concentrations of Compound 16 [1, 2, 5 and 10 μM] and readings were taken at 12, 24, 48, 72, and 96 hours. Within the first 24 hours a difference in the response was noticed at 1 μM of Compound 16. While 35% of WT-p185-BCR-ABL-Ba/F3 cells were inhibited the mutated T315I-p185-BCR-ABL-Ba/F3 cells were not affected. When using 5 μM of Compound 16, the maximum inhibition was detected at −60 hours post the application, which is also the time-point where the wider difference against WT-p185—compared to T315I-p185-BCR-ABL expressing Ba/F3 cells. When applying 10 μM concentration, a complete inhibition of the mutant T315I-p185-BCR-ABL was achieved after ˜96 hours (5 days). This indicates the ability of Compound 16 to overcome the resistance induced by the gatekeeper mutation in Ba/F3 cells and eliminate T315I-p185-BCR-ABL expressing Ba/F3 after 5 days exposure.

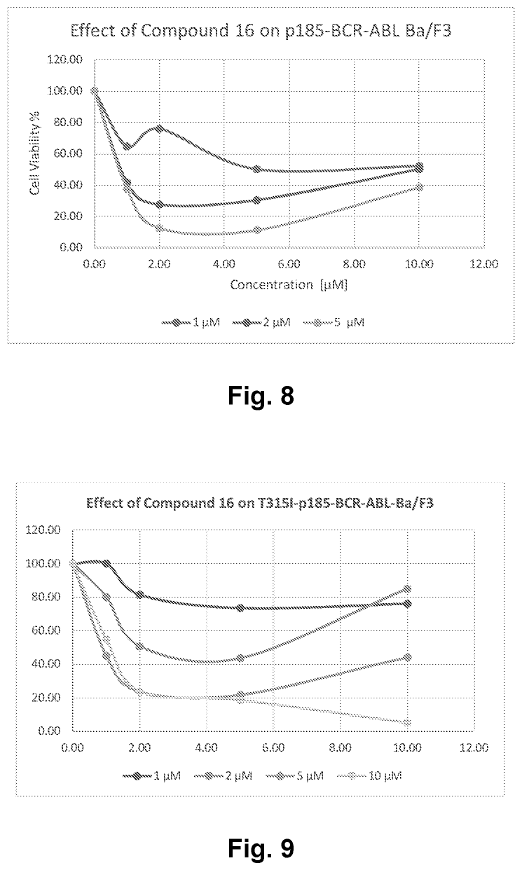

FIG. 8: Inhibition of p185-BCR-ABL Ba/F3 by Compound 16. At higher concentrations of 5 μM (gray) cell proliferation was affected significantly (˜90%) compared to lower concentrations 1 (blue) and 2 μM (orange). Interestingly the growth recovery was noticed at 2 and 5 μM.

FIG. 9: Inhibition of T315I-p185-BCR-ABL-Ba/F3 cells by Compound 16. For example, when both cells were exposed to 2 μM, at T=48 hr. 27.59% of p185-Ba/F3 cells were viable compared to 50.71% of T315I-p185-BCR-ABL-Ba/F3 cells i.e., T315I mutant of Ba/F3 cells confers 1.84-fold resistance. At higher concentrations of 5 (gray) and 10 μM (yellow) cell proliferation was affected significantly (˜80%) compared to lower concentrations 1 (blue) and 2 μM (orange). Interestingly the growth recovery was noticed at 2 and 5 μM.

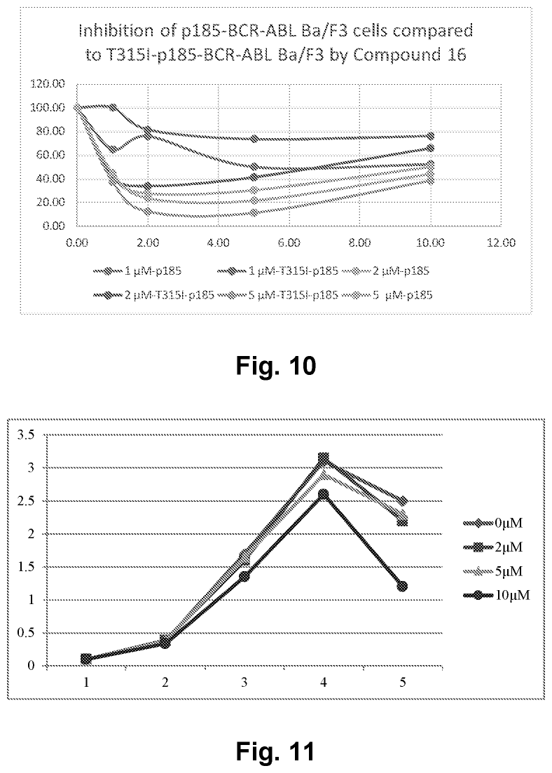

FIG. 10: Comparison of cell viability (CV) calculated as the ratio between the number of living cells following exposure to applied concentration and the number of control cells (exposed to empty vehicle) at selected time point. At (5 μM, 48 hr.) CV was 12.41% and 23.57% for p185-Ba/F3 and T315I-p185-Ba/F3 cells respectively. Interestingly both cell lines exhibited comparable sensitivity towards Compound 16 at lower concentrations. For example, (1 μM, 48 hr.) CV of was 75.86% and 81.43% (i.e., RF=1.07) for p185-Ba/F3 and T315I-p185 Ba/F3 cells respectively. The effectivity of Compound 16 was more profound against p185-BCR-ABL When than T315I-p185-BCR-ABL Ba/F3 cells (FIGS. 9 and 10 respectively).

FIG. 11: Antiproliferative effect of Compound 16 on PINCO transfected Ba/F3 cells. Ba/F3 cells expressing BCR-ABL constructs exhibit resistance to the compound. Ba/F3 cells are transfected with PINCO, when they are transduced with the empty vector as a control that factor independency is not due to the retroviral vector. The XTT assay was carried out on Ba/F3 cells expressing p185-BCR-ABL upon exposure to 2.0, 5.0 and 10.0 μM of Compound 16. Proliferation status was determined by the metabolic activity of cells given by the reduction rate of XTT to formazan. The means+/−SD of triplicates from one representative experiment out of three performed are given. No effect was observed on the proliferation of PINCO transfected cells upon treatment with 2.0, 5.0 and 10.0 μM of Compound 16.

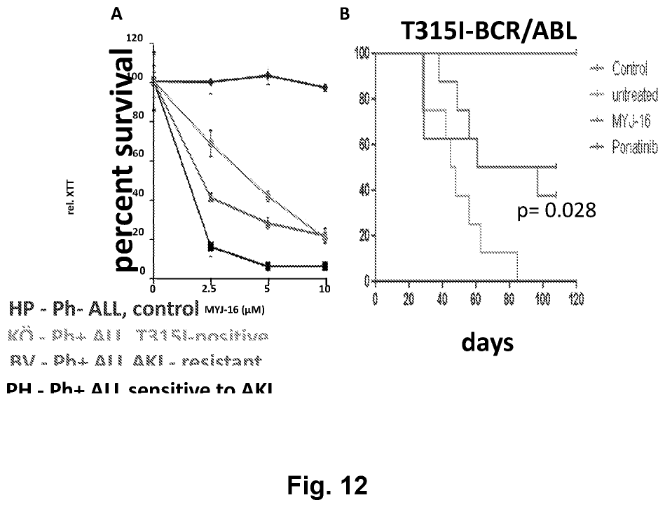

FIGS. 12A and 12B: Tumor Reduction in animal models [56]. T315I-positive Ph+ ALL xenograft model. T315I-BCR-ABL positive PD-LTC (KÖ) cells (4×10⁶) were inoculated via tail vein into sublethally irradiated (2.5 Gy) NOD.Cg-Prkdcscid Il2rgtm1Wjl/SzJ (NSG) mice. These mice were bred at the animal facility of the Georg-Speyer Haus, Frankfurt, Germany, under specific pathogen-free conditions. Mice were killed at the first appearance of morbidity.

### Example 2: Inhibition Assays of Compound 30

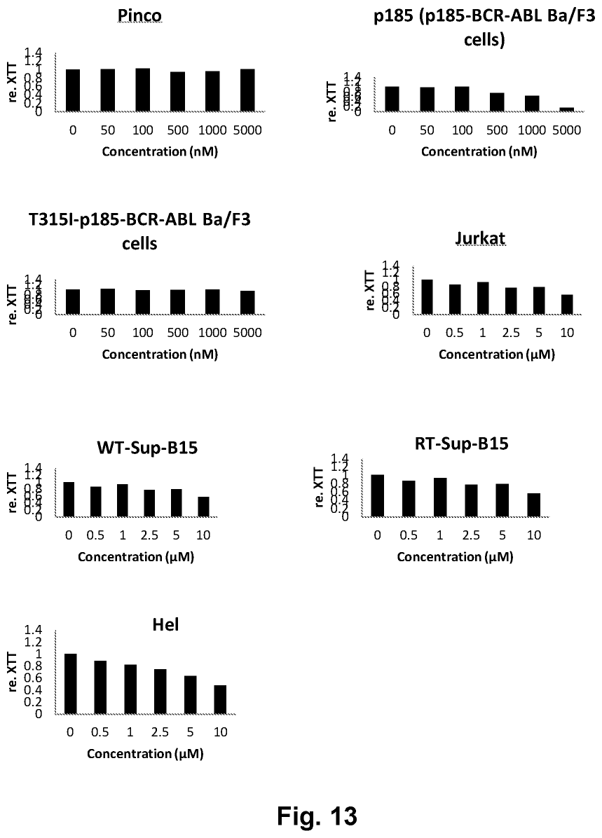

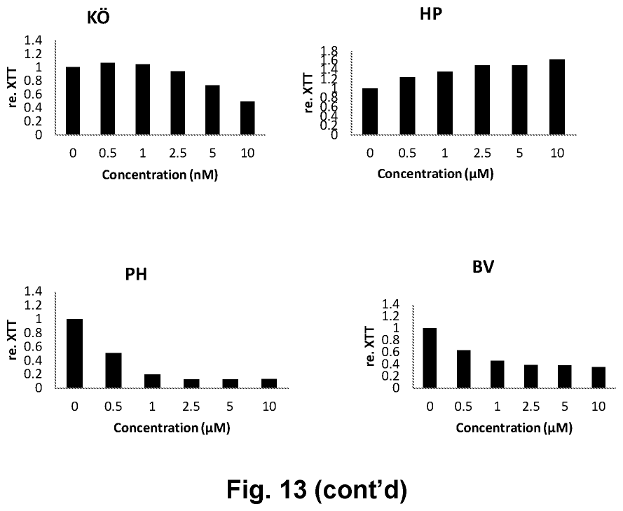

FIG. 13: Effect of Compound 30 on PINCO, p185 (WT-p185-BCR-ABL, Ph+ CML), T315I-p185 (T315I-p185-BCR-ABL, Ph+ CML), Jurkat, WT-Sup-B15, RT-Sup-B15, BV, HEL, HP, PH, and KÖ.

In the case of Compound 30, the compound was dissolved in DMSO and the cells were incubated with increased concentration starting at 0.5, 1.0, 2.5, 5.0 up to 10.0 μM. Cell viability (CV) was assessed using the XTT method and compared to vehicle (0 μM of the compound in each case). PH and BV showed increased sensitivity to Compound 30 compared to other cells including Ba/F3(PINCO), p185-Ba/F3, T315I-p185-Ba/F3, Jurkat, WT-Sup-B15, RT-Sup-B15, HEL, HP, and KÖ. Interestingly, both PH and BV are PD-LTC from a Ph+ that are considered p210-BCR-ABL dependent CML.

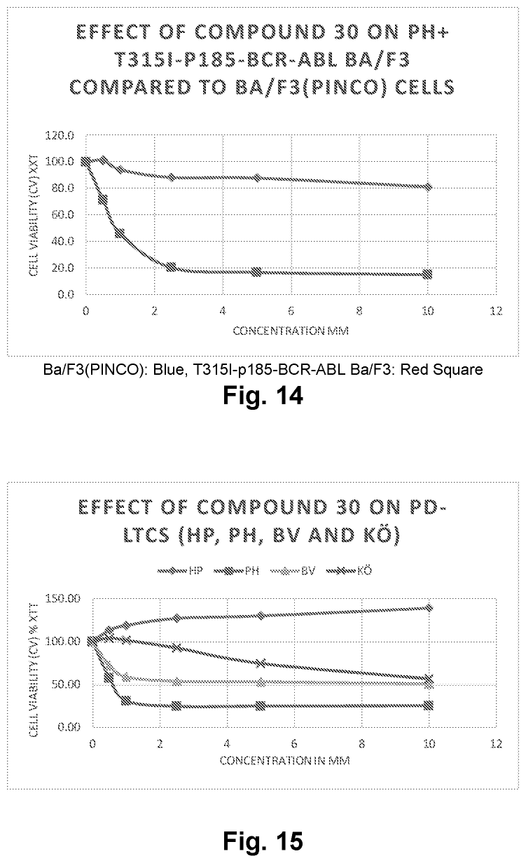

FIG. 14: Comparison of inhibitory action of Compound 30 against Ph+T315I-p185-BCR-ABL CML cells compared to Ba/F3(PINCO). In T315I-BCR-ABL CML cells the compound exerts a concentration dependent inhibition of cell propagation between 0.5, 1.0, 2.5, and 4.0 μM. Afterwards, a plateau in C/R (CV˜16%) was observed for the concentration 4 and 10.0 μM.

FIG. 15: Concentration-Response of Compound 30 against PD-LTCs HP, PH, BV and KÖ.

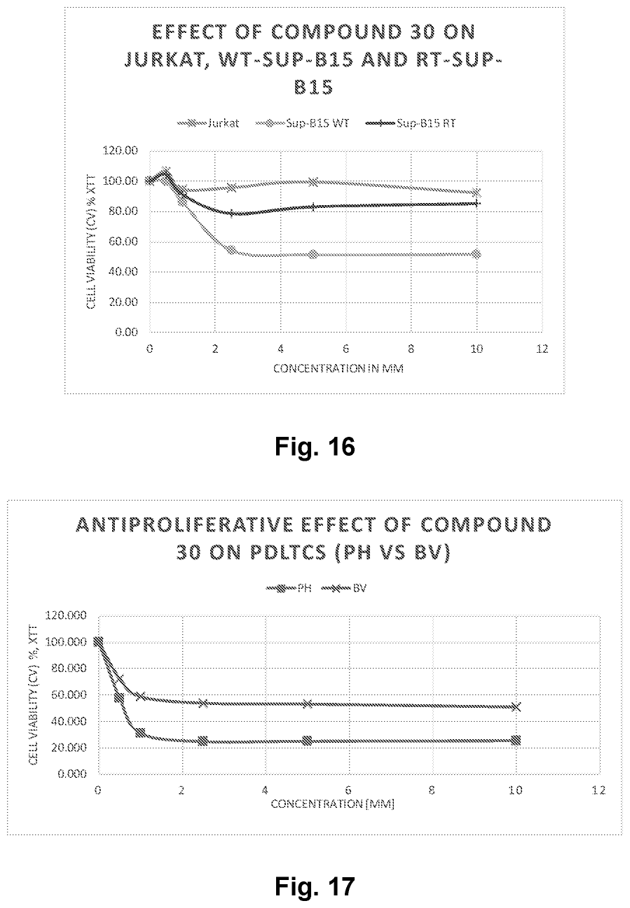

FIG. 16: Concentration-Response of Compound 30 against Jurkat, WT-Sup-B15 and RT-Sup-B15. Comparison of inhibitory action of Compound 30 against Jurkat, WT-Sup-B15, RT-Sup-B15. Cells in each case were exposed to increasing concentration of Compound 30 [0.5, 1.0, 2.5, 5.0, and 10.0 μM]. Jurkat, and RT-Sup-B15 were irresponsive towards Compound 30 within the indicated concentrations. The sensitive cells WT-Sup-B15 was responsive to increasing the concentration of the compound at concentrations lower than 2.5 μM and reached a plateau in C/R when concentration was increased (between 2.5 and 10.0 μM). It could be designated that WT-Sup-B15 cells were twice as sensitive to the compound as WT-Sup-B15.

FIG. 17: The concentration-response (C/R) as measured by the cell viability (CV) following exposure to increasing dose of Compound 30. The order of response of PD-LTCs was PH>BV. PH is Ph+p185-BCR-ABL ALL that is sensitive to ABL Kinase Inhibitors (AKIs), while BV is Ph+ CML in myeloid blast crisis p210-BCR-ABL indicates that Compound 30 exhibits potent inhibitory effect against the shorter p185-BCR-ABL-dependent cultures. The concentration-response (C/R) of the two sensitive PH and BV plateaus within the range of concentration 2.5 and 10 μM indicating inefficacious action of high doses (>2.5 μM).

The inhibition of sensitive PD-LTCs cells (PH, BV) by Compound 30. The compound was effective at lower concentration (between 1 μM and 2.5 μM).

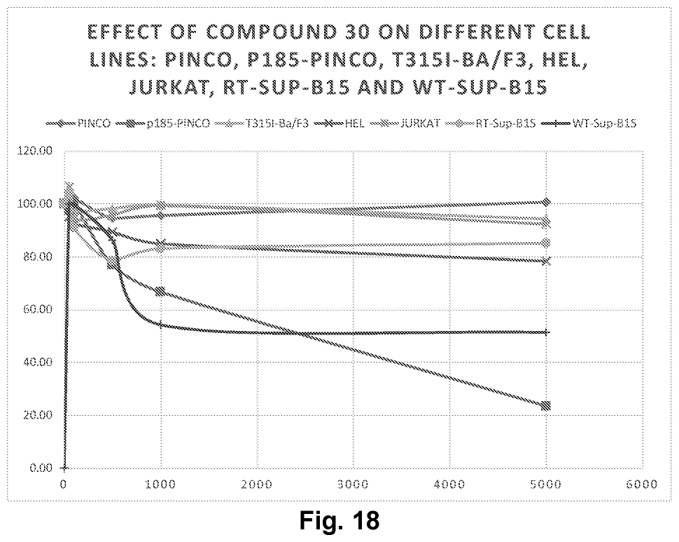

FIG. 18: Antiproliferative effect of Compound 30 on the following models: a) patient-derived long-term cell culture system (PD-LTCs): HP, BV and PH and b) on transduced Ba/F3 comprising empty vector (PINCO), p185-BCR-ABL-Ba/F3, T315I-p185-BCR-ABL-Ba/F3 and c) cell lines comprising RT-Sup-B15, BV173, Ph− V617FJAK2 HEL and Jurkat. The activity of the compound was plotted as cell viability (CV) of the exposed cell at the y-axis at the correspondent concentration in nanomolar (nM) on the x-axis using the XTT method.

Transduced Ba/F3, T315I-BCR-ABL-Ba/F3, RT-Sup-B15 and Ph− HEL, cells were weakly responsive to increasing concentration of the compound. Of notice was the increased sensitivity of plasmid transfected p185-PINCO and WT-Sup-B15. While the response of WT-Sup-B15 cells was plateaued between 1000 and 5000 nM (FIG. 18, plus sign), the inhibitory effect of the Compound 30 on p185-PINCO cells was profoundly detected at higher concentration (FIG. 18, square). Ph− V617FJAK2 HEL and KÖ (T315I-BCR-ABL positive) is negligible at low concentrations (<1 μM). A weak inhibition is noticed at concentration that ranges between 2.5-10 μM.

### Example 3: Inhibition Assays of Compound 31

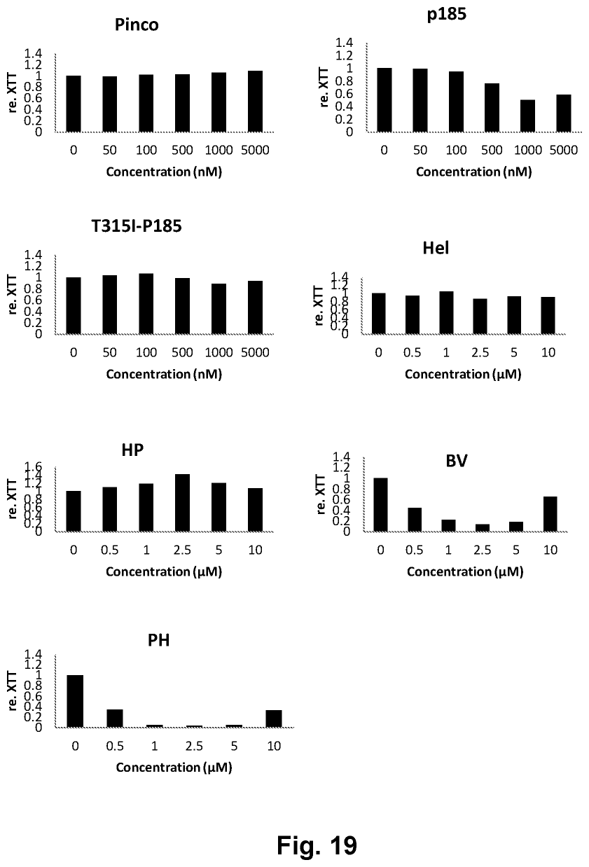

FIG. 19: Antiproliferative effect of Compound 31 on the following models: a) patient-derived long-term cell culture system (PD-LTCs): HP, BV, PH and KÖ, and b) transduced Ba/F3 comprising empty vector (PINCO), p185-BCR-ABL-Ba/F3, T315I-p185-BCR-ABL-Ba/F3 and c) cell lines comprising RT-Sup-B15, BV173, Ph− V617FJAK2 HEL and Jurkat. The activity of the compound was plotted as cell viability (CV) of the exposed cell at the y-axis at the correspondent concentration in nanomolar (nM) on the x-axis using the XTT method. The inhibitory effect of Compound 31 against p185-Ba/F3, T315I-p185-Ba/F3, Ph− HEL and the resistant HP was negligible at increasing concentration up to 10 μM. On the other hand, proliferation of BV and PH PD-LTCs was significantly inhibited in a dose-response manner with higher response at PH cultures than BV.

In the case of Compound 31, the compound was dissolved in DMSO and the cells were incubated with increased concentration starting at 0.5, 1.0, 2.5, 5.0 and 10.0 μM. Cell viability (CV) was assessed using the XTT method and compared to vehicle (0 μM of the compound in each case). PD-LTCs PH and BV showed increased sensitivity to Compound 31 compared to transduced cells including Ba/F3 (PINCO), p185-BCR-ABL-Ba/F3, T315I-p185-BCR-ABL-Ba/F or to HEL, BV, HP, and KÖ. Interestingly, both PH and BV are PD-LTCs from a Ph+ patients and considered p210-BCR-ABL-dependent ALL.

### Example 4: Inhibition Assays of Compound 32

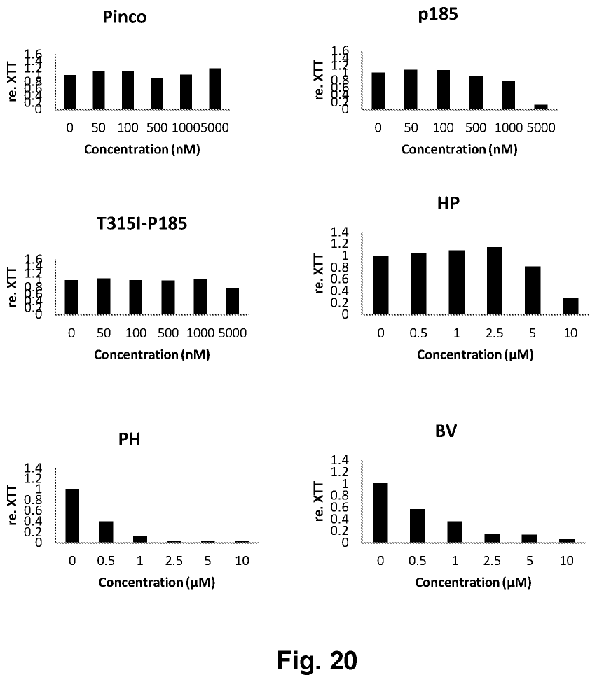

FIG. 20: Antiproliferative effect of Compound 32 on the following models: a) patient-derived long-term cell culture system (PD-LTCs): HP, BV, PH and KÖ, and b) transduced Ba/F3 comprising empty vector (PINCO), p185-BCR-ABL-Ba/F3, T315I-p185-BCR-ABL-Ba/F3 and c) cell lines comprising RT-Sup-B15, BV173, Ph− V617FJAK2 HEL and Jurkat. The activity of the compound was plotted as cell viability (CV) of the exposed cell at the y-axis at the corresponding concentration in nanomolar (nM) on the x-axis using the XTT method.

### Example 5: Inhibition Assays of Compound 33

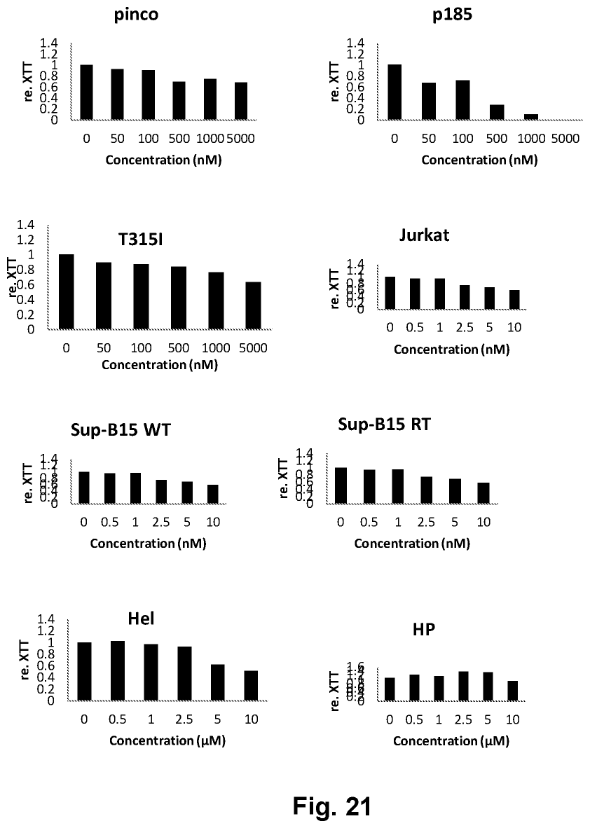

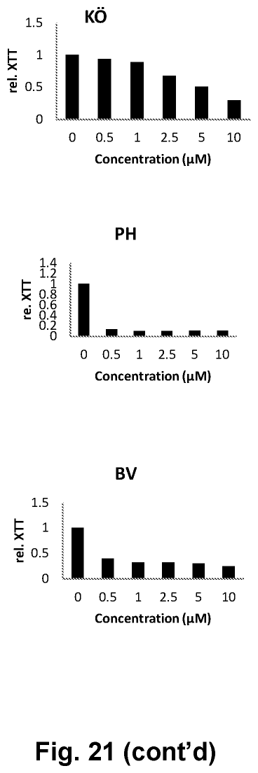

FIG. 21: Antiproliferative effect of Compound 33 on the following models: a) patient-derived long-term cell culture system (PD-LTCs): HP, BV, PH and KÖ, and b) transduced Ba/F3 comprising empty vector (PINCO), p185-BCR-ABL-Ba/F3, T315I-p185-BCR-ABL-Ba/F3 and c) cell lines comprising RT-Sup-B15, BV173, Ph− V617FJAK2 HEL and Jurkat. The activity of the compound was plotted as cell viability (CV) of the exposed cell at the y-axis at the corresponding concentration in nanomolar (nM) on the x-axis using XTT method.

### Example 6: Inhibition Assays of Compound 34

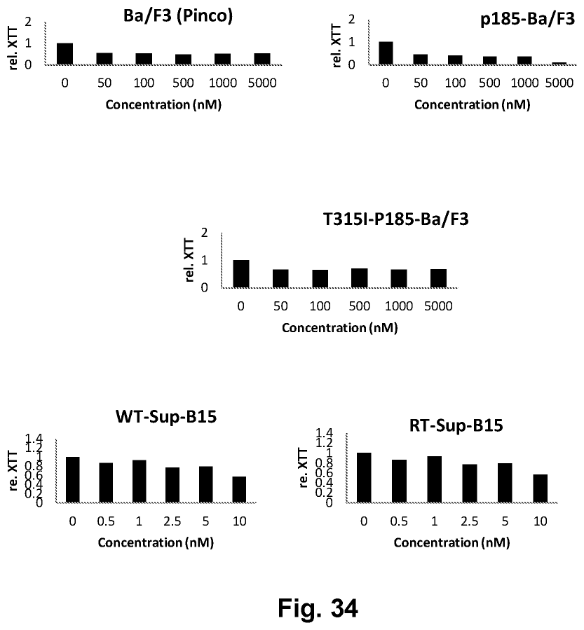

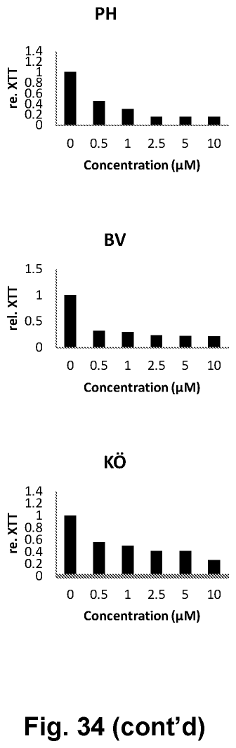

FIG. 34: Concentration-Response (C/R) of Compound 34 against Ba/F3(PINCO) p185-BCR-ABL-Ba/F3, T315I-p185-BCR-ABL-Ba/F, WT-Sup-B15, RT-Sup-B15, HEL and patient-derived long-term cell culture system (PD-LTCs): BV, HP, KÖ and PH. Antiproliferative effect of Compound 34 was assessed by exposing cell lines and patient-derived long-term cell culture system (PD-LTCs) to increasing concentration [0.5 μM-10.0 μM] of the compound in each case. Cell viability (CV) was assessed using the XTT method. Cell viability ratio (CV %) was calculated by averaging the triplicate, normalizing to inhibition of compound-free vehicle and calculating the ratio.

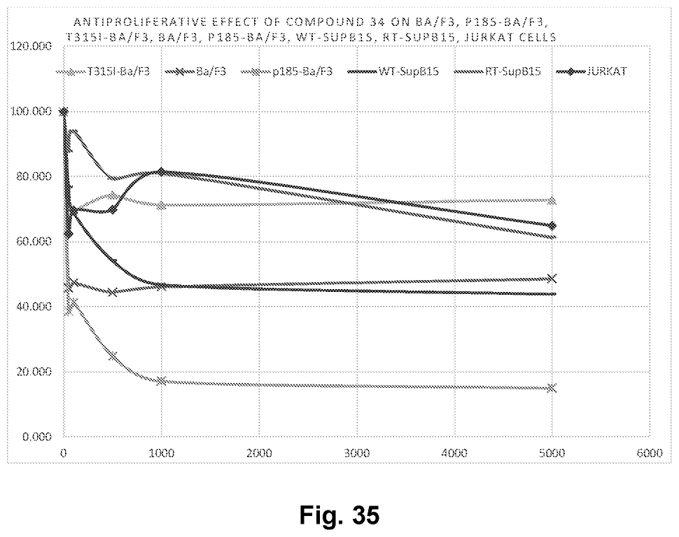

FIG. 35: Antiproliferative Effect of Compound 34 the following models: a) transduced Ba/F3 comprising empty vector (PINCO), p185-BCR-ABL-Ba/F3, T315I-p185-BCR-ABL-Ba/F3 and b) cell lines comprising WT-SupB15, RT-Sup-B15, and Jurkat. Compound 34 potently inhibits Ba/F3 cells. The most sensitive cell line to the compound was WT-p185-Ba/F3 while T315I-p185-Ba/F3 was least responsive whereas transduced Ba/F3 (PINCO) and WT-SubP15 were intermediately responsive. The response was plateaued at concentrations higher than 1000 nM.

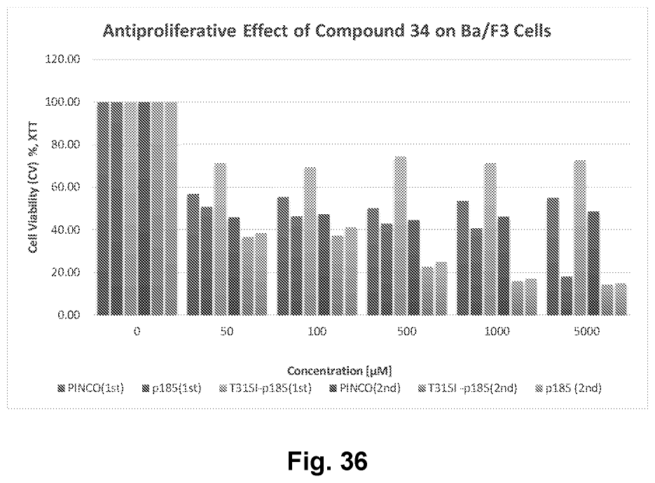

FIG. 36: Antiproliferative Effect of Compound 34 on leukemia cell lines: Ba/F3(PINCO), p185-Ba/F3, T315I-p185-Ba/F3.

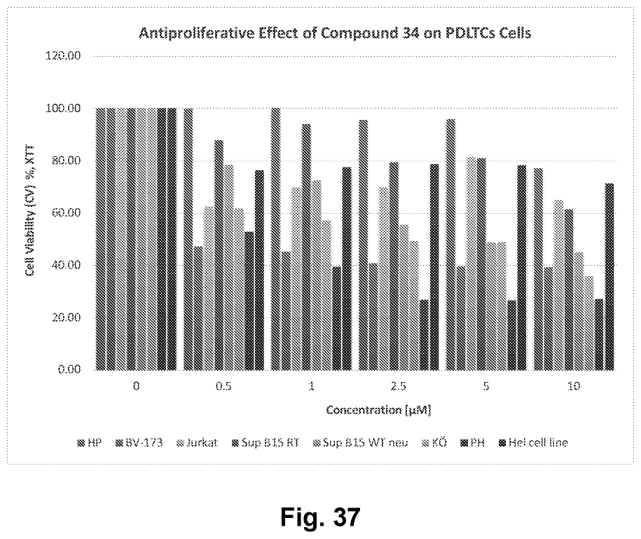

FIG. 37: Antiproliferative Effect of Compound 34 on the following models: a) transduced Ba/F3 comprising empty vector (PINCO), p185-BCR-ABL-Ba/F3, T315I-p185-BCR-ABL-Ba/F3 and b) cell lines comprising RT-Sup-B15, Ph− V617FJAK2 HEL and Jurkat. The cell line p185-Ba/F3 (square) was most sensitive to increasing the concentration of the compound while WT-SupB15 (circle) and Ba/F3 (diamond) were the next in response that got plateaued and concentration higher than 1 μM. Transfection of p185-BCR-ABL to Ba/F3 cells potentiate the compound indication the involvement of BCR-ABL in its antiproliferative action. Other cells tested in this study (T315I—Ba/F3, Jurkat, WT-SupB15 and RT-SupB15) were irresponsive.

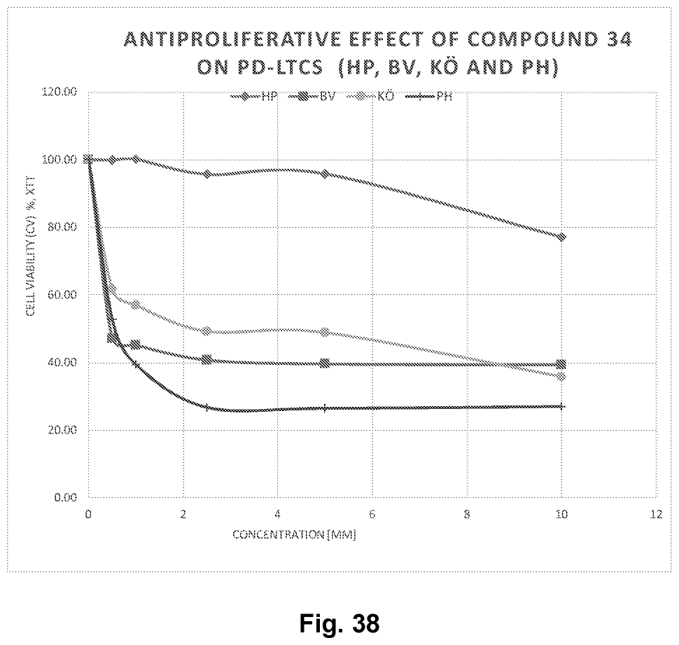

FIG. 38: Concentration-Response (C/R) of Compound 34 on PD-LTCs (HP, BV, KO and PH). While HP cells were highly resistant, PH cells were most responsive to increasing concentration of Compound 34. Interestingly, at concentration higher than 2.5 μM the response of sensitive cells PH and BV appeared to get plateaued with little response by KO culture at higher concentration of the compound.

TABLE 1
Inhibitory concentration 50%
(IC₅₀) of Compound 34 against PD-LTC.

| PD-LTC | IC₅₀ (μM) |
|---|---|
| PH | 0.13 |
| BV | 0.45 |
| KÖ | 2.37 |
| WT-Sup-B15 | 28.56 |

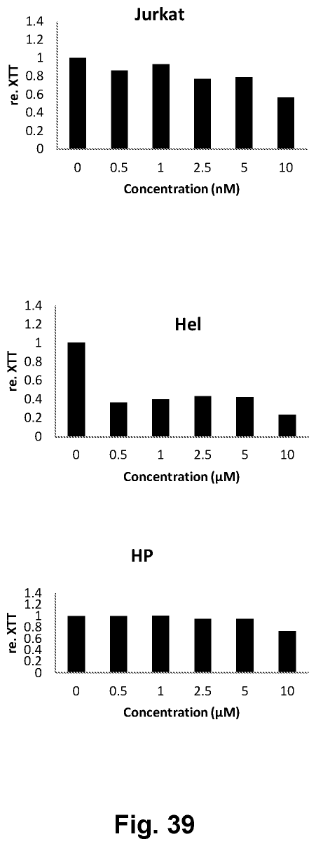

FIG. 39: Concentration-Response (C/R) of Compound 34 against Ph− cells: Jurkat, HEL and HP. Cells in each case were exposed to increasing concentration of Compound 34 [0.5, 1.0, 2.5, 5.0, and 10.0 μM]. Cell viability (CV) was assessed using the XTT method. Cell viability ratio (CV %) was calculated by averaging the triplicate, normalizing to inhibition of compound-free vehicle and calculating the ratio.

Discussion of Activity of Compound 34: Though the compound exerts moderate growth inhibition against KÖ culture, its potency against HP, Jurkat, and RT-Sup-B15 was negligible even at concertation higher than 5 μM. The lack of response of those cells can be due to the absence of biological target, prevalence of mutated isoforms, or to other resistance mechanisms. The response of WT-Sup-B15 cells and of BV and PH cultures is varied. variably respond to increasing the concentration of the compound at concentrations lower than 2.5 μM and reached a plateau in C/R when concentration was increased (between 2.5 and 10.0 μM). It could be indicated that PH cells were twice as sensitive to the compound as WT-Sup-B15 or BV culture which might prove that inhibitory action of Compound 34 is correlated with the level of dependency cell viability on BCR-ABL.

### Example 6: Inhibition Assays of Compound 35

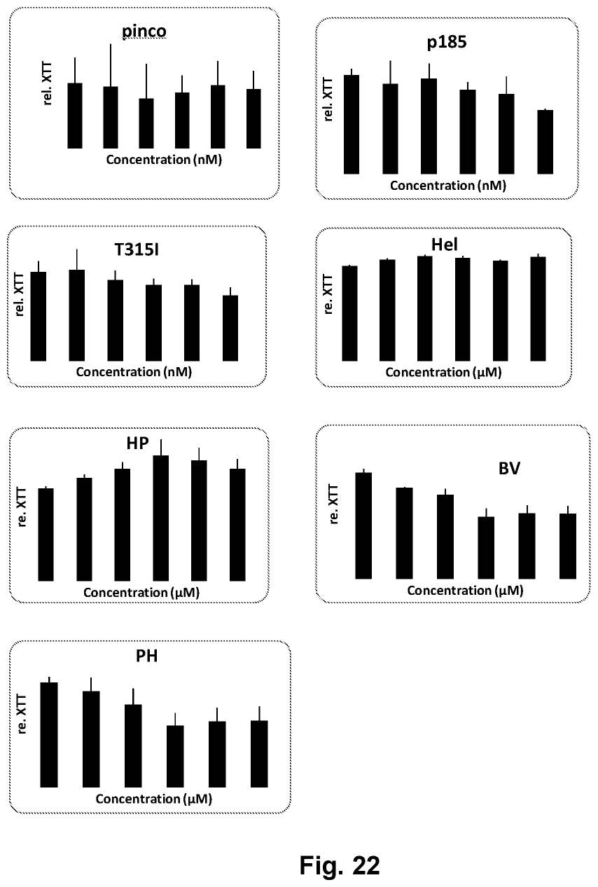

FIG. 22: Effect of Compound 35 on the following models: a) patient-derived long-term cell culture system (PD-LTCs): HP, BV and PH and b) on transduced Ba/F3 comprising empty vector (PINCO), p185-BCR-ABL-Ba/F3, T315I-p185-BCR-ABL-Ba/F3 and c) cell lines comprising RT-Sup-B15, BV173, Ph− V617FJAK2 HEL and Jurkat. The activity of the compound was plotted as cell viability (CV) of the exposed cell at the y-axis at the correspondent concentration in nanomolar (nM) on the x-axis using the XTT method.

### Example 7: Inhibition Assays of Compound 36

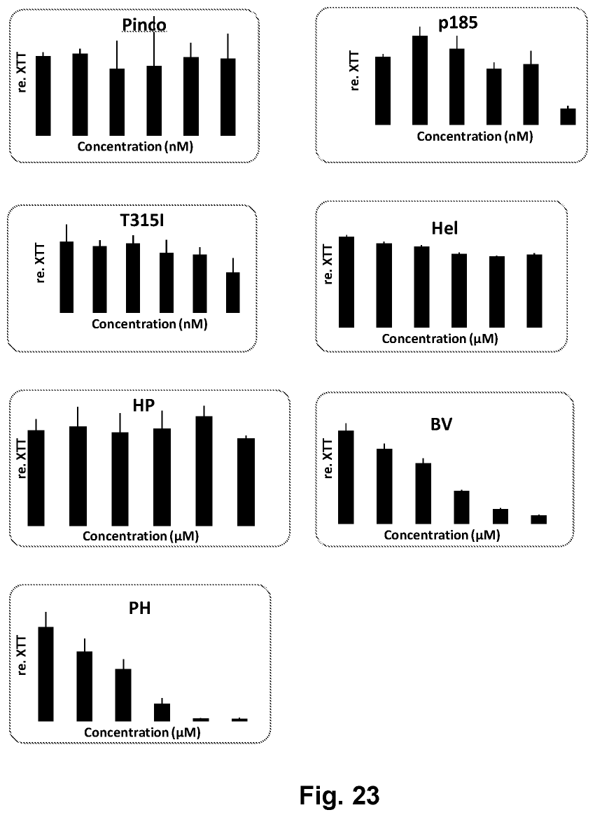

FIG. 23: Effect of Compound 36 on patient-derived long-term cell culture system (PD-LTCs): HP, BV and PH and on cell lines Ba/F3(PINCO), p185-BCR-ABL-Ba/F3, T315I-p185-BCR-ABL-Ba/F3 and Ph− HEL cells. The activity of the compound was plotted as cell viability (CV) of the exposed cell at the y-axis at the correspondent concentration in nanomolar (nM) on the x-axis using the XTT method.

### Example 8: Inhibition Assays of Compound 37

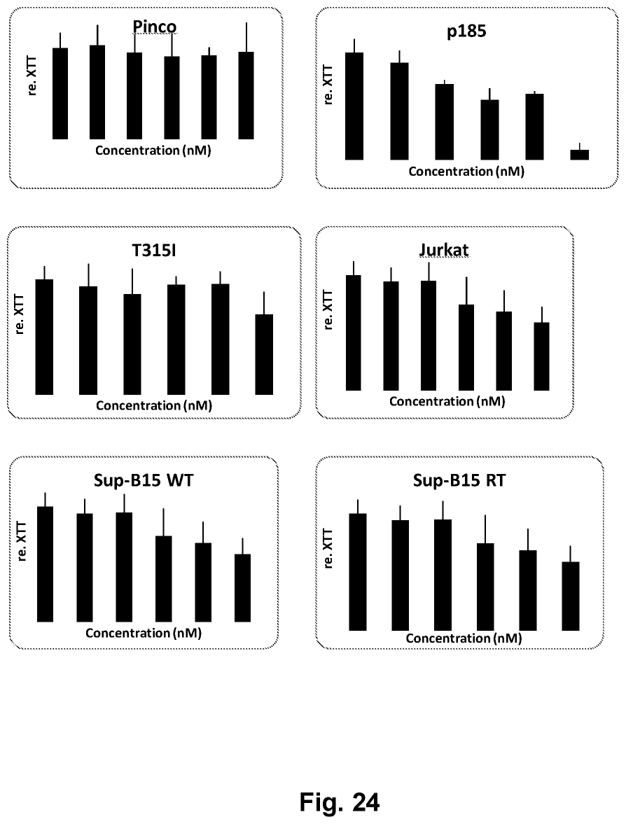

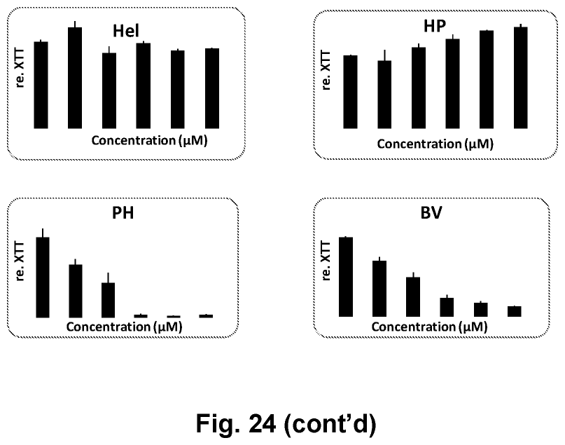

FIG. 24: Effect of Compound 37 on patient-derived long-term cell culture system (PD-LTCs): HP, BV and PH and on cell lines Ba/F3(PINCO), p185-BCR-ABL-Ba/F3(PINCO), T315I-p185-BCR-ABL-Ba/F3, WT-Sup-B15, RT-Sup-B15, KÖ, and Jurkat. and HEL. The activity of the compound was plotted as cell viability (CV) of the exposed cell at the y-axis at the correspondent concentration in nanomolar (nM) on the x-axis using the XTT method.

### Example 9: Inhibition Assays of Compound 38

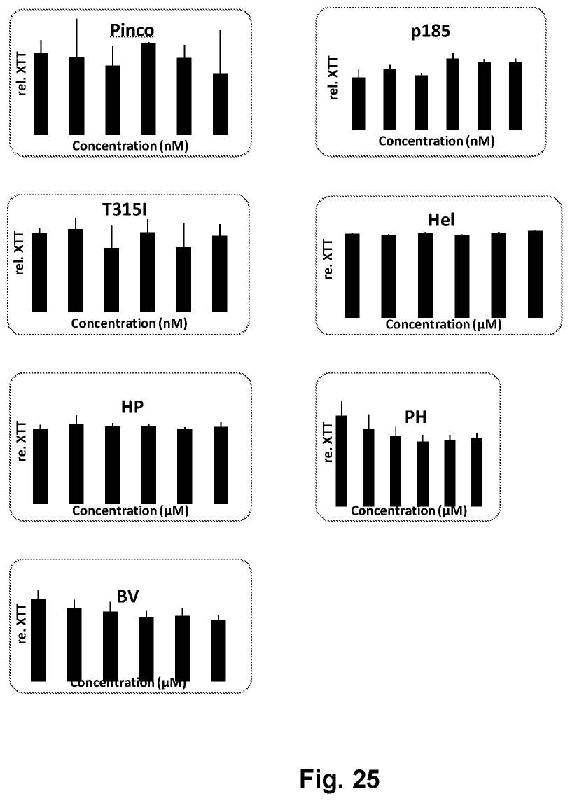

FIG. 25: Effect of Compound 38 on patient-derived long-term cell culture system (PD-LTCs): HP, BV and PH and on cell lines Ba/F3(PINCO), p185-BCR-ABL-Ba/F3(PINCO), T315I-p185-BCR-ABL-Ba/F3(PINCO) WT-Sup-B15, and HEL. The activity of the compound was plotted as cell viability (CV) of the exposed cell at the y-axis at the correspondent concentration in nanomolar (nM) on the x-axis using the XTT method.

### Example 10: Inhibition Assays of Compound 39

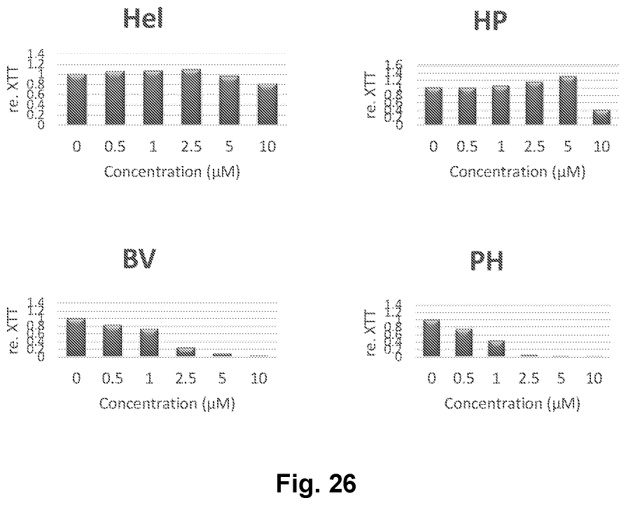

FIG. 26: Effect of Compound 39 on patient-derived long-term cell culture system (PD-LTCs): BV HP, and PH and Ph− cell line HEL. The activity of the compound was plotted as cell viability (CV) of the exposed cell at the y-axis at the correspondent concentration in nanomolar (nM) on the x-axis using the XTT method.

### Example 11: Inhibition Assays of Compound 40

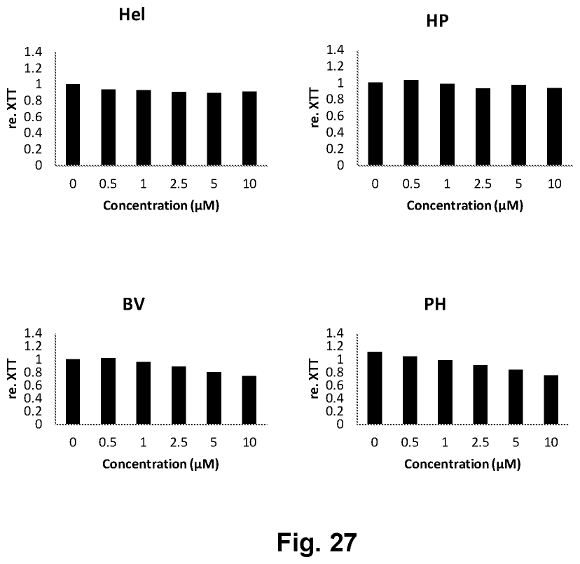

FIG. 27: Effect of Compound 40 on patient-derived long-term cell culture system (PD-LTCs): BV, HP and PH and Ph− cell line HEL. The activity of the compound was plotted as cell viability (CV) of the exposed cell at the y-axis at the correspondent concentration in nanomolar (nM) on the x-axis using the XTT method.

### Example 12: Inhibition Assays of Compound 41

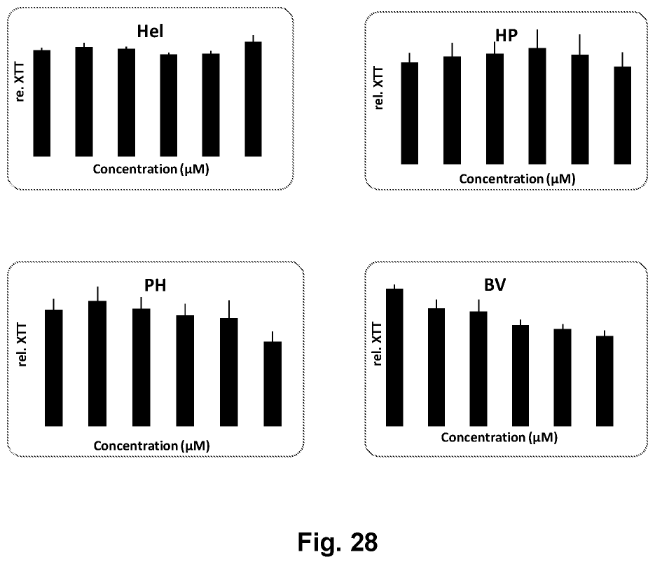

FIG. 28: Effect of Compound 41 on patient-derived long-term cell culture system (PD-LTCs): HEL, BV, HP and PH. The activity of the compound was plotted as cell viability (CV) of the exposed cell at the y-axis at the correspondent concentration in nanomolar (nM) on the x-axis using the XTT method.

### Example 13: Structure Activity Relationship of Compounds 31, 32 and 33

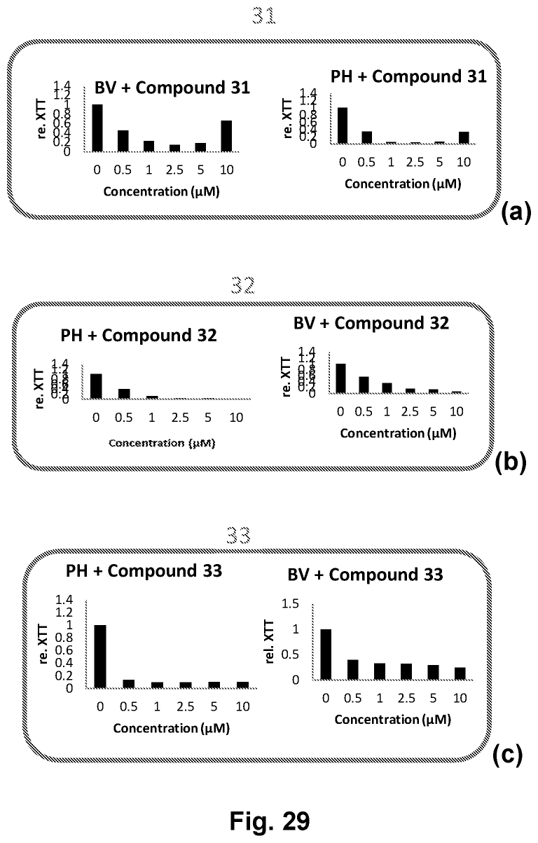

FIGS. 29A-C: Structure Activity Relationship (SAR) of Compounds 31, 32 and 33. Antiproliferative effect of Compounds 31 (FIG. 29A), 32 (FIG. 29B) and 33 (FIG. 29C) against (PD-LTCs): HP and BV cultures. Cell viability (CV) was assessed using the XTT method.

Compounds 31, 32 and 33 exhibited enhanced activity against PH and BV cells. Compounds 32 and 33 showed a good inhibitory effect when PH cells (Ph+ ALL cells known to be sensitive to AbN kinase inhibitors (AKIs)) were incubated with at the increased concentrations [0.5, 1.0, 2.5, 5.0, 10.0 μM]. Such effect of both compounds was milder when BV cells (Ph+ ALL AKI-resistant, p210-BCR-ABL CML in myeloid blast crisis cells) were incubated with similar concentrations. At concentration >5.0 μM of Compound 31, an induction of cell proliferation was noticed in both cells PH and BV. This effect was not noticed for Compounds 31, 32 and 33.

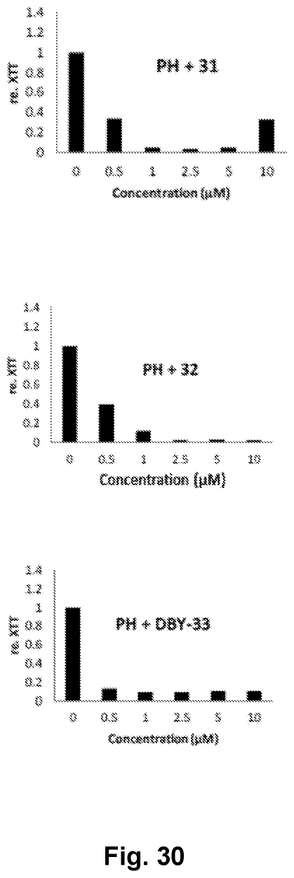

FIG. 30: Structure Activity Relationship of Compounds 31, 32 and 33 against PH cells. The compounds were assessed against patient-derived long-term cell culture system (PD-LTC) PH. The activity of the compound was plotted as cell viability (CV) of the exposed cell at the y-axis at the correspondent concentration in nanomolar (nM) on the x-axis using the XTT method. Compound 33 showed a sharp concentration response effect on PH cells compared to Compounds 31 and 32 at lower concentration (<0.5 μM). The effect was almost identical at concentrations of 1 and 5 μM for Compounds 31, 32 and 33. Compound 33 showed a sharp concentration response effect on PH cells compared to Compounds 31 and 32 at lower concentration (<0.5 μM). A growth induction was observed at 10 μM of Compound 31, which is a phenomenon that needs to be explored.

Western Blotting of SATA5, pSTAT5, JAK2 and pJAK2

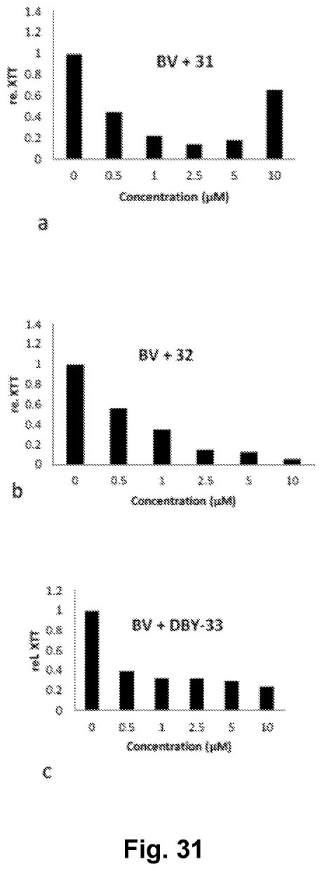

FIG. 31: Concentration-Response (CR) of Compounds 31, 32 and 33 on BV cells. The antiproliferative effect of Compounds 31, 32 and 33 were assessed by exposing BV cells to increasing concertation [0.5 μM-10 0.5 μM]. Cell viability (CV) was assessed using XTT. The of the showed a sharp concentration response effect on PH cells compared to Compound 31 and 32 at lower concentration (<0.5 μM). The effect was almost identical at concentration 1 and 5 μM Compounds 31, 32 and 33. A growth induction was observed at 10 μM of −31.

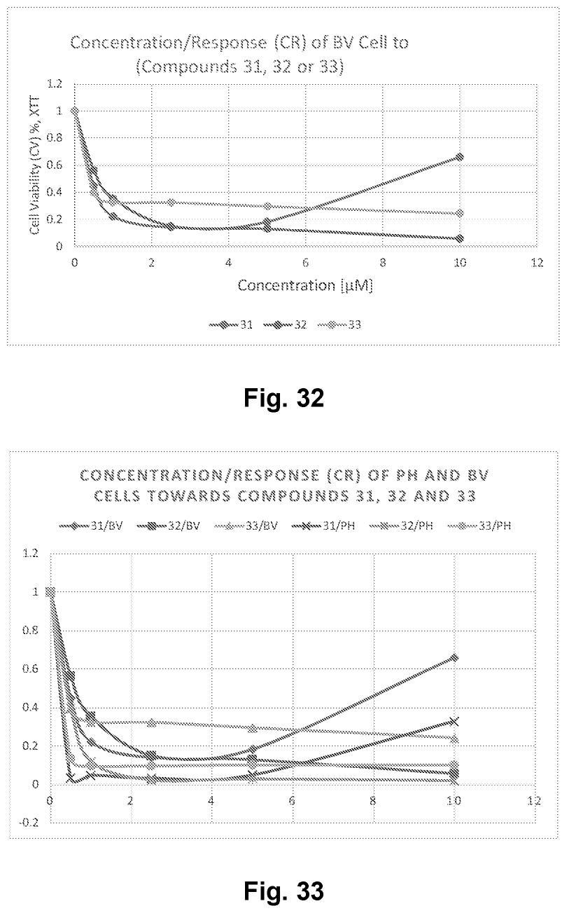

FIG. 32: Concentration-Response (C/R) of Compounds 31, 32 and 33 on BV cells. The antiproliferative effect of Compounds 31, 32 and 33 were assessed by exposing BV cells to increasing concertation [0.5 μM-10.0 μM]. Cell viability was assessed using XTT as described in the experimental part.

FIG. 33: Antiproliferative effect of Compounds 31, 32 and 33 was assessed by exposing patient-derived long-term cell culture system (PD-LTCs) Acute Lymphoblastic Leukemia (ALL) PH and BV cells to increasing concentration [0.5 μM-10.0 μM] of the compound in each case. Cell viability (CV) was assessed using the XTT method. Cell viability ratio (CV %) was calculated by averaging the triplicate, normalizing to inhibition of compound-free vehicle and calculating the ratio.

Following inhibition of cell propagation in ALL PH and BV cells by Compound 31, a growth induction was detected at higher concentration higher than 5 μM indicating some type of duality (inhibition-activation) exerted by Compound 31 concentration-dependent activation. An observation that needs to be explored. Compound 33 showed a sharp concentration-response (C/R) effect on PH cells compared to Compounds 31 and 32 at lower concentration (<0.5 μM). The effect was almost identical at concentrations 1 and 5 μM Compounds 31, 32 and 33.

### Example 14: Effect of Compound 34 against Ph− HEL cells

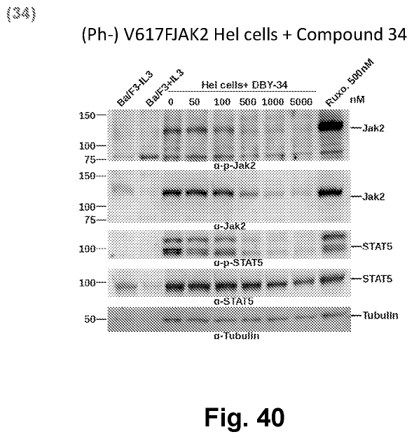

FIG. 40: Western blot analysis of HEL cells using antibodies directed against the indicated proteins. Cells were exposed to increasing concentration of Compound 34 [vehicle (0.0 nM), 50, 100.0, 500.0, 1000.0, and 5000.0 nM].

### Example 15: Comparison of Compound 34, ABL001 and Ruxolitinib

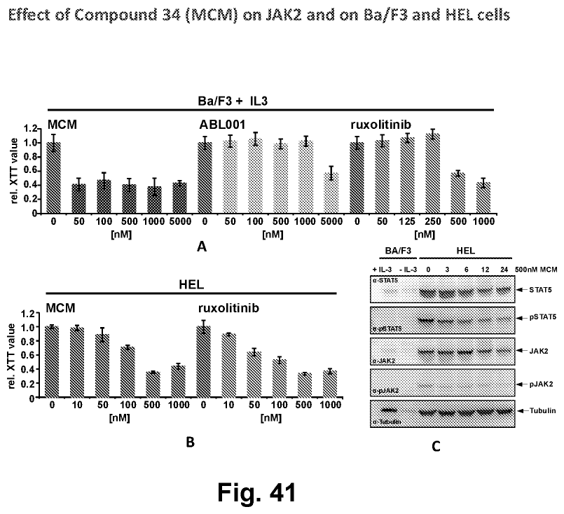

FIG. 41: Concentration-Response (C/R) of Compound 34 against (A) Ph+p185-BCR-ABL-Ba/F3 and (B) JAK2-HEL cells. FIG. 41A depicts the effect of increased concentration of Compound 34 [vehicle (0.0 nM), 50, 100.0, 500.0, 1000.0, and 5000.0 nM] on Ph+ BCR-ABL-Ba/F3 cells compared to Abl001 and Ruxolitinib, FIG. 41B depicts the effect of increased concentration of Compound 34 [vehicle (0.0 nM), 50, 100.0, 500.0, 1000.0, and 5000.0 nM] on Ph− JAK2-HEL cells in comparison with JAK inhibitor Ruxolitinib, and FIG. 41C depicts a Western blot comparison of Abl001 (right), [vehicle (0.0 nM), 50.0, 100.0, 500.0, 1000.0, and 5000.0 nM].

The effect of Compound 34 on signal transducer and activator of transcription (SATA5), phosphorylated STAT5 (pSTAT5), Janus kinase 2 (JAK2) and phosphorylated-JAK2 (pJAK2) was demonstrated by applying 500 nM concentration of the compound on HEL cells. Cells were harvested at the corresponding time intervals (0, 3, 6, 12, and 14 hours) on HELL cells. Anti-STAT5, anti-pSTAT5, anti-JAK2 and anti-pJAK2 antibodies were used. Western blotting revealed the 500 nM concertation was applied on Ba/F3 either in the presence or absence of IL-3 (a cytokine family member shown to induce the Janus kinase (JAK)/signal transducer and activator of transcription (STAT) signaling pathway in several systems). A reduction in the level of expression of SATA5, pSTAT5, JAK2 and pJAK2 was observed when applying 500 nM (see pSTAT5 and pJAK2 at 6 vs 24 hours at FIG. 41 (C)).

### Example 16: Comparison of Compound 34 and ABL001

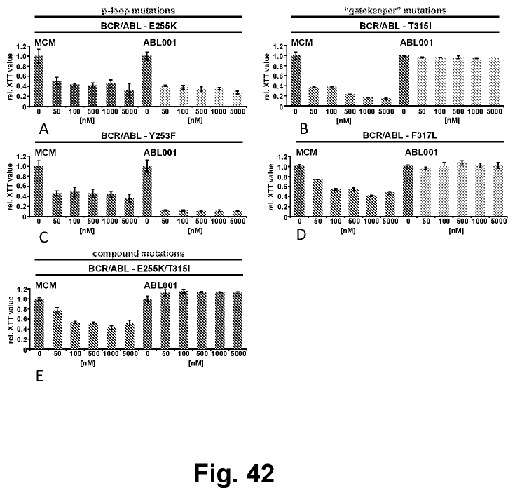

FIG. 42: Concentration-Response (C/R) of Compound 34 against resistant mutants of Ph+ BCR-ABL—Ba/F3 cells. A) Effect of Compound 34 on E255K—BCR-ABL-Ba/F3 cells (left) compared to the clinical candidate Abl001 (right), B) Effect of Compound 34 on the gate keeper mutant T315I-BCR-ABL-Ba/F3 cells (left) compared to the clinical candidate Abl001 (right), C) Effect of Compound 34 on Y253K—BCR-ABL-Ba/F3 cells (left) compared to the clinical candidate Abl001 (right), D) Effect of Compound 34 on F317L-BCR-ABL-Ba/F3 cells (left) compared to the clinical candidate Abl001 (right), E) Effect of Compound 34 on dually mutated E255K/T315I-BCR-ABL-Ba/F3 cells (left) compared to the clinical candidate Abl001 (right). Cells in each case were exposed to increasing concentration of Compound 34 [vehicle (0.0 nM), 50.0, 100.0, 500.0, 1000.0, and 5000.0 nM]. Antiproliferative effect of Compound 34 was assessed by exposing cell lines BCR-ABL-Ba/F3 cells to increasing concentration [vehicle (0.0 μM), 0.5, 1, 2.5, 5, and 10 μM] in each case. Cell viability (CV) was assessed using XTT as described in the experimental part. Cell viability ratio (CV %) was calculated by averaging the triplicate, normalizing to inhibition of compound-free vehicle and calculating the ratio.

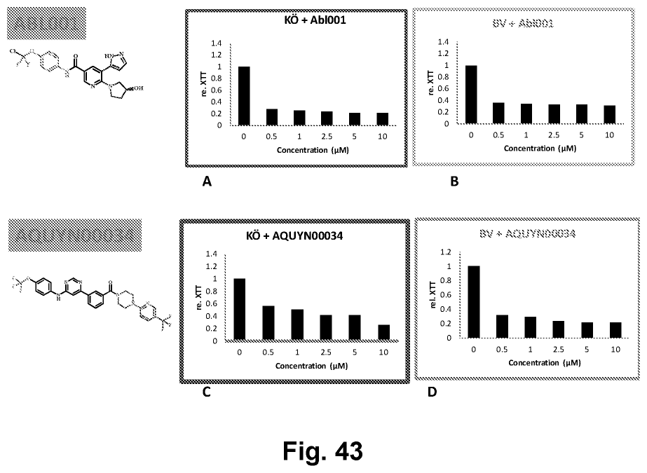

FIG. 43: Concentration-Response (CR) of Compound 34 (below) compared to the clinical candidate Abl001 (above) against KÖ and BV cells. It was noticed that Compound 34 is more effective in inhibiting the proliferation BV than KÖ. The dose response in inhibiting BV was shown in at the lower range of concentrations ([vehicle (0.0 μM), 0.5, 1, 2.5, 5, and 10 μM]). At higher concentrations (>0.5 μM) a plateau was perceived. Cells in each case were exposed to increasing concentration of Compound 34 [vehicle (0.0 μM), 0.5, 1, 2.5, 5, and 10 μM]. Antiproliferative effect of Compound 34 was assessed by exposing cell lines KÖ and BV cells to increasing concertation [vehicle (0.0 nM), 50.0, 100.0, 500.0, 1000.0, 5000.0 and 10000 nM] in each case. Cell viability (CV) was assessed using the XTT as described in the experimental part. Cell viability ratio (CV %) was calculated by averaging the triplicate and normalizing to inhibitory effect of compound-free vehicle.

### Example 17: Drug-Drug Interaction (DDI) Studies: Combination, Analysis and Synergy

### Scoring

The dose-response curves for single agent treatments were generated in Prism 9 (GraphPad). Curve fitting was performed by nonlinear regression using the log(inhibitor) vs. normalized response—Variable slope model.

For drug combinations analysis and synergy score calculation, the SynergyFinder R package was used as described in Zheng et al. [60].

SynergyFinder is an interactive tool for analysing and visualising drug combination dose response data. The input data is a table or matrix that contains the normalized data reported as % viability. The synergy score was calculated based on the Highest Single Agent (HSA) model [61, 64] that states that the expected combination effect equals to the higher effect of individual drugs [60, 62, 63].

The input dose-response matrix is represented as a table where each row contains the information about the one cell in the dose-response matrix [62]. The web-application SynergyFinder was used to preprocess, analyze and visualize pairwise drug combinations in an interactive manner.

### Example 18: Effect of Combining two Inhibitors on Proliferation of PD-LTCs

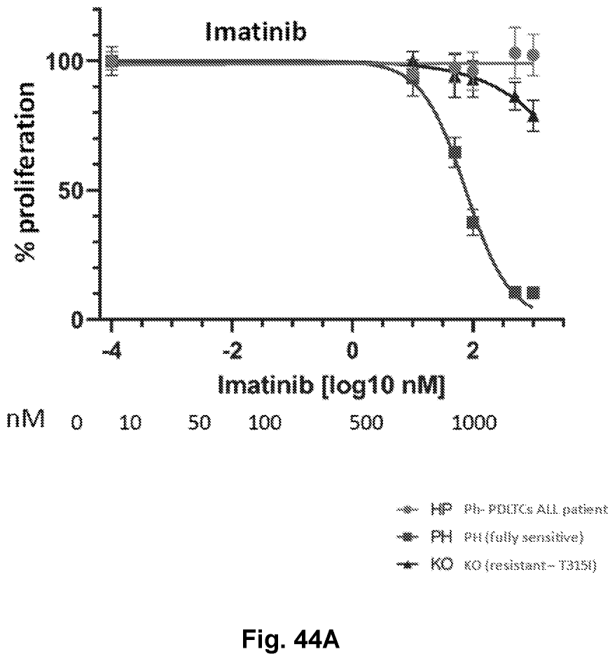

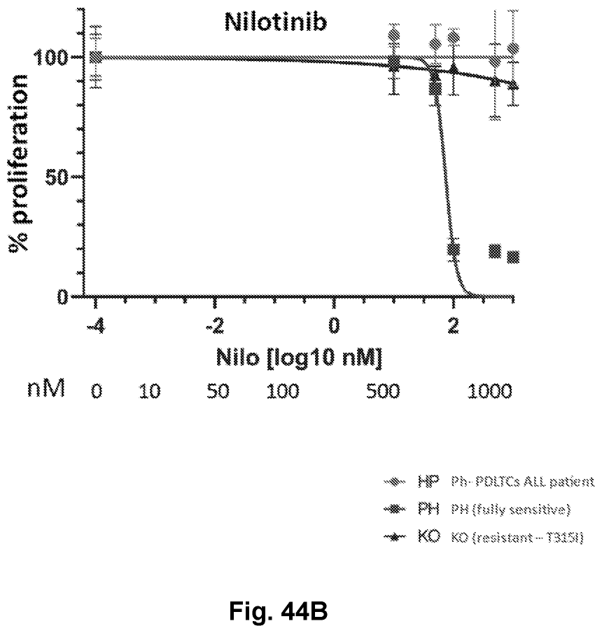

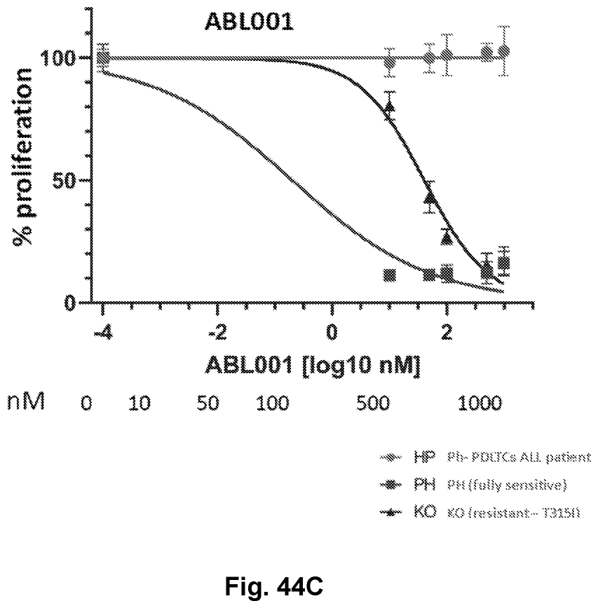

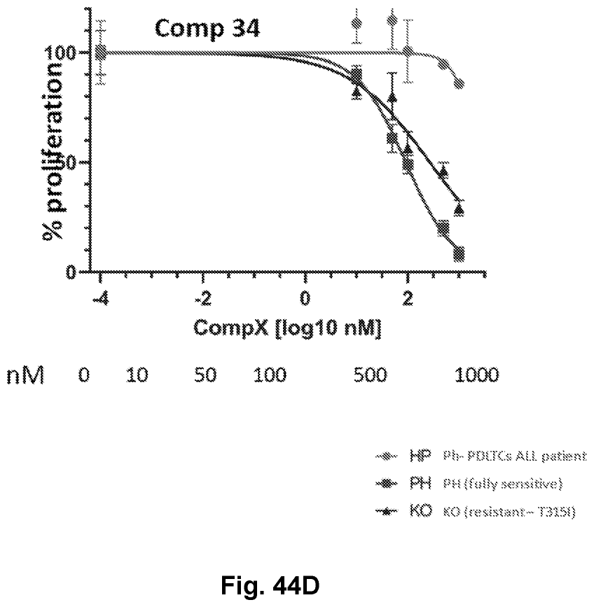

FIGS. 44A-D: Dose-Response Curves for Single Agent Treatment of Imatinib (FIG. 44A), Nilotinib (FIG. 44B), ABL001 (Asciminib) (FIG. 44C), and Compound 34 (FIG. 44D). The cell viability of three Patient-Derived Long-Term Cultures (PD-LTCs): HP (Ph− PD-LTCs) ALL cell that is considered irresponsive towards BCR-ABL targeting agents (circles), PH (Ph+ fully sensitive PD-LTCs) (squares), and KÖ (resistant—T315I, Patient-Derived Long-Term Cultures from Ph+ ALL patient harboring the T315I-p185-BCR-ABL (triangles). Considered TKI-resistant and responsive only to ponatinib or PF114. Ph− HP cells exhibited an irresponsiveness to the applied inhibitors (ATP competitors Imatinib and Nilotinib and to the allosteric inhibitor ABL001) as shown in FIGS. 44A-C(circles). When looking at the responsiveness of HP cells towards Compound 34 one can notice some response at higher doses. On the other hand, the fully sensitive Ph+PD-LTCs PH cells were responsive with IC₅₀ around the 2 nM (squares at FIG. 44) while the T315I-BCR-ABL resistant culture KÖ exhibited lack of response that exceeded 20 folds (extrapolative prediction of the curves) in case Imatinib and Nilotinib. KÖ culture was also resistant to ABL001 at concentrations that were up to 10 folds higher than the IC50 of ABL001 against the sensitive cell. It is only at higher doses that the cells were responding. Compound 34 was effective in inhibiting the Ph+T315I-BCR-ABL KÖ resistant culture in a comparable effectiveness to the sensitive Ph+PH culture.

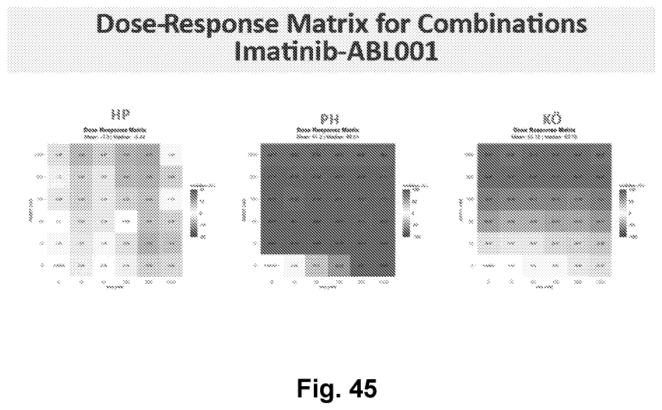

FIG. 45: Dose-Response Matrix for combination of FDA approved Imatinib and ABL001 (Asciminib). Culture in each case (Ph− HP, Ph+PH, or Ph+T315I-BCR-ABL KÖ) was exposed to increasing concentration of either of the drugs while maintaining i.e. increasing the concertation of Imatinib while keeping the concentration of ABL001 was kept constant. Alternatively, the concentration of ABL001 was increased will retaining the concentration of Imatinib constant. The input dose-response matrix is represented as a table where each row contains the information about the one cell in the dose-response matrix [62]. The web-application SynergyFinder was used to preprocess, analyze and visualize pairwise drug combinations in an interactive manner.

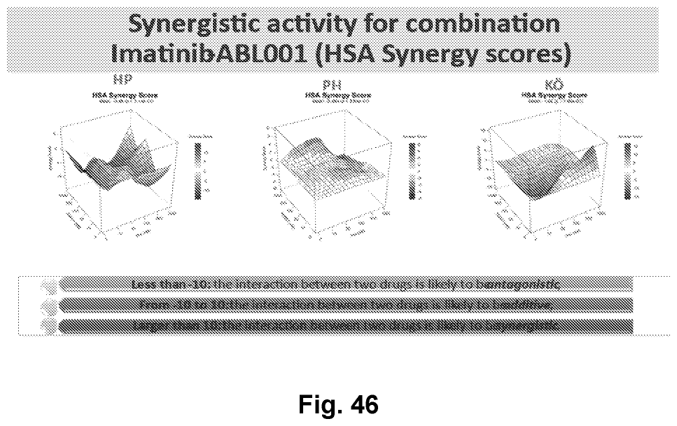

FIG. 46: Synergy score of the combination of Imatinib and ABL001 against three PD-LTCs (Ph− HP, Ph+PH, or Ph+T315I-BCR-ABL KÖ). The HSA Score in Ph− HP culture was ˜4.98 (p 1.1 4e-01) indication a week additive effect. While the HSA Score of the two drugs was ˜0.084 (p=9.642-01) falling in the range −10 to 10 indication a week additive effect. The peak of additivity was with [Imatinib] around 100 nM and [ABL001]˜1000 nM. When both drugs were applied again the resistant Ph+T315I-BCR-ABL KÖ the score was −1.46 (p=7.80e-1) again falling within the week additive effect. The most detected effect was at [Imatinib]˜1000 nM and [ABL001]˜25 nM.

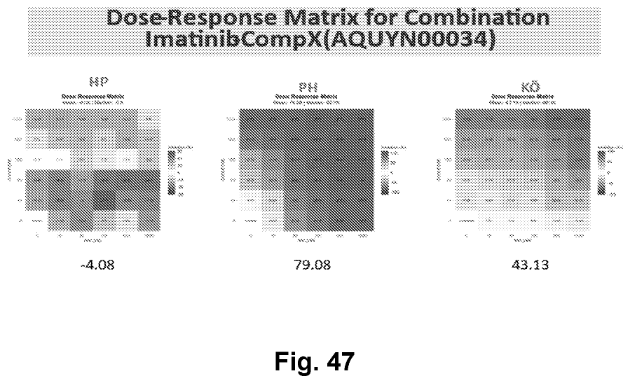

FIG. 47: Dose-Response Matrix for combination of FDA approved Imatinib and Compound 34. Culture in each case (Ph− HP, Ph+PH, or Ph+T315I-BCR-ABL KÖ) was exposed to increasing concentration of either of the drug Imatinib while maintaining the concertation of the second constant i.e., changing the concertation of Imatinib for the starting from 0.0 up to 1000 nM while keeping the concentration of Compound 34 constant. Alternatively, the concentration of Compound 34 was increased from 0.0 up to 1000 nM while retaining the concentration of Imatinib constant.

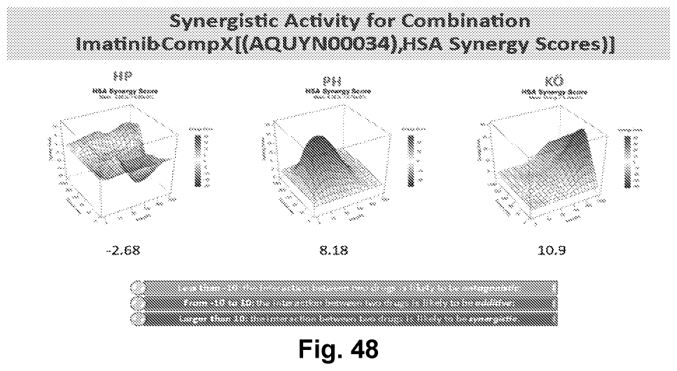

FIG. 48: HSA Synergy score of the combination of Imatinib and Compound 34 against three PD-LTCs (Ph− HP, Ph+PH, or Ph+T315I-BCR-ABL KÖ). The HSA Score in Ph− HP culture was −2.68 (p 6.95e-01) indication a week additive effect. While the HSA Score of the two drugs in Ph+PH sensitive culture was 8.18 (p=2.75e-01) falling in the range of (−10 to 10) indication a strong additive effect against such type of culture. The peak of additivity was with [Imatinib] around 100 nM and [34]˜1000 nM. When both drugs were applied again the resistant Ph+T315I-BCR-ABL KÖ the score was 10.9 (p=3.15e-1) Indicating a synergistic interaction between the two inhibitors. The most detected effect was at [Imatinib]˜1000 nM and [34]˜50 nM. Such interaction is indicative for reversal of resistant of BCR-ABL dependent cells to FDA approved and generic Imatinib.

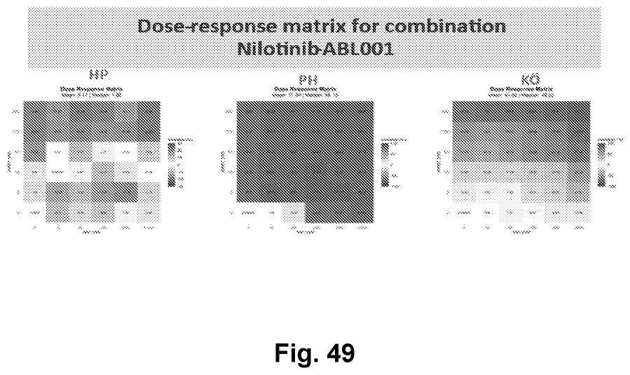

FIG. 49: Dose-Response Matrix for combination of FDA approved drugs Nilotinib and ABL001 (Asciminib). Culture in each case (Ph− HP, Ph+PH, or Ph+T315I-BCR-ABL KÖ) was exposed to increasing concentration of either of the drugs while maintaining i.e., increasing the concertation of Nilotinib (range 0.0 until 1000 nM) while keeping the concentration of ABL001 (Asciminib) was kept constant. Alternatively, the concentration of ABL001 was increased (range 0.0 until 1000 nM) while retaining the concentration of Nilotinib constant. The input dose-response matrix is represented as a table where each row contains the information about the one cell in the dose-response matrix [62]. The web-application SynergyFinder was used to preprocess, analyze and visualize pairwise drug combinations in an interactive manner.

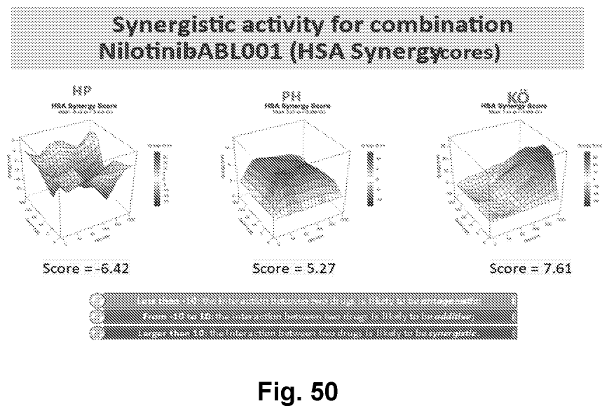

FIG. 50: HSA Synergy score of the combination of Nilotinib and ABL001 against three PD-LTCs (Ph− HP, Ph+PH, or Ph+T315I-BCR-ABL KÖ). The HSA Score in Ph− HP culture was −6.42 (p 3.30e-01) indication a week additive effect. While the HSA Score of the two drugs in Ph+PH sensitive culture was 5.27 (p=9.29-03) falling in the range of (−10 to 10) indication a strong additive effect against such type of culture. The additive effect of ABL001 and Nilotinib spans over a range of concentrations. The peak of additivity was with [Nilotinib] around 100 nM and [ABL001]˜1000 nM. When both drugs were applied again the resistant Ph+T315I-BCR-ABL KÖ the score was 7.61 (p=4.86e-1) Indicating a synergistic interaction between the two inhibitors. The most detected effect was at [Nilotinib]˜1000 nM and [ABL0014]˜50 nM. Though the interaction between ABL001 and Nilotinib is additive against the two culture (Ph+PH and Ph+T315I-BCR-ABL KÖ) there was as light sharper impact (˜40% enhancement) against the resistant culture KÖ. Such interaction is indicative for that ability of allosteric inhibitors in sensitizing cells towards FDA approved drugs ending in reversal of resistant BCR-ABL dependent cells towards such drugs. That can gain significance as fewer toxic side effects are expected to immerge.

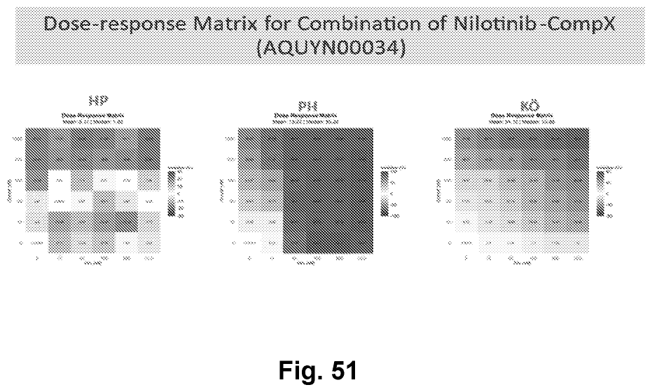

FIG. 51: Dose-Response Matrix for combination of FDA approved drugs Nilotinib and the investigational Compound 34 against three PD-LTCs (Ph− HP, Ph+PH, or Ph+T315I-BCR-ABL KÖ). Culture in each case (Ph− HP, Ph+PH, or Ph+T315I-BCR-ABL KÖ) was exposed to increasing concentration of either of the drugs while maintaining i.e., increasing the concertation of Nilotinib (range 0.0 until 1000 nM) while keeping the concentration of Compound 34 was kept constant. Alternatively, the concentration of Compound 34 was increased (range 0.0 until 1000 nM) while retaining the concentration of Nilotinib constant. The input dose-response matrix is represented as a table where each row contains the information about the one cell in the dose-response matrix [62]. The web-application SynergyFinder was used to preprocess, analyze and visualize pairwise drug combinations in an interactive manner.

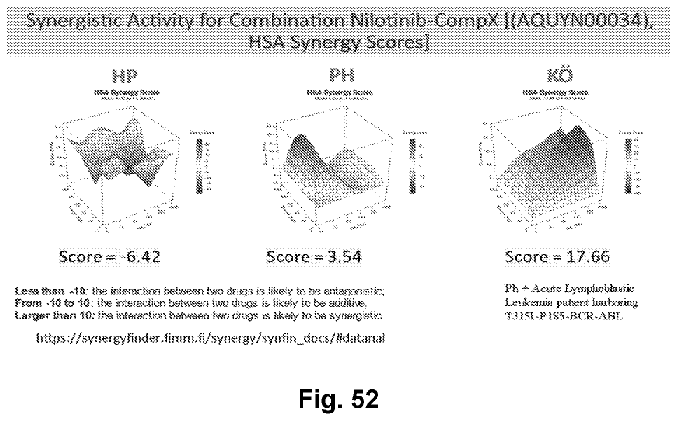

FIG. 52: HSA Synergy score of the combination of Nilotinib and Compound 34 against three PD-LTCs (Ph− HP, Ph+PH, or Ph+T315I-BCR-ABL KÖ). The HSA Score in Ph− HP culture was −6.42 (p 3.30e-01) indication a week additive effect. While the HSA Score of the two drugs in Ph+PH sensitive culture was 3.54 (p=6.34-01) falling in the range of (score=−10 to 10) indication a a week additive effect against such type of culture. The additive effect of Compound 34 and Nilotinib was noticed at lower concentrations of Nilotinib and started to be detected at low concertation of Compound 34 as low as 10 nm. The additive interaction is strengthened upon the increase of [Compound 34] and reach a peak at 1000 nM. When both drugs were applied again the resistant KÖ (Ph+T315I-p185-BCR-ABL), the score was 17.66 (p=6.01e-2) indicating a profound synergistic interaction between the two inhibitors. The strongest synergism was detected when [Nilotinib]˜1000 nM and [Compound 34]˜50 nM. The interaction between Compound 34 and Nilotinib was additive against the culture Ph+PH and strong synergistic against the resistant Ph+T315I-BCR-ABL KÖ. Comparing the two scores is showed an augmentation by a factor of 5.0. Such result underlines that ability of the allosteric inhibitor Compound 34 to chemosensitize resistant cells to Nilotinib, a second-generation FDA approved ABL inhibitors.

Among the combination tested so far, Compound 34 has the strongest synergistic interaction with Nilotinib against T315I-BCR-ABL KÖ culture.

It is obvious that the foregoing embodiments of the invention are examples and can be varied in many ways. Such present or future variations are not to be regarded as a departure from the spirit and scope of the invention, and all such modifications as would be obvious to one skilled in the art are intended to be included within the scope of the following claims.

### REFERENCES

1. Hochhaus A, Kreil S, Corbin A S, La Ros6e P, Müller MC, Lahaye T, et al. Molecular and chromosomal mechanisms of resistance to imatinib (ST1571) therapy. Leukemia. 2002; 16(11):2190-6.
2. Daver N, Cortes J, Ravandi F, Patel K P, Burger J A, Konopleva M, et al. Secondary mutations as mediators of resistance to targeted therapy in leukemia. Blood. 2015; 125(21):3236-45.
3. Melchor L, Brioli A, Wardell C P, Murison A, Potter N E, Kaiser M F, et al. Single-cell genetic analysis reveals the composition of initiating clones and phylogenetic patterns of branching and parallel evolution in myeloma. Leukemia. 2014; 28(8):1705-15.
4. Jabbour E, Kantarjian H. Chronic myeloid leukemia: 2018 update on diagnosis, therapy and monitoring. American Journal of Hematology. 2018; 93(3):442-59.
5. Weisberg E, Manley P W, Cowan-Jacob S W, Hochhaus A, Griffin J D. Second generation inhibitors of BCR-ABL for the treatment of imatinib-resistant chronic myeloid leukaemia. Nature Reviews Cancer. 2007.
6. Wehrle J, von Bubnoff N. Ponatinib: A third-generation inhibitor for the treatment of CML. In: Recent Results in Cancer Research. 2018. p. 109-18.
7. BrQmmendorf T H, Gontarewicz A. Danusertib (formerly PHA-739358)—A novel combined Pan-Aurora kinases and third generation Bcr-Abl tyrosine kinase inhibitor. Vol. 184, Recent Results in Cancer Research. 2010. p. 199-214.
8. Loren C P, Aslan J E, Rigg R A, Nowak M S, Healy L D, Gruber A, et al. The BCR-ABL inhibitor ponatinib inhibits platelet immunoreceptor tyrosine-based activation motif (ITAM) signaling, platelet activation and aggregate formation under shear. Thrombosis Research. 2015; 135(1):155-60.
9. Dorer D J, Knickerbocker R K, Baccarani M, Cortes J E, Hochhaus A, Talpaz M, et al. Impact of dose intensity of ponatinib on selected adverse events: Multivariate analyses from a pooled population of clinical trial patients. Leukemia Research. 2016; 48:84-91.
10. Caldemeyer L, Dugan M, Edwards J, Akard L. Long-Term Side Effects of Tyrosine Kinase Inhibitors in Chronic Myeloid Leukemia. Vol. 11, Current Hematologic Malignancy Reports. 2016. p. 71-9.
11. Samanta A K, Chakraborty S N, Wang Y, Schlette E, Premkumar Reddy E, Arlinghaus R B. Destabilization of Bcr-Abl/Jak2 network by a Jak2/Abl kinase inhibitor ON044580 overcomes drug resistance in blast crisis chronic myelogenous leukemia (CML). Genes and Cancer. 2010; 1(4):346-59.
12. Jatiani S S, Cosenza S C, Ramana Reddy M V., Ha J H, Baker S J, Samanta A K, et al. A non-ATP-competitive dual inhibitor of JAK2V617F and BCR-ABLT315I kinases: Elucidation of a novel therapeutic spectrum based on substrate competitive inhibition. Genes and Cancer. 2010; 1(4):331-45.
13. Rives S, Estella J, Gömez P, Löpez-Duarte M, de Miguel P G, Verdeguer A, et al. Intermediate dose of imatinib in combination with chemotherapy followed by allogeneic stem cell transplantation improves early outcome in paediatric Philadelphia chromosome-positive acute lymphoblastic leukaemia (ALL): Results of the Spanish Cooperative G. British Journal of Haematology. 2011; 154(5):600-11.
14. Refaat H M. Synthesis and anticancer activity of some novel 2-substituted benzimidazole derivatives. European Journal of Medicinal Chemistry. 2010; 45(7):2949-56.
15. Ren R. Mechanisms of BCR-ABL in the pathogenesis of chronic myelogenous leukaemia. Vol. 5, Nature Reviews Cancer. 2005. p. 172-83.
16. Srinivasan D, Plattner R. Activation of Abl tyrosine kinases promotes invasion of aggressive breast cancer cells. Cancer Research. 2006; 66(11):5648-55.
17. Gur Z T, Qali§ kan B, Banoglu E. Drug discovery approaches targeting 5-lipoxygenase-activating protein (FLAP) for inhibition of cellular leukotriene biosynthesis. Vol. 153, European Journal of Medicinal Chemistry. 2018. p. 34-48.
18. Lehmann B D, Bauer J A, Chen X, Sanders M E, Chakravarthy A B, Shyr Y, et al.

Identification of human triple-negative breast cancer subtypes and preclinical models for selection of targeted therapies. Journal of Clinical Investigation. 2011; 121(7):2750-67.
19. Lin J, Arlinghaus R. Activated c-Abl tyrosine kinase in malignant solid tumors. Vol. 27, Oncogene. 2008. p. 4385-91.
20. Schlatterer S D, Acker C M, Davies P. C-Abl in neurodegenerative disease. In: Journal of Molecular Neuroscience. 2011. p. 445-52.
21. Mahul-Mellier A L, Fauvet B, Gysbers A, Dikiy I, Oueslati A, Georgeon S, et al. C-Abl phosphorylates α-synuclein and regulates its degradation: Implication for α-synuclein clearance and contribution to the pathogenesis of parkinson's disease. Human Molecular Genetics. 2014; 23(11):2858-79.
22. Khatri A, Wang J, Pendergast A M. Multifunctional Abl kinases in health and disease. Journal of Cell Science. 2016; 129(1):9-16.
23. Klein A, Maldonado C, Vargas L M, Gonzalez M, Robledo F, Perez de Arce K, et al. Oxidative stress activates the c-Abl/p73 proapoptotic pathway in Niemann-Pick type C neurons. Neurobiology of Disease. 2011; 41(1):209-18.
24. Schlatterer S D, Tremblay M A, Acker C M, Davies P. Neuronal c-Abl overexpression leads to neuronal loss and neuroinflammation in the mouse forebrain. Journal of Alzheimer's Disease. 2011; 25(1):119-33.
25. Zhou Z H, Wu Y F, Wang X min, Han Y Z. The c-Abl inhibitor in Parkinson disease. Vol. 38, Neurological Sciences. 2017. p. 547-52.
26. Jabbour E, Kantarjian H. Chronic myeloid leukemia: 2020 update on diagnosis, therapy and monitoring. American journal of hematology. 2020 June; 95(6):691-709.
27. Ernst T, Obstfelder E, Hochhaus A. Chronic myeloid leukemia. Onkologe. 2018; 24(5):427-42.
28. Savona M, Talpaz M. Getting to the stem of chronic myeloid leukaemia. Vol. 8, Nature Reviews Cancer. 2008. p. 341-50.
29. Saussele S, Richter J, Guilhot J, Gruber F X, Hjorth-Hansen H, Almeida A, et al.

Discontinuation of tyrosine kinase inhibitor therapy in chronic myeloid leukaemia (EURO-SKI): a prespecified interim analysis of a prospective, multicentre, non-randomised, trial. The Lancet Oncology. 2018; 19(6):747-57.
30. Alikian M, Gale R P, Apperley J F, Foroni L, Alikian M. Molecular techniques for the personalised management of patients with chronic myeloid leukaemia. Vol. 11, Biomolecular Detection and Quantification. 2017. p. 4-20.
31. Ottmann O G, Pfeifer H. Management of Philadelphia chromosome-positive acute lymphoblastic leukemia (Ph+ ALL). Hematology/the Education Program of the American Society of Hematology American Society of Hematology Education Program. 2009; 371-81.
32. Bernt K M, Hunger S P. Current Concepts in Pediatric Philadelphia Chromosome-Positive Acute Lymphoblastic Leukemia. Frontiers in Oncology. 2014; 4.
33. Tran T H, Loh M L. Ph-like acute lymphoblastic Leukemia. Hematology. 2016; 2016(1):561-6.
34. Ghia P, Ferreri A M, Galigaris-Cappio F. Chronic lymphocytic leukemia. Vol. 64, Critical Reviews in Oncology/Hematology. 2007. p. 234-46.
35. Rowe J M. Optimal management of adults with ALL. Vol. 144, British Journal of Haematology. 2009. p. 468-83.
36. Ribera J M. Optimal approach to treatment of patients with Philadelphia chromosome-positive acute lymphoblastic leukemia: How to best use all the available tools. Vol. 54, Leukemia and Lymphoma. 2013. p. 21-7.
37. Moslehi J J, Deininger M. Tyrosine kinase inhibitor-associated cardiovascular toxicity in chronic myeloid leukemia. Vol. 33, Journal of Clinical Oncology. 2015. p. 4210-8.
38. Zhang J, Adrien F J, Jahnke W, Cowan-Jacob S W, Li A G, lacob R E, et al. Targeting Bcr-Abl by combining allosteric with ATP-binding-site inhibitors. Nature. 2010; 463(7280):501-6.
39. Adrien F J, Ding Q, Sim T, Velentza A, Sloan C, Liu Y, et al. Allosteric inhibitors of Bcr-abl-dependent cell proliferation. Nature Chemical Biology. 2006; 2(2):95-102.
40. Fallacara A L, Tintori C, Radi M, Schenone S, Botta M. Insight into the allosteric inhibition of Abl kinase. Journal of Chemical Information and Modeling. 2014; 54(5):1325-38.
41. Hassan A Q, Sharma S V., Warmuth M. Allosteric inhibition of BCR-ABL. Vol. 9, Cell Cycle. 2010. p. 3710-4.
42. Deng X, Okram B, Ding Q, Zhang J, Choi Y, Adrien F J, et al. Expanding the diversity of allosteric Bcr-Abl inhibitors. Journal of Medicinal Chemistry. 2010; 53(19):6934-46.
43. Krause D S, Van Etten R A. Tyrosine Kinases as Targets for Cancer Therapy. New England Journal of Medicine. 2005; 353(2):172-87.
44. Gotink K J, Verheul H M W. Anti-angiogenic tyrosine kinase inhibitors: What is their mechanism of action?Vol. 13, Angiogenesis. 2010. p. 1-14.
45. Karaman $, Küçücük S. Neurological metastases. In: Breast Disease: Management and Therapies. 2016. p. 635-60.
46. Wang Q, Zorn J A, Kuriyan J. A structural atlas of kinases inhibited by clinically approved drugs. In: Methods in Enzymology. 2014. p. 23-67.
47. Milojkovic D, Apperley J F. Mechanisms of resistance to imatinib and second-generation tyrosine inhibitors in chronic myeloid leukemia. Vol. 15, Clinical Cancer Research. 2009. p. 7519-27.
48. da Cunha Santos G, Shepherd F A, Tsao M S. EGFR Mutations and Lung Cancer. Annual Review of Pathology: Mechanisms of Disease. 2011; 6(1):49-69.
49. Small D. FLT3 mutations: biology and treatment. Hematology/the Education Program of the American Society of Hematology. American Society of Hematology. Education Program. 2006. p. 178-84.
50. Cortes J, Jabbour E, Kantarjian H, Yin C C, Shan J, O'Brien S, et al. Dynamics of BCR-ABL kinase domain mutations in chronic myeloid leukemia after sequential treatment with multiple tyrosine kinase inhibitors. Blood. 2007; 110(12):4005-11.
51. Soverini S, Hochhaus A, Nicolini F E, Gruber F, Lange T, Saglio G, et al. BCR-ABL kinase domain mutation analysis in chronic myeloid leukemia patients treated with tyrosine kinase inhibitors: Recommendations from an expert panel on behalf of European LeukemiaNet. Vol. 118, Blood. 2011. p. 1208-15.
52. Williams A B, Nguyen B, Li L, Brown P, Levis M, Leahy D, et al. Mutations of FLT3/ITD confer resistance to multiple tyrosine kinase inhibitors. Leukemia. 2013; 27(1):48-55.
53. Afsar Ali Mian, Anna Metodieva, Susanne Badura, Mamduh Khateb, Nili Ruimi, Yousef Najajreh, Oliver Gerhard Ottmann, Jamal Mahajna, Martin Ruthardt, Allosteric inhibition enhances the efficacy ofABL kinase inhibitors to target unmutated BCR-ABL and BCR-ABL-T315I, BMC Cancer. 2012 Sep. 17; 12:411
54. Mian A A, Oancea C, Zhao Z, Ottmann O G, Ruthardt M., Oligomerization inhibition, combined with allosteric inhibition, abrogates the transformation potential of T315I-positive BCR-ABL. Leukemia 2009; 23: 2242-2247.
55. Quentmeier, H., MacLeod, R. A., Zaborski, M., Drexler, H. G. (2006). JAK2 V617F tyrosine kinase mutation in cell lines derived from myeloproliferative disorders. Leukemia 20 (3): 471-476
56. Mian, A., Rafiei, A., Haberbosch, I. et al. PF-114, a potent and selective inhibitor of native and mutated BCR/ABL is active against Philadelphia chromosome-positive (Ph+) leukemias harboring the T315I mutation. Leukemia 29, 1104-1114 (2015). https://doi.org/l0.1038/leu.2014.326
57. Mian A A, Metodieva A, Najajreh Y, Ottmann O G, Mahajna J, Ruthardt M. p185(BCR/ABL) has a lower sensitivity than p210(BCR/ABL) to the allosteric inhibitor GNF-2 in Philadelphia chromosome-positive acute lymphatic leukemia, Haematologica. 2012 February; 97(2):251-7 (and references cited therein)
58. Badura S, Tesanovic T, Pfeifer H, Liebermann M, Falkenburg J H F, Ruthardt M, Ottmann O G: Differential effects of selective inhibitors targeting the PI3K/AKT/mTOR pathway in long-term cultures of acute lymphoblastic leukemia reveal a distinct role of mTORC2. submitted for publication
59. Nijmeijer B A, Szuhai K, Goselink H M, van Schie M L, van der Burg M, de Jong D, Marijt E W, Ottmann O G, Willemze R, Falkenburg J H: Long-term culture of primary human lymphoblastic leukemia cells in the absence of serum or hematopoietic growth factors.

Exp Hematol. 2009, 37 (3): 376-385. 10.1016/j.exphem.2008.11.002.
60. Zheng, S.; Wang, W.; Aldahdooh, J.; Malyutina, A.; Shadbahr, T.; Pessia, A.; Jing, T.

SynergyFinder Plus: towards a better interpretation and annotation of drug combination screening datasets. bioRxiv 2021.06.01.446564 (2021) doi:10.1101/2021.06.01.446564
61. Berenbaum M C. What is synergy?Pharmacol Rev. 1989 June; 41(2):93-141. Erratum in: Pharmacol Rev 1990 September; 41(3):422. PMID: 2692037
62. Aleksandr lanevski, Anil K Giri, Tero Aittokallio, SynergyFinder 2.0: visual analytics of multi-drug combination synergies, Nucleic Acids Research, Volume 48, Issue W1, 2 Jul. 2020, Pages W488-W493, https://doi.org/10.1093/nar/gkaa216
63. Aleksandr lanevski, Liye He, Tero Aittokallio, Jing Tang, SynergyFinder: a web application for analyzing drug combination dose-response matrix data, Bioinformatics, Volume 33, Issue 15, 1 Aug. 2017, Pages 2413-2415, https://doi.org//10.1093/bioinformatics/btx162
64. Foucquier J, Guedj M. Analysis of drug combinations: current methodological landscape, Pharmacol Res Perspect. 2015; 3(3):e00149. doi:10.1002/prp2.149 [published correction appears in Pharmacol Res Perspect. 2019 December; 7(6):e00549].

1. A compound having the structural Formula (I): 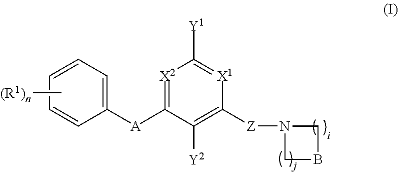
or a pharmaceutically acceptable salt thereof,
wherein:
X¹ and X² are each independently N or CH;
A is —NH—, —N(CH₃)—, —N(OH)—, —N(OCH₃)—, —NHC(O)—, —OC(O)NH—, —NHC(O)NH—, —NHS(O)₂—, —NHS(O)₂NH—, —S—, —O—, or —CH₂—, C(O)—, —S(O)—, or —S(O)₂—;
Z is absent, —NH—, —N(CH₃)—, —N(OH)—, —N(OCH₃)—, —C(O)—, —SO₂—, —NHC(O)—, —NH(CO)NH—, —NHS(O)₂—, —NHS(O)₂NH—, —CH₂—, —SO—, a benzoyl moiety, or a phenyl moiety,
Y₁ and Y₂ are each independently hydrogen, halogen, C₁₋₁₆alkyl, OH, —O—C₁₋₁₆alkyl, —S—C₁₋₁₆alkyl, —S(O)—C₁₋₁₆alkyl, —S(O₂)—C₁₋₁₆alkyl, —C(O)—C₁₋₁₆alkyl, a cycloalkyl group, a heterocyclic group, a heteroaromatic group, or an aryl group, wherein the —C₁₋₁₆alkyl, —O—C₁₋₁₆alkyl, cycloalkyl group, S—C₁₋₁₆alkyl, —S(O)—C₁₋₁₆alkyl, —S(O₂)—C₁₋₁₆alkyl, —C(O)—C₁₋₁₆alkyl, heterocyclic group, heteroaromatic group, and aryl group are optionally substituted with one or more substituents independently selected from halogen, hydroxyl, oxo, lower alkyl, lower alkoxyl, and phenyl;
B is —NR² or —CHR²;
R² is —Y—R³;
Y is absent, —C(O)—, SO—SO₂—, —(CO)NH—;
R³ is hydrogen, —C₁₋₁₆alkyl, —O—C₁₋₁₆alkyl, —S—C₁₋₁₆alkyl, —S(O)—C₁₋₁₆alkyl, —S(O)₂—C₁₋₁₆alkyl, —C(O)—C₁₋₁₆alkyl, a cycloalkyl group, a heterocyclic group, a heteroaromatic group, or an aryl group, wherein the —C₁₋₁₆alkyl, —O—C₁₋₁₆alkyl, cycloalkyl group, S—C₁₋₁₆alkyl, —S(O)—C₁₋₁₆alkyl, —S(O)₂—C₁₋₁₆alkyl, —C(O)—C₁₋₁₆alkyl, heterocyclic group, heteroaromatic group, and aryl group are optionally substituted with one or more substituents independently selected from halogen, hydroxyl, oxo, lower alkyl, lower alkoxyl, halogenated lower alkyl, halogenated lower alkoxy, and phenyl;
i is 1, 2, 3 or 4;
j is 1, 2, 3 or 4;
R¹ is halogen, hydroxyl, —C₁₋₁₆alkyl, —O—C₁₋₁₆alkyl, S—C₁₋₁₆alkyl, —S(O)—C₁₋₁₆alkyl, —S(O)₂—C₁₋₁₆ alkyl, —C(O)—C₁₋₁₆alkyl or —N(R^{b})₂, wherein R^{b} is —H or —C₁₋₆alkyl, wherein each alkyl is optionally substituted with one or more halogens selected from F, Cl and Br; and
n is 0, 1, 2 or 3,
wherein the compound is other than N-phenyl-6-(4-phenylpiperazin-1-yl)pyrimidin-4-amine, N-phenyl-6-(4-piperazin-1-yl)pyrimidin-4-amine, and N-phenyl-6-piperidin-1-ylpyrimidin-4-amine.

2. A compound as defined in claim 1, wherein the compound has the structural Formula (II): 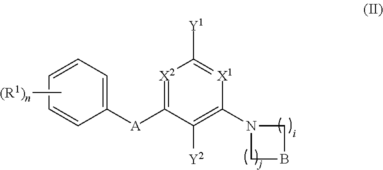
or a pharmaceutically acceptable salt thereof,
wherein:
X¹ and X² are each independently N or CH;
A is —NH—, —N(CH₃)—, —N(OH)—, —N(OCH₃)—, —NHC(O)—, —OC(O)NH—, —NHC(O)NH—, —NHS(O)₂—, —NHS(O)₂NH—, —S—, —O—, or —CH₂—, C(O)—, —S(O)—, or —S(O)₂—;
Y¹ and Y² are independently hydrogen, halogen, C₁₋₁₆alkyl, —O—C₁₋₁₆alkyl, —S—C₁₋₁₆alkyl, —S(O)—C₁₋₁₆alkyl, —S(O)₂—C₁₋₁₆alkyl, —C(O)—C₁₋₁₆alkyl, a cycloalkyl group, a heterocyclic group, a heteroaromatic group, or an aryl group, wherein the —C₁₋₁₆alkyl, —O—C₁₋₁₆alkyl, cycloalkyl group, —S—C₁₋₁₆alkyl, —S(O)—C₁₋₁₆alkyl, —S(O)₂—C₁₋₁₆alkyl, —C(O)—C₁₋₁₆alkyl, heterocyclic group, heteroaromatic group, and aryl group are optionally substituted with one or more substituents independently selected from halogen, hydroxyl, oxo, lower alkyl, lower alkoxyl, and phenyl;
B is —NR² or —CHR²;
R² is —Y—R³;
Y is absent, —C(O)—, —SO—, —SO₂—, or —(CO)NH—;
R³ is hydrogen, —C₁₋₁₆alkyl group, —O—C₁₋₁₆alkyl group, —S—C₁₋₁₆alkyl group, —S(O)—C₁₋₁₆alkyl group, —S(O)₂—C₁₋₁₆alkyl group, —C(O)—C₁₋₁₆alkyl group, a cycloalkyl group, a heterocyclic group, a heteroaromatic group, or an aryl group, wherein the —C₁₋₁₆alkyl group, —O—C₁₋₁₆alkyl group, cycloalkyl group, S—C₁₋₁₆alkyl group, —S(O)—C₁₋₁₆alkyl group, —S(O)₂—C₁₋₁₆alkyl group, —C(O)—C₁₋₁₆alkyl group, heterocyclic group, heteroaromatic group, and aryl group are optionally substituted with one or more substituents independently selected from halogen, hydroxyl, oxo, lower alkyl, lower alkoxyl, halogenated lower alkyl, halogenated lower alkoxy, and phenyl;
i is 1, 2, 3 or 4;
j is 1, 2, 3 or 4;
R¹ is halogen, hydroxyl, —C₁₋₁₆alkyl, —O—C₁₋₁₆alkyl, S—C₁₋₁₆alkyl group, —S(O)—C₁₋₁₆alkyl group, —S(O₂)—C₁₋₁₆alkyl group, —C(O)—C₁₋₁₆alkyl group or —N(R^{b})₂, wherein R^{b} is —H or —C₁₋₆alkyl, wherein each alkyl is optionally substituted with one or more halogens selected from F, Cl and Br; and
n is 0, 1, 2 or 3,
wherein the compound is other than N-phenyl-6-(4-phenylpiperazin-1-yl)pyrimidin-4-amine, N-phenyl-6-(4-piperazin-1-yl)pyrimidin-4-amine, and N-phenyl-6-piperidin-1-ylpyrimidin-4-amine.

3. A compound as defined in claim 1, wherein the compound has the structural Formula (III): 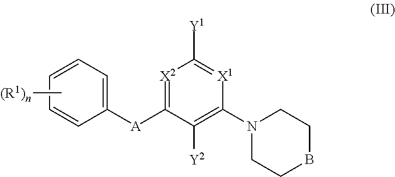
or a pharmaceutically acceptable salt thereof,
wherein:
X¹ and X² are each independently N or CH;
A is —NH—, —N(CH₃)—, —N(OH)—, —N(OCH₃)—, —NHC(O)—, —OC(O)NH—, —NHC(O)NH—, —NHS(O)₂—, —NHS(O)₂NH—, —S—, —O—, or —CH₂—, C(O)—, —S(O)—, or —S(O)₂—;
Y¹ and Y² are independently hydrogen, halogen, —C₁₋₁₆alkyl, —O—C₁₋₁₆alkyl, —S—C₁₋₁₆alkyl, —S(O)—C₁₋₁₆alkyl, —S(O)₂—C₁₋₁₆alkyl, —C(O)—C₁₋₁₆alkyl, a cycloalkyl group, a heterocyclic group, a heteroaromatic group, or an aryl group, wherein the —C₁₋₁₆alkyl, —O—C₁₋₁₆alkyl, cycloalkyl group, S—C₁₋₁₆alkyl, —S(O)—C₁₋₁₆alkyl, —S(O)₂—C₁₋₁₆alkyl, —C(O)—C₁₋₁₆alkyl, heterocyclic group, heteroaromatic group, and aryl group are optionally are optionally substituted with one or more substituents independently selected from halogen, hydroxyl, oxo, lower alkyl, lower alkoxyl, and phenyl;
B is —NR² or —CHR²;
R² is —Y—R³;
Y is absent, —C(O)—, —SO—, —SO₂—, or —(CO)NH—;
R³ is hydrogen, —C₁₋₁₆alkyl, —O—C₁₋₁₆alkyl, —S—C₁₋₁₆alkyl, —S(O)—C₁₋₁₆alkyl, —S(O)₂—C₁₋₁₆alkyl, —C(O)—C₁₋₁₆alkyl, a cycloalkyl group, a heterocyclic group, a heteroaromatic group, or an aryl group, wherein the —C₁₋₁₆alkyl, —O—C₁₋₁₆alkyl, cycloalkyl group, S—C₁₋₁₆alkyl, —S(O)—C₁₋₁₆alkyl, —S(O)₂—C₁₋₁₆alkyl, —C(O)—C₁₋₁₆alkyl, heterocyclic group, heteroaromatic group, and aryl group are optionally substituted with one or more substituents independently selected from halogen, hydroxyl, oxo, lower alkyl, lower alkoxyl, halogenated lower alkyl, halogenated lower alkoxy, and phenyl;
R¹ is halogen, hydroxyl, —C₁₋₁₆alkyl, —O—C₁₋₁₆alkyl, S—C₁₋₁₆alkyl, —S(O)—C₁₋₁₆alkyl, —S(O)₂—C₁₋₁₆ alkyl, —C(O)—C₁₋₁₆alkyl or —N(R^{b})₂, wherein R^{b} is —H or —C₁₋₆alkyl, wherein each alkyl is optionally substituted with one or more halogens selected from F, Cl and Br; and
n is 0, 1, 2 or 3,
wherein the compound is other than N-phenyl-6-(4-phenylpiperazin-1-yl)pyrimidin-4-amine, N-phenyl-6-(4-piperazin-1-yl)pyrimidin-4-amine, and N-phenyl-6-piperidin-1-ylpyrimidin-4-amine.

4. A compound as defined in claim 1, wherein the compound has having the structural Formula (IV): 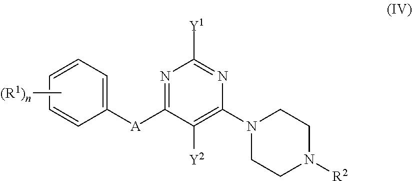
or a pharmaceutically acceptable salt thereof,
wherein:
A is —NH—, —NHC(O)—, —S—, —O—, —CH₂—, C(O)—, —S(O)—, or —S(O₂)—;
Y¹ and Y² are independently hydrogen, halogen, C₁₋₁₆alkyl, —O—C₁₋₁₆alkyl, —S—C₁₋₁₆alkyl, —S(O)—C₁₋₁₆alkyl, —S(O₂)—C₁₋₁₆alkyl, —C(O)—C₁₋₁₆alkyl, a cycloalkyl group, a heterocyclic group, a heteroaromatic group, or an aryl group, wherein the —C₁₋₁₆alkyl, —O—C₁₋₁₆alkyl, cycloalkyl group, S—C₁₋₁₆alkyl, —S(O)—C₁₋₁₆alkyl, —S(O)₂—C₁₋₁₆alkyl, —C(O)—C₁₋₁₆alkyl, heterocyclic group, heteroaromatic group, and aryl group are optionally substituted with one or more substituents independently selected from halogen, hydroxyl, oxo, lower alkyl, lower alkoxyl, and phenyl;
R² is —Y—R³;
Y is absent, —C(O)—, —SO—, —SO₂—, or —(CO)NH—;
R³ is hydrogen, —C₁₋₁₆alkyl, —O—C₁₋₁₆alkyl, —S—C₁₋₁₆alkyl, —S(O)—C₁₋₁₆alkyl, —S(O₂)—C₁₋₁₆alkyl, —C(O)—C₁₋₁₆alkyl, a cycloalkyl group, a heterocyclic group, a heteroaromatic group, or an aryl group, wherein the —C₁₋₁₆alkyl, —O—C₁₋₁₆alkyl, cycloalkyl group, S—C₁₋₁₆alkyl, —S(O)—C₁₋₁₆alkyl, —S(O₂)—C₁₋₁₆alkyl, —C(O)—C₁₋₁₆alkyl, heterocyclic group, heteroaromatic group, and aryl group are optionally substituted with one or more substituents independently selected from halogen, hydroxyl, oxo, lower alkyl, lower alkoxyl, halogenated lower alkyl, halogenated lower alkoxy, and phenyl;
R¹ is halogen, hydroxyl, —C₁₋₁₆alkyl, —O—C₁₋₁₆alkyl, S—C₁₋₁₆alkyl, —S(O)—C₁₋₁₆alkyl, —S(O₂)—C₁₋₁₆ alkyl, —C(O)—C₁₋₁₆alkyl or —N(R^{b})₂, wherein R^{b} is —H or —C₁₋₆alkyl, wherein each alkyl is optionally substituted with one or more halogens selected from F, Cl and Br; and
n is 0, 1, 2 or 3,
wherein the compound is other than N-phenyl-6-(4-phenylpiperazin-1-yl)pyrimidin-4-amine, N-phenyl-6-(4-piperazin-1-yl)pyrimidin-4-amine, and N-phenyl-6-piperidin-1-ylpyrimidin-4-amine.

5. A compound having the structural Formula (V): 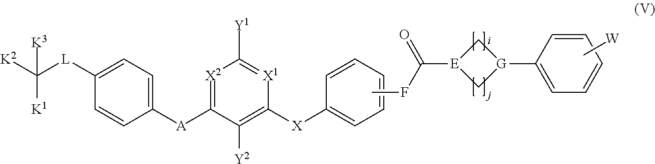
or a pharmaceutically acceptable salt thereof,
wherein:
X¹ and X² are each independently N or CH;
A is —NH—, —N(CH₃)—, —N(OH)—, —N(OCH₃)—, —NHC(O)—, —OC(O)NH—, —NHC(O)NH—, —NHS(O)₂—, —NHS(O)₂NH—, —S—, —O—, —CH₂—, —C(O)—, —S(O)—, or —S(O)₂—;
X is absent, —NH—, —N(CH₃)—, —N(OH)—, —N(OCH₃)—, —C(O)—, —SO₂—, —NHC(O)—, —NH(CO)NH—, —NHS(O)₂—, —NHS(O)₂NH—, —CH₂—, —SO—, a benzoyl moiety, or a phenyl moiety,
E is CH or N;
F is absent, —O—, —NH—, or —N(OH)—;
G is N or CH;
W is —CH₃, —CF₃, —OCF₃, OCH₂CF₃, or —CH₂CH₃;
Y¹ and Y² are each independently hydrogen, halogen, C₁₋₁₆alkyl, OH, —O—C₁₋₁₆alkyl, —S—C₁₋₁₆alkyl, —S(O)—C₁₋₁₆alkyl, —S(O₂)—C₁₋₁₆alkyl, —C(O)—C₁₋₁₆alkyl, a cycloalkyl group, a heterocyclic group, a heteroaromatic group, or an aryl group, wherein the —C₁₋₁₆alkyl, —O—C₁₋₁₆alkyl, cycloalkyl group, S—C₁₋₁₆alkyl, —S(O)—C₁₋₁₆alkyl, —S(O₂)—C₁₋₁₆alkyl, —C(O)—C₁₋₁₆alkyl, heterocyclic group, heteroaromatic group, and aryl group are optionally substituted with one or more substituents independently selected from halogen, hydroxyl, oxo, lower alkyl, lower alkoxyl, and phenyl;
R³ is hydrogen, —C₁₋₁₆alkyl, —O—C₁₋₁₆alkyl, —S—C₁₋₁₆alkyl, —S(O)—C₁₋₁₆alkyl, —S(O)₂—C₁₋₁₆alkyl, —C(O)—C₁₋₁₆alkyl, a cycloalkyl group, a heterocyclic group, a heteroaromatic group, or an aryl group, wherein the —C₁₋₁₆alkyl, —O—C₁₋₁₆alkyl, cycloalkyl group, S—C₁₋₁₆alkyl, —S(O)—C₁₋₁₆alkyl, —S(O)₂—C₁₋₁₆alkyl, —C(O)—C₁₋₁₆alkyl, heterocyclic group, heteroaromatic group, and aryl group are optionally substituted with one or more substituents independently selected from halogen, hydroxyl, oxo, lower alkyl, lower alkoxyl, halogenated lower alkyl, halogenated lower alkoxy, and phenyl;
i is 1, 2, 3 or 4;
j is 1, 2, 3 or 4;
L is —NH—, —NHC(O)—, —S—, —O—, —CH₂—, C(O)—, —S(O)—, or —S(O₂)—;
K¹, K² and K³ are each independently —F, —C, —Br, —I, —CH₃, —OH, CF₃, —NHS(O)₂CF₃, or —NHS(O)₂CH₃.

6. A compound having the compounds of structural Formula (VI): 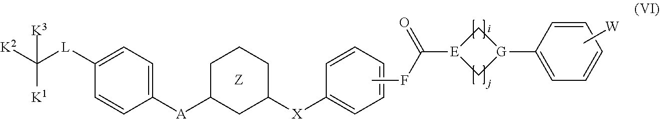
or a pharmaceutically acceptable salt thereof,
wherein:
Z is selected from 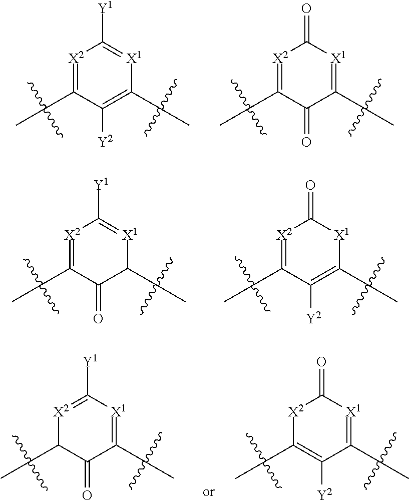
X¹ and X² are each independently N or CH;
A is —NH—, —N(CH₃)—, —N(OH)—, —N(OCH₃)—, —NHC(O)—, —OC(O)NH—, —NHC(O)NH—, —NHS(O)₂—, —NHS(O)₂NH—, —S—, —O—, or —CH₂—, C(O)—, —S(O)—, or —S(O)₂—;
X is absent, —NH—, —N(CH₃)—, —N(OH)—, —N(OCH₃)—, —C(O)—, —SO₂—, —NHC(O)—, —NH(CO)NH—, —NHS(O)₂—, —NHS(O)₂NH—, —CH₂—, —SO—, a benzoyl moiety, or a phenyl moiety,
E is CH or N;
F is absent, —O—, —NH—, or —N(OH)—;
G is N or CH;
W is —CH₃, —CF₃, —OCF₃, OCH₂CF₃, or —CH₂CH₃;
Y¹ and Y² are each independently hydrogen, halogen, C₁₋₁₆alkyl, OH, —O—C₁₋₁₆alkyl, —S—C₁₋₁₆alkyl, —S(O)—C₁₋₁₆alkyl, —S(O₂)—C₁₋₁₆alkyl, —C(O)—C₁₋₁₆alkyl, a cycloalkyl group, a heterocyclic group, a heteroaromatic group, or an aryl group, wherein the —C₁₋₁₆alkyl, —O—C₁₋₁₆alkyl, cycloalkyl group, S—C₁₋₁₆alkyl, —S(O)—C₁₋₁₆alkyl, —S(O₂)—C₁₋₁₆alkyl, —C(O)—C₁₋₁₆alkyl, heterocyclic group, heteroaromatic group, and aryl group are optionally substituted with one or more substituents independently selected from halogen, hydroxyl, oxo, lower alkyl, lower alkoxyl, and phenyl;
R³ is hydrogen, —C₁₋₁₆alkyl, —O—C₁₋₁₆alkyl, —S—C₁₋₁₆alkyl, —S(O)—C₁₋₁₆alkyl, —S(O)₂—C₁₋₁₆alkyl, —C(O)—C₁₋₁₆alkyl, a cycloalkyl group, a heterocyclic group, a heteroaromatic group, or an aryl group, wherein the —C₁₋₁₆alkyl, —O—C₁₋₁₆alkyl, cycloalkyl group, —S—C₁₋₁₆alkyl, —S(O)—C₁₋₁₆alkyl, —S(O)₂—C₁₋₁₆alkyl, —C(O)—C₁₋₁₆alkyl, heterocyclic group, heteroaromatic group, and aryl group are optionally substituted with one or more substituents independently selected from halogen, hydroxyl, oxo, lower alkyl, lower alkoxyl, halogenated lower alkyl, halogenated lower alkoxy, and phenyl;
i is 1, 2, 3 or 4;
j is 1, 2, 3 or 4;
L is —NH—, —NHC(O)—, —S—, —O—, —CH₂—, —C(O)—, —S(O)—, or —S(O₂)—, or —NHS(O)₂—;
K¹, K² and K³ are each independently —F, —C, —Br, —I, —CH₃, —OH, CF₃, —NHS(O)₂CF₃, or —NHS(O)₂CH₃.

7. A compound as defined in claim 1, wherein B is NR².

8. A compound as defined in claim 7, wherein R² is YR³, wherein Y is absent, —C(O)—, or —SO₂—, and R³ is an aryl group optionally substituted with one or more substituents independently selected from halogen, hydroxyl, lower alkyl, lower alkoxyl, halogenated lower alkyl, and phenyl.

9. A compound as defined in claim 1, wherein R¹ is CF₃ and n is 1.

10. A compound as defined in claim 1, wherein A is NH.

11. A compound as defined in claim 1, wherein X¹ and X² are each N.

12. A compound as defined in claim 1, wherein Y¹ and Y² are each H.

13. A pharmaceutical composition comprising a compound as defined in claim 1, and a pharmaceutically acceptable carrier or diluent.

14. A method of treating or preventing cancer in a subject in need thereof, the method comprising the step of administering a therapeutically effective amount of a compound as defined in claim 1, or a pharmaceutically acceptable salt or solvate thereof, to the subject.

15. A method of overcoming cancer resistance in a subject in need thereof, the method comprising the step of administering a therapeutically effective amount of a compound as defined in claim 1, or a pharmaceutically acceptable salt or solvate thereof, to the subject.

16.-19. (canceled)

20. A method of treating or preventing cancer in a subject in need thereof, the method comprising the step of administering a therapeutically effective amount of a compound as defined in claim 1, or a pharmaceutically acceptable salt or solvate thereof, to the subject, wherein the compound is administered in combination with a known chemotherapeutic agent.

21. A method of overcoming cancer resistance in a subject in need thereof, the method comprising the step of administering a therapeutically effective amount of a compound as defined in claim 1, or a pharmaceutically acceptable salt or solvate thereof, to the subject, wherein the compound is administered in combination with a known chemotherapeutic agent.

22. The method of claim 20, wherein the known chemotherapeutic agent is selected from the group consisting of imatinib, nilotinib, dasatinib, bosutinib, ponatinib, bafetinib, rebastinib, tozasertib and danusertib.

23.-25. (canceled)

26. The method of claim 21, wherein the known chemotherapeutic agent is selected from the group consisting of imatinib, nilotinib, dasatinib, bosutinib, ponatinib, bafetinib, rebastinib, tozasertib and danusertib.

27. A compound as defined in claim 1, wherein the compound is selected from the group consisting of: 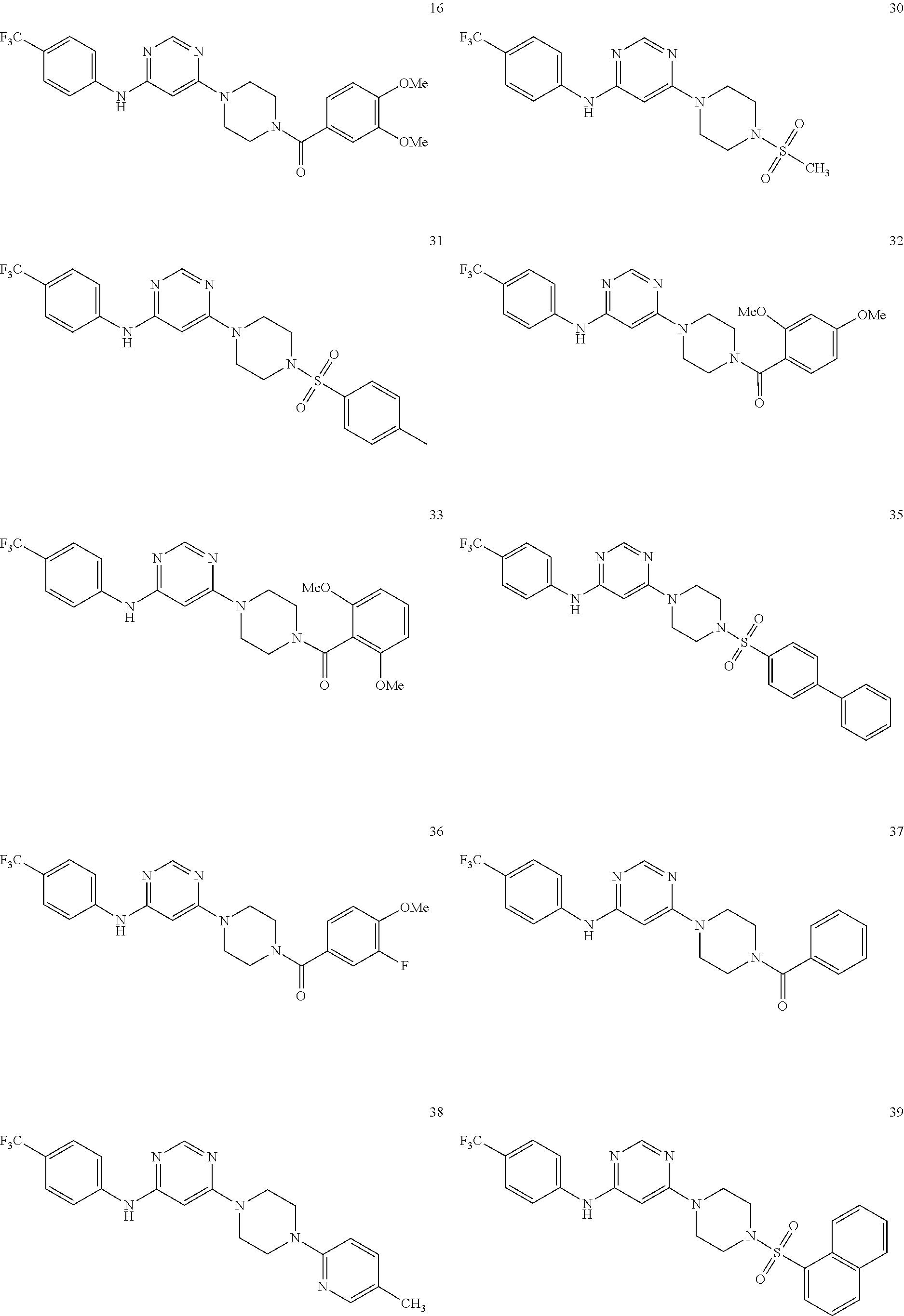 [chemical structure]
